(19) United States
(12) Patent Application Publication
(10) Pub. No.: US 2023/0098031 A1
(43) Pub. Date: Mar. 30, 2023

(54) METHODS FOR CELL IMAGING

(71) Applicant: The General Hospital Corporation, Boston, MA (US)

(72) Inventors: Ralph Weissleder, Peabody, MA (US); Jonathan C.T. Carlson, Boston, MA (US); Jina Ko, Boston, MA (US)

(21) Appl. No.: 17/783,214

(22) PCT Filed: Dec. 10, 2020
(86) PCT No.: PCT/US2020/064232

§ 371 Date: Jun. 7, 2022

Related U.S. Application Data
(60) Provisional application No. 62/967,814, filed on Jan. 30, 2020.
(60) Provisional application No. 62/967,586, filed on Jan. 29, 2020.
(60) Provisional application No. 62/946,863, filed on Dec. 11, 2019.

(51) Int. Cl. G01N 33/58; C07C 271/22; C07D 403/12; C07D 207/404; G01N 33/574

(52) U.S. Cl. CPC G01N 33/582; C07C 271/22; C07D 403/12; C07D 207/404; G01N 33/57492

(57) ABSTRACT

The present application provides a compound of Formula (A), or a pharmaceutically acceptable salt thereof, wherein R¹, L¹, n, L², m, L³, p, Y², and Y³ are as described herein. Methods of using the compound of Formula (A) to prepare an antibody conjugate, and methods of using these conjugates for cellular imaging are also described.

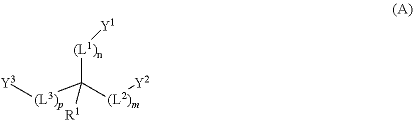

### CLAIM OF PRIORITY

This application claims priority to U.S. Provisional Patent Application Ser. No. 62/967,814, filed on Jan. 30, 2020, U.S. Provisional Patent Application Ser. No. 62/967,586, filed on Jan. 29, 2020, and U.S. Provisional Patent Application Ser. No. 62/946,863, filed Dec. 11, 2019, the entire contents of which are hereby incorporated by reference.

### TECHNICAL FIELD

This invention relates to tridentate ligands, and methods of using these ligands for cellular fluorescence imaging, including multiplexed cellular fluorescence imaging.

### BACKGROUND

Processing cellular samples, e.g., for immunostaining and image cytometry, often can be quite challenging. One drawback is that the cellular samples are often scant (often <1,000 cells per pass from a fine needle aspirate), limiting the number of special stains that can be done, and also delicate, lacking the structural scaffold of intact tissue architecture. Even when processed with fluorescent antibodies, the number of different stains for cellular samples is typically limited to 4-6 and is often insufficient for in depth cancer cell profiling for diagnosis or treatment assessment. This limitation also extends to immune profiling, where significantly more than 4-6 markers need to be interrogated so that analysis reflects the representative immunocyte populations in the tumor microenvironment.

### SUMMARY

Most fluorescent cycling methods were originally developed for paraffin embedded tissue sections that can withstand harsh destaining and quenching conditions. These harsh conditions often require oxidants for bleaching and, therefore, are not compatible with cellular samples such as those in fine needle aspirates (FNA). Furthermore, it is not uncommon for antibody-DNA cycling technologies to take hours or even days of sample processing, as, for example, in the ABCD and SCANT methods. Described herein are ultrafast methods of single cell cycling through the use of clickable linkers for fluorophores and quenchers. Although such methods are applicable to various tissue samples, including paraffin-embedded tissue sections, these methods supersede the requirements for bleaching or cleavage in the conventional methods. In one embodiment of the present disclosure, the tetrazine (Tz)/trans-cyclooctene (TCO) click chemistry approach allowed for site-specific delivery of fluorescence quenchers followed by efficient quenching across the color spectrum and a remarkable acceleration in the chemical reaction kinetics. That is, unexpectedly, in the methods within the present claims, the click reaction is up to 10⁴ times faster than predicted by the kinetics of the conventional click chemistry. This advantageously allows ultra-fast (<1 sec) quenching of fluorescence in clinical specimens and allows multichannel imaging of 20-30 markers within an hour.

In one general aspect, the present disclosure provides a compound of Formula (A):

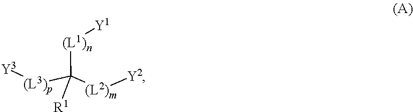

or a pharmaceutically acceptable salt thereof, wherein R¹, L¹, n, L², m, L³, p, Y¹, Y², and Y³ are as described herein.

In another general aspect, the present disclosure provides a compound of Formula (I):

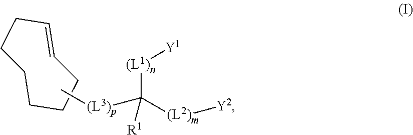

or a pharmaceutically acceptable salt thereof, wherein R¹, L¹, n, L², m, L³, p, Y¹, and Y² are as described herein.

In yet another general aspect, the present disclosure provides a protein conjugate of Formula (B):

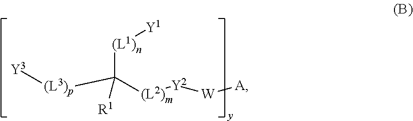

or a pharmaceutically acceptable salt thereof, wherein R¹, L¹, n, L², m, L³, p, Y¹, Y², Y³, W, A, and y are as described herein.

In yet another general aspect, the present disclosure provides A protein conjugate of Formula (II):

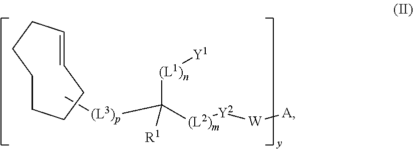

or a pharmaceutically acceptable salt thereof, wherein R¹, L¹, n, L², m, L³, p Y¹, Y², W, A, and y are as described herein.

In yet another general aspect, the present disclosure provides a composition comprising the protein conjugate as described herein, or a pharmaceutically acceptable salt thereof, and an inert carrier.

In yet another general aspect, the present disclosure provides a method of examining a cell or a component of a cell, the method comprising:
(i) contacting the cell with a conjugate as described herein comprising the residue of the fluorophore, or a pharmaceutically acceptable salt thereof, or a composition comprising same;
(ii) imaging the cell with an imaging technique; and
(iii) after (ii), contacting the cell with a compound of Formula (C):

Y⁴-(L⁴)ₐ-Q (C),

or a pharmaceutically acceptable salt thereof, wherein Y⁴, L⁴, a, and Q are as described herein.

In yet another general aspect, the present disclosure provides a method of examining a cell or a component of a cell, the method comprising:
(iv) contacting the cell with a conjugate as described herein comprising the residue of the fluorophore, or a pharmaceutically acceptable salt thereof, or a composition comprising same;
(v) imaging the cell with an imaging technique; and
(vi) after (ii), contacting the cell with a compound of Formula (III):

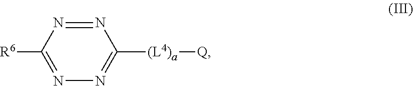

or a pharmaceutically acceptable salt thereof, wherein R⁶, L⁴, a, and Q are as described herein.

In yet another general aspect, the present disclosure provides a method of profiling a cell, the method comprising (i) obtaining the cell from a subject, and (ii) examining the cell according to the method of the present disclosure.

In yet another general aspect, the present disclosure provides a method of examining a cell using a cytometry technique, the method comprising (i) obtaining the cell from a subject, and (ii) examining the cell according to the method of the present disclosure.

In yet another general aspect, the present disclosure provides a method of diagnosing a disease or condition of a subject by examining pathology of a cell obtained from the subject, the method comprising (i) obtaining the cell from a subject, and (ii) examining the cell according to the method of the present disclosure.

In yet another general aspect, the present disclosure provides a method of monitoring progression of disease or condition of a subject by examining pathology of a cell obtained from the subject, the method comprising (i) obtaining the cell from the subject, and (ii) examining the cell according to the method of the present disclosure.

In yet another general aspect, the present disclosure provides a method of detecting a disease biomarker in a cell, the method comprising (i) obtaining the cell from a subject, and (ii) examining the cell according to the method of the present disclosure.

In yet another general aspect, the present disclosure provides a method of preparing an activated ester of a compound comprising a carboxylic acid group, the method comprising i) reacting the compound comprising a carboxylic acid group with an excess amount of an activating reagent to obtain a reaction mixture comprising the activated ester; and ii) contacting the reaction mixture with a compound of Formula (D):

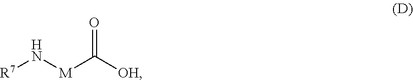

or a pharmaceutically acceptable salt thereof, wherein R⁷ and M are as described herein, wherein the contacting of the reaction mixture obtained in step i) with the compound of Formula (I), or a pharmaceutically acceptable salt thereof, deactivates the excess of the activating reagent in the reaction mixture.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1A is a schematic illustration of the synthesis route for preparation of TCO-linked fluorophores (FAST probes) built on a lysine scaffold with a PEG4 linker for efficient antibody conjugation: i). TSTU, DIPEA; ii). H2N-PEG4-CO₂H; iii). DCM/TFA (20%); iv). rTCO(axial)-PNP, DIPEA; v). piperidine (7.5%). The core linker can be functionalized with any amine-reactive fluorophore of choice. In addition, excess TSTU can be used for rapid activation of the PEG4-COOH and neutralized immediately thereafter with ENBA, circumventing a range of purification and antibody-conjugation obstacles.

FIG. 1B is a schematic illustration of a synthetic route for coupling of BHQ3-amine with HTz-PEG₅-NHS to yield BHQ3-Tz in one step.

FIG. 1C is a structural schematic of the BHQ3-fluorophore quenching interaction after TCO-Tz click (AF647 fluorescent dye is depicted in the schematic).

FIG. 2A contains quantitative data of labeling and quenching efficiency. (i) AF647-rTCO (FAST-AF647) labeling of an anti-CD4 antibody produced bright conjugates with excellent efficiency and staining brightness proportional to DOL, matching or exceeding the brightness of the commercial antibody (FI-MAb). (ii) Representative images of mouse splenocytes stained with a FAST-AF647 anti-CD4 antibody and the FI-MAb reference.

FIG. 2B contains data showing quenching efficiency assessed by fluorimeter and by microscopy. Quenching efficiency assessed by fluorimeter and by microscopy. (i) The fluorescence emission spectrum of FAST-AF647 anti-CD4 demonstrates >99% reduction in signal after treatment with BHQ3-Tz in PBS. (ii) Measurement of residual MFI in A431 cells after quenching also showed efficient reduction in the brightness of FAST-labeled anti-EGFR antibody (cetuximab) staining for four different dyes (AF488, AF555, AF594, and AF647). (iii) Representative images of EGFR staining and quenching in A431 cells. Cells were stained with AF488-rTCO or AF594-rTCO cetuximab (5 μg/mL) and imaged before and after (at right) quenching with 20 μM BHQ3-Tz for 5 minutes.

FIG. 2C contains line plots showing rTCO release from the conjugates. To quantify any impact of rTCO release, BHQ3-Tz quenching of FAST-AF647 labeled cetuximab was monitored longitudinally as a function of pH. While a slow rebound in brightness is evident at mildly acidic pH, consistent with partial release of BHQ3-Tz/rTCO from the antibody, quenching is exceptionally stable at pH 9.

FIG. 3A contains an image showing that A431 cells stained with FAST-AF647 cetuximab and DAPI for nuclear reference were treated with 20 μM BHQ3-Tz in PBS-bicarb (pH 9) for progressively shorter amounts of time. Quenching remained near-quantitative after incubation for as little as 10 seconds.

FIG. 3B contains plots showing experimental design for fluorescence kinetics: FAST-AF488 was selected for initial studies to minimize spectral interference between ex/em wavelengths and the BHQ3 absorbance. At just 2 μM BHQ3-Tz in PBS, quenching of labeled Ab fluorescence was exceptionally rapid (shaded interval, 2 seconds).

FIG. 3C contains line plots showing that systematic kinetics for AF647, AF594 and AF488 probes in PBS-bicarb (pH 9) as a function of BHQ3-Tz concentration revealed remarkable dye-specific accelerations in click rates relative to the expected rate for the Tz-TCO pair.

FIG. 3D contains line plots showing that kinetic studies of FAST-labeled antibodies in PBS-bicarb (pH 9) demonstrate that the cumulative acceleration is even greater for the multivalent antibodies and dependent on dye, DOL, and BHQ3-Tz concentration.

FIG. 4 contains images showing the use of three FAST probes (AF488, AF594, AF647) to stain multiple targets in single cells. Anti-pS6 was stained with AF488-rTCO, anti-EGFR with AF594-rTCO, and anti-S6 with AF647-rTCO to validate multi-target imaging ability of the FAST probes. After antibody staining (1-5 g/mL) and washing, the cells were incubated with BHQ3-Tz (10 μM in PBS-Bicarb, pH 9, one minute) for quenching. Intensity profiles before and after quenching are included to show the decrease of the fluorescent signals from all three targets back to the background level with highly efficient quenching.

FIG. 5A contains images obtained by cyclic imaging of immune markers in a mouse tumor FNA sample. Twelve markers were imaged using three FAST-probe fluorophores (AF647: red, AF594: magenta, AF488: green in the images) in four imaging cycles. All images show the same set of cells within a zoomed-in area of a single field of view in order to appreciate the patterns of fluorescence signal of immune markers expressed in individual cells. DAPI was used to stain the nuclei of all cells imaged in each cycle for cycle-to-cycle alignment (scale bar: 20 μm).

FIG. 5B contains summary of data from a total of 1846 cells analyzed, the frequencies of different immune cell types (CD8+ T cells, CD4+ T cells, macrophage, dendritic cells, neutrophils) and key subsets (PD-1, CD163/CD206) in CD45+ immune cells were quantified. Each immune cell type was identified using the selected combinations of markers as indicated.

FIG. 6A contains a schematic showing that TCO labeled fluorescent antibodies are efficiently quenched on reaction with a BHQ-tetrazine. Commercial secondary antibodies were treated with TCO-PEG₄-NHS (Click Chemistry Tools) to randomly attach 3-6 TCOs per antibody and purified by gel filtration spin column. Aliquots of the TCO-labeled antibodies were treated with Tz-BHQ₁₀ in excess and then purified again by gel filtration spin column. Degree of labeling was quantified by ratiometric absorbance measurements and the known wavelength-specific extinction coefficients of the antibody (IgG, 280 nm, 210000 M⁻¹cm⁻¹), the respective fluorophores, and the BHQ₁₀.

FIG. 6B is a line plot showing fluorescence emission spectra of the antibodies before and after BHQ3-Tz labeling.

FIG. 6C is a bar graph showing quantitative quenching efficiency by dye.

FIG. 7A shows in situ NHS formation and TSTU quenching with ENBA. ENBA (4-(ethylamino)butanoic acid) does not react with NHS esters at room temperature. A test reaction with N-α-Boc-N-ε-TFA-Lysine NHS (Chem-Impex) demonstrates no reaction of the NHS ester after one hour in DMSO solution with 10 mM ENBA.

FIG. 7B contains a scheme and a line plot showing that ENBA reacts rapidly with TSTU to form ENBA-NHS, which undergoes rapid intramolecular cyclization to yield N-ethyl-2-pyrrolidone (readily detected by LCMS, m/z 114.1). Serial analyses of the reaction mixture revealed complete conversion to N-ethyl-2-pyrrolidone in <2 minutes, neutralizing the TSTU without generating any new active esters that could go on to react with the antibody during labeling.

FIG. 8A is a bar graph showing quenching efficiencies using different BHQ types and a variety of fluorophores. TCO labeled AF488, AF594, and AF647 secondary antibodies were quenched with either BHQ10-Tz or BHQ3-Tz for quenching efficiency comparison. BHQ2-Tz conjugates were also prepared, but proved insufficiently soluble for use in this format.

FIG. 8B is a bar graph showing a broad spectrum of TCO labeled fluorescent antibodies (OG488, AF488, AF532, AF594, AF647, IR750) were quenched with either BHQ3-TZ or IRdyeQC1-Tz.

FIG. 9 contains stained and quenched fluorescent images using four FAST probes (AF488, AF555, AF594, AF647). A431 cells were stained with different rTCO-dyes for 20 minutes and imaged before and after quenching with BHQ3-Tz (10 μM) for 3 minutes. The same brightness/contrast settings were applied to before/after quenching for comparison. Quantitative intensity analyses of these images are presented in FIG. 2B.

FIG. 10A is an image showing that conventional fluorescent antibodies are not quenched by BHQ3-Tz. A431 cells were stained in parallel with cetuximab-FAST-AF647 and a conventionally labeled cetuximab-Alexa Fluor 647 conjugate (at right). Imaging revealed well-matched brightness for the paired antibodies. After treating both wells with 20 μM BHQ3-Tz (5 min) in PBS, repeat imaging revealed complete quenching of the AF647 signal in the FAST-stained cells and no change in the brightness of the conventionally labeled antibody/cells.

FIG. 10B is a line plot showing conventional secondary antibodies labeled with each of the four fluorophores used for FAST-probes were diluted into disposable fluorescence cuvettes that had been blocked with 40 μg/mL cetuximab to prevent nonspecific adsorption. After recording the baseline fluorescence intensity, 1-2 μM BHQ3-Tz was added to the cuvette. No significant change in brightness was observed for any of the four dyes. For comparison, the Cetux-Fast488 trace from FIG. 3B is overlaid on the same timescale and the BHQ3-Tz addition synchronized.

FIG. 11A contains line plots showing that FAST-antibody quenching kinetics are consistent across multiple antibodies. Quenching profiles for FAST-AF647 labeled CD45 antibodies (2 nM) and BHQ3-Tz match those observed for cetuximab (FIG. 3B,3D), with rates that depend on DOL and on the concentration of BHQ3-Tz. Relative accelerations are calculated from the observed pseudo-first order rate constants (k_{fast}) and the expected 7173 M⁻¹s⁻¹ for the rTCO-benzylaminoTzH pair.

FIG. 11B is a table showing nonlinear fits and confidence intervals for the data presented in FIG. 11A.

FIG. 11C contains a line plot showing FAST-antibody kinetic acceleration in biological media. Quenching profiles for BHQ3-Tz and FAST-AF647-labeled Cetuximab (7 nM, 1 μg/mL) in PBS with and without added BSA. The buffer solution was added to the cuvette and allowed to stir for 30-60 sec, followed by the antibody; fluorescence intensity was monitored continuously at baseline and after the addition of BHQ3-Tz (100 nM). Kinetics in the presence of excess BSA display a subtle multi-phase decay that is not well-fitted to a single/double exponential, but remain markedly accelerated relative to the predicted rate (dashed line).

FIG. 11D contains a line plot showing FAST-antibody kinetic acceleration in biological media. Quenching profiles in cell culture media were collected as in FIG. 11C; FluoroBrite DMEM (FB-DMEM, Gibco) was used to minimize background fluorescence. Kinetics in serum free FB-DMEM are minimally altered relative to PBS; the kinetic profile with complete media (FBDMEM with 10% fetal bovine serum) is similar to that observed for 1% BSA and retains the marked acceleration.

FIG. 12 contains images showing CD45+ cell segmentation for immune cell population analysis. FNA samples imaged in cycle one were stained for both CD45•FAST-AF647 and DAPI in the marker set to guide selection of fields of view with suitable immune cell density for profiling and to build a map of CD45 positive cells. These two images were integrated to build the CellProfiler segmentation map of the CD45+ immune cells in each field of view, as shown at bottom right. The dotted box in the CD45•FAST-AF647 image (upper left) outlines the specific area selected and expanded (FIG. 5A) to highlight immune marker expression.

FIG. 13 is a table containing a list of antibodies for FAST staining of immunocyte populations. Antibodies used for analyses of immune cell populations from MC38 tumor FNA samples (FIG. 5), including degree of labeling.

FIG. 14A contains line plots showing kinetic data for AF488-rTCO-P₄+BHQ3-Tz.

FIG. 14B contains line plots showing kinetic data for AF647-rTCO-P₄+BHQ3-Tz.

FIG. 15 is a synthetic scheme illustrating chemical synthesis of a FAST probe scaffold (4).

FIG. 16A contains chemical structures of FAST-AF488 (4a) and FAST-AF555 (4b).

FIG. 16B contains chemical structures of FAST-AF594 (4c) and FAST-AF647 (4d).

FIG. 17 is an overview diagram with clinical needs and turnaround times. Scant cells can be obtained by fine needle aspiration (FNA), brushings, touch preps or blood/fluid samples. Essential to the integrated and automated processing of such cells are cycling methods, instrumentation and computational approaches. Indeed the analysis relies heavily on deep learning and AI approaches to extract information from dozens of channels and convert them into a medical diagnosis. For point-of-care settings, all of the above occur within reasonable time frames and at low cost. DL, deep learning; AI, artificial intelligence.

FIG. 18 contains schemes and images showing cyclic labeling technologies for multiplexed assessment of cancer and host cell markers; different cycling techniques and an example of immune cell profiling in FNA sample using cell based cycling.

FIG. 19 is a table containing overview of some experimental (top) and commercial systems (bottom),

FIG. 20A contains a structural scheme of a miniscope. A finger-sized, single-channel fluorescent microscope is structured like a conventional fluorescent microscope but uses an LED as an excitation source and a gradient refractive index (GRIN) lens as an objective.

FIG. 20B is an image of Mycobacterium tuberculosis stained with auramine-O. The image shows 300×300 pixel regions of the CMOS camera.

FIG. 20C is an image of Cytometry Portable Analyzer (CytoPAN). The system is integrates five light sources and a quad-band filter. No mechanical parts are necessary for multiple channel imaging.

FIG. 20D is an image of the analysis of an FNA specimen from a breast cancer patient. Cancer cells were identified through the staining of QUAD markers: EGFR, EpCAM, HER2, MUC1 or EGFR, EpCAM, CK, MUC1. Immune cells through CD45 staining. Images were taken at 5× magnification.

FIG. 20E shows that CytoPAN software automatically profiles individual cells in multi-color channels and generates a summary report to guide cancer diagnosis.

FIG. 21 is a table showing comparison of some cellular cycling techniques. The table provides an overview of three recently developed technologies: ABCD, SCANT and the methods and compounds of the present application (FAST). Collectively, the technologies allow imaging of 20-40 targets in each individual cells and this can be used for cellular mapping (e.g. immune cell profiling), cellular pathway analysis or heterogeneity studies.

FIG. 22A contains chemical structures of FAST-AF488-NHS and FAST-AF555-NHS.

FIG. 22B contains chemical structures of FAST-AF594-NHS and FAST-AF647-NHS.

FIG. 23 is a synthetic scheme showing chemical synthesis of key intermediate (6) for the preparation of FAST 5-OH TCO probes.

FIG. 24 contains chemical structures of FAST 5-OH TCO-AF488 (compound 6a) and FAST 5-OH TCO-Oregon Green (compound 6b).

FIG. 25 contains chemical structures of FAST 5-OH TCO-AF532 (compound 6c) and FAST 5-OH TCO-AF594 (compound 6d).

FIG. 26 contains chemical structures of FAST 5-OH TCO-AFDye 647 (compound 6e) and FAST 5-OH TCO-IRDye750 (compound 6f).

FIG. 27 contains a scheme showing chemical synthesis of double 5-OH TCO intermediate reagent (10) for the preparation of double 5-OH TCO probes.

FIG. 28 contains chemical structures of double 5-OH TCO-NHS reagents.

FIG. 29 contains chemical structures of double 5-OH TCO probes (compounds 10a and 10b).

FIG. 30 contains a scheme showing chemical synthesis of double dTCO intermediate reagent (14) for the preparation of dTCO probes.

FIG. 31 contains chemical structures of double dTCO probes (compounds 14a and 14b).

FIG. 32 contains a scheme showing chemical synthesis of cyclopropane probe (17) via the key intermediate (16).

FIG. 33 contains a scheme showing chemical synthesis of a key intermediate (33) for the preparation of tetrazine (TZ) probe.

FIG. 34A contains a scheme showing chemical synthesis of ternary TCO reagent (23) from triamino(trideoxy)inositol.

FIG. 34B contains a scheme showing chemical synthesis of ternary TCO reagent (24) from tris(2-aminoethyl)amine.

FIG. 35 contains a scheme showing chemical synthesis of a double TCO probe intermediate (26) via ternary TCO reagent (23).

FIG. 36 contains a scheme showing chemical synthesis of a double TCO probe intermediate (28) via ternary TCO reagent (24).

FIG. 37A contains a bar graph showing quenching efficiency of compounds 6a and 10b using BHQ3 and BHQ10 as a quencher.

FIG. 37B contains image of A431 cells stained with compounds 10b, 6a, and 6e conjugated to antibody cetuximab.

FIG. 38A contains image of A431 cells stained with compound 14b conjugated to antibody cetuximab.

FIG. 38B contains image of cells stained with compound 14b conjugated to anti-CD3 antibody.

FIG. 39 contains a line plot showing results of quenching kinetics experiments using compound 17 and BHQ3-Tz.

FIG. 40 contains images showing staining and quenching of live and fixed A431 cells using compound 4d.

FIG. 41 contains images showing FAST-FNA cyclic imaging of immune cells in the tumor environment. An example field of view of MC38 tumor FNA in the first three cycles of FAST imaging. Images are zoomed in to show different staining patterns of the immune cell markers. In each cycle three markers were stained with FAST antibodies equipped with AF488, AF555 or AF647. After imaging, the fluorescent signals were quenched before the new antibody staining of the next cycle. Nuclear staining by DAPI was imaged each cycle to facilitate image registration. (Scale bar 50 μm).

FIG. 42A shows validation of FAST-FNA analysis on mouse tumor models and serial FNA analysis. An example of a typical FNA sample analysis. For validation of the FAST-FNA analysis, FNA samples were collected from mouse MC38 tumors implanted on B6 mice

FIG. 42B shows validation of FAST-FNA analysis on mouse tumor models and serial FNA analysis. A total of 16 markers were analyzed through 6 image cycles to fractionate tumor cells from leukocytes and identify monocytes, macrophages, dendritic cells, neutrophils, natural killer cells, B cells, CD4+ T cells, regulatory T cells, CD8+ T cells and their subtypes. Cell counts of each immune cell type and its percentage in total cells are shown in the table and donut chart.

FIG. 42C shows validation of FAST-FNA analysis on mouse tumor models and serial FNA analysis. Correlation of the immune cell frequency results obtained by FAST-FNA assay with the results by flow cytometry of the same MC38 tumor homogenate (n=6; R²=0.97; slope 1.03 with 95% confidence interval 0.97-1.10 in the shaded area of the plot).

FIG. 42D shows validation of FAST-FNA analysis on mouse tumor models and serial FNA analysis. To test biological variation, FNA samples were collected from a MC38 tumor consecutively and their immune composition was compared. Immune cell type analyses of 5 consecutive FNA samples show variations within the 95% confidence interval of the mean (shaded areas).

FIG. 42E shows validation of FAST-FNA analysis on mouse tumor models and serial FNA analysis. Immune cell type analysis by FAST-FNA assay was repeated on the same cell stock of mouse spleen homogenate. Frequencies of various immune cell populations analyzed by 4 repeated trials also exhibits its variations within the 95% confidence interval of the mean (shaded areas).

FIG. 43A contains a bar graph showing FAST-FNA analysis of HNSCC human patient FNA samples. Analysis of HNSCC patient samples (n=9) by FAST analysis. Note the varying immune cell compositions.

FIG. 43B shows validation of FAST-FNA analysis on tumor samples biopsied after surgical excision. Correlation of the immune cell frequency results obtained by FAST-FNA assay with the results by flow cytometry of the homogenized tumor tissue (R²=0.86).

FIG. 43C shows longitudinal FAST-FNA analysis of HNSCC in a patient undergoing immunotherapy; serial biopsies collected every 2 or 3 weeks during treatment enable quantification of intratumoral immune population dynamics.

FIG. 44 contains a Table showing antibodies for FAST imaging of mouse immune cell populations.

FIG. 45 contains a Table showing antibodies for FAST imaging of human HNSCC specimens.

### DETAILED DESCRIPTION

Molecular analyses of cancer cells are essential in establishing diagnosis and guiding available treatments. [See Ref. 1] In an ideal world, one would like to harvest cancers frequently and in the least invasive manner so that molecular information can be obtained periodically through treatment and cancer evolution. [See Ref. 2] “Liquid biopsies”, i.e. the interrogation of circulating tumor cells [See Ref. 3], extracellular vesicles [See Ref 4], or cell-free DNA in the peripheral blood, provide one such option, but detection of actionable events is rare and overall sensitivities can be low. [See Ref 5] More importantly, circulating tumor diagnostics cannot currently be traced back to their anatomical origin, whether primary tumor or metastatic site. This limits the ability to correlate molecular events with radiographic/imaging measures of cancer behavior, invasiveness, and progression.

An alternative method is fine needle aspiration (FNA) that yields cells rather than tissue from a tumor, are inherently of known localization, and which can be processed expeditiously, i.e. do not require embedding or sectioning. FNA are obtained with small gauge needles (20-25 G) and are generally well tolerated. [See Ref. 6] As such, image guided FNA are ideally suited for repeat sampling and have a very low risk of procedural complications. However, as mentioned previously, the challenge in processing these cellular samples is that they can be scant (often <1,000 cells per pass), limiting the number of special stains that can be done, and also delicate, lacking the structural scaffold of intact tissue architecture. Even when processed with fluorescent antibodies, the number of different stains is practically limited to 4-6 and often not sufficient for in depth cancer cell profiling for diagnosis or treatment assessment. This limitation also extends to immune profiling, where significantly more than 4-6 markers need to be interrogated so that analysis reflects the representative immunocyte populations in the tumor microenvironment. [See Ref. 7] In contrast, single cell cycling methods of the present disclosure allow repeat staining, destaining, and re-staining of harvested cellular samples for better therapy assessment in both cancer cells and host immune cells.

Most fluorescent cycling methods [See Ref. 8] were originally developed for paraffin embedded tissue sections that can withstand harsh destaining/quenching conditions. Unfortunately, these harsh conditions typically require oxidants for bleaching at strongly alkaline pH (e.g., 4.5% H₂O₂, 24 mM NaOH, pH>12) and are not well suited for cellular FNA samples. Furthermore, it is not uncommon for other antibody-DNA cycling technologies to require a significant investment in nucleic acid tags/technologies and take hours-days of sample processing, including ABCD [See Ref 9] and SCANT [See Ref 10] Similar technical hurdles accompany other conventional methods for antibody-DNA based imaging, including intricate chemical steps for DNA barcode activation and antibody-DNA bioconjugation, and/or complex fluidics required for cycling multiple sequential staining solutions. [See Ref. 11]

As described more fully below, the present disclosure provides fast and gentle reagents and methods of single-cell cycling. In one embodiment, the disclosure provides ultra-fast clickable fluorophores and quenchers (FAST probes).

Reagents and Linkers

In some embodiments, the present disclosure provides a tridentate reagent comprising a click-reactive group (trans-cyclooctene, TCO) capable of undergoing a click reaction with a tetrazine (Tz) reagent comprising a fluorescence quencher, a fluorophore capable of being detected by fluorescent imaging, and a group reactive with a side chain of an amino acid of a protein. The tridentate reagent may be used to covalently modify a side chain of at least one amino acid of the protein. Hence, the covalently modified protein comprises a fluorophore (which makes the protein detectable by fluorescent imaging) and a TCO reactive group capable of undergoing a reaction with a tetrazine (Tz) reagent comprising a fluorescence quencher. Hence, the tridentate reagent may be used to covalently modify a protein simultaneously with a fluorophore and a fluorescent quencher, thereby rendering the protein undetectable by fluorescence imaging (the quencher absorbs the fluorescence from the fluorophore).

In some embodiments, the tridentate reagent, as well as the synthetic intermediates useful in preparing the tridentate reagent, are encompassed by the Formula (A):

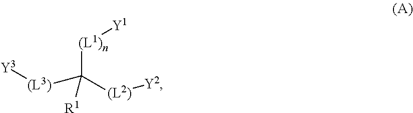

or a pharmaceutically acceptable salt thereof, wherein R¹, L¹, n, L², m, L³, p, Y¹, Y², and Y³ are as described herein.

In some embodiments, the tridentate reagent, as well as the synthetic intermediates useful in preparing the tridentate reagent, are encompassed by the Formula (I):

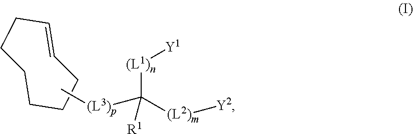

or a pharmaceutically acceptable salt thereof, wherein R¹, L¹, n, L², m, L³, Y¹ and Y² are as described herein.

In some embodiments of Formula (A):

R¹ is selected from H, halo, C₁₋₆ alkyl, C₁₋₆ haloalkyl, C₁₋₆ alkoxy, and C₁₋₆ haloalkoxy;

each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,

n is an integer from 1 to 10;

each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,

m is an integer from 1 to 10;

each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ, —(CH₂CH(CH₃)O)ₓ, and a moiety formed by a click reaction, wherein said C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ, and —(CH₂CH(CH₃)O)ₓ— are each optionally substituted with 1, 2, 3, 4, or 5 substituents independently selected from OH, NH₂, C₁₋₆ alkylamino, di(C₁₋₆-alkyl)amino, C₁₋₆ haloalkyl, C₁₋₆ alkoxy, C₁₋₆ haloalkoxy, and (L⁴)ₒ-Y³;

each L⁴ is independently selected from N(R^{N1}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,

p is an integer from 1 to 20;

o is an integer from 1 to 10;

each x is independently an integer from 1 to 2,000;

each R^{N} is independently selected from H, C₁₋₃ alkyl, C₁₋₃ haloalkyl, and (L⁴)ₒ-Y³;

each R^{N1} is independently selected from H, C₁₋₃ alkyl, and C₁₋₃ haloalkyl;

Y¹ is selected from NR^{c1}R^{1A}, OR², and C(═O)R³;

R^{1A} selected from H, an amine protecting group, and a residue of a fluorophore;

R² is selected from H, an alcohol protecting group, and a residue of a fluorophore;

R³ is selected from OR^{a1} and a residue of a fluorophore;

Y² is selected from C(═O)OR^{a1}, NR^{c1}R⁴, OR⁵; and a group reactive with a side chain of an amino acid of a protein;

R^{a1} is selected from H and a carboxylic acid protecting group;

R^{c1} is selected from H, C₁₋₃ alkyl, and C₁₋₃ haloalkyl;

R⁴ is selected from H and an amine protecting group;

R⁵ is selected from H and an alcohol protecting group; and

Y³ is a chemical group that is reactive in a biorthogonal chemical reaction.

In some embodiments of Formula (A) or Formula (I):
R¹ is selected from H, halo, C₁₋₆ alkyl, C₁₋₆ haloalkyl, C₁₋₆ alkoxy, and C₁₋₆ haloalkoxy;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
n is an integer from 1 to 10;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
m is an integer from 1 to 10;
each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
p is an integer from 1 to 10;
each x is independently an integer from 1 to 2,000;
each R^{N} is independently selected from H, C₁₋₃ alkyl, and C₁₋₃ haloalkyl;
Y¹ is selected from NR^{c1}R^{1A}, OR², and C(═O)R³;
R^{1A} selected from H, an amine protecting group, and a residue of a fluorophore;
R² is selected from H, an alcohol protecting group, and a residue of a fluorophore;
R³ is selected from OR^{a1} and a residue of a fluorophore;
Y² is selected from C(═O)OR^{a1}, NR^{c1}R⁴, OR⁵; and a group reactive with a side chain of an amino acid of a protein;
R^{a1} is selected from H and a carboxylic acid protecting group;
R^{c1} is selected from H, C₁₋₃ alkyl, and C₁₋₃ haloalkyl;
R⁴ is selected from H and an amine protecting group; and
R⁵ is selected from H an alcohol protecting group.

In some embodiments, R¹ is H. In some embodiments, R¹ is halo. In some embodiments, R¹ is C₁₋₆ alkyl. In some embodiments, R¹ is selected from H and halo. In some embodiments, R¹ is selected from H, halo, and C₁₋₆ alkyl.

In some embodiments, n is an integer from 1 to 7. In some embodiments, n is an integer from 1 to 5. In some embodiments, n is 1. In some embodiments, n is 2. In some embodiments, n is 3.

In some embodiments, each L¹ is independently selected from NH, O, C(═O), C₁₋₆ alkylene, C₆₋₁₀ arylene, —(OCH₂CH₂)ₓ—, and —(CH₂CH₂O)ₓ—.

In some embodiments, n is an integer from 1 to 5, and each L¹ is selected from NH, O, C(═O), C₁₋₆ alkylene, and C₆₋₁₀ arylene.

In some embodiments, n is 1 and L¹ is C₁₋₆ alkylene.

In some embodiments, m is an integer from 1 to 7. In some embodiments, m is an integer from 1 to 5. In some embodiments, m is at least 1. In some embodiments, m is an integer from 2 to 10. In some embodiments, m is an integer from 3 to 7.

In some embodiments, m is an integer from 1 to 5, and each L² is independently selected from NH, O, C(═O), C₁₋₆ alkylene, C₆₋₁₀ arylene, —(OCH₂CH₂)ₓ, —(CH₂CH₂O)ₓ, —(OCH(CH₃)CH₂)ₓ, and —(CH₂CH(CH₃)O)ₓ—.

In some embodiments, m is 4, and each L² is independently selected from NH, C(═O), C₁₋₆ alkylene, and —(OCH₂CH₂)ₓ—.

In some embodiments, p is an integer from 1 to 7. In some embodiments, p is an integer from 1 to 5. In some embodiments, p is at least 1. In some embodiments, p is an integer from 2 to 10. In some embodiments, p is an integer from 3 to 7.

In some embodiments, p is an integer from 1 to 15. In some embodiments, p is an integer from 1 to 10. In some embodiments, p is an integer from 1 to 7.

In some embodiments, each L³ is independently selected from N(R^{N}), 0, C(═O), C₁₋₆ alkylene, C₆₋₁₀ arylene, C₃₋₇ cycloalkylene, and a moiety formed by a click reaction, wherein said C₁₋₆ alkylene, C₃₋₇ cycloalkylene, and C₆₋₁₀ arylene are each optionally substituted with 1, 2, 3, or 4 substituents independently selected from OH and (L⁴)ₒ-Y³.

In some embodiments, the moiety formed by a click reaction is a reaction product of any one of the well-known click reactions, such as Huisgen cycloaddition (also known as [3+2] cycloaddition of alkynes and azides to form triazoles), Staudinger ligation (i.e., a reaction between an azide and a phosphine), a reaction of oxanorbornadienes and azides to from triazoles, an inverse-demand Diels-Alder reaction of tetrazines (e.g., dipyridyl tetrazines) and trans-cycloctynes, inverse-demand Diels-Alder reaction of tetrazines (e.g., monoaryl tetrazines) and norbornenes, a reaction of tetrazines and cyclopropenes, a reaction of cyclopropenes and nitrile imines, a photoinduced 1,3-dipolar cycloaddition of tetrazoles and alkenes, a 1,3-dipolar cycloaddition of nitrile oxides and norbornenes, a [4+1] cycloaddition isocyanides and tetrazines, or a 1,3-cycloaddition of nitrones and alkynes. In some embodiments, the moiety formed by a click reaction comprises a triazole. In some embodiments, the moiety formed by a click reaction is selected from:

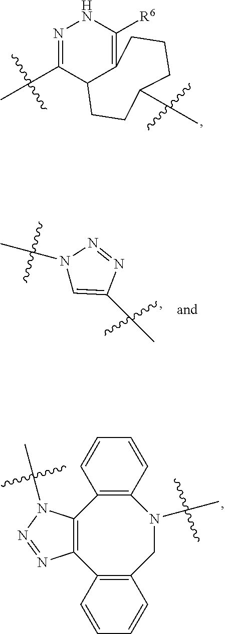

wherein R⁶ is selected from H and C₁₋₆ alkyl.

In some embodiments, at least one L³ is NR^{N}, and R^{N} is (L⁴)ₒ-Y³.

In some embodiments, each L³ is independently selected from NH, O, C(═O), C₁₋₆ alkylene, C₆₋₁₀ arylene, —(OCH₂CH₂)ₓ—, and —(CH₂CH₂O)ₓ—.

In some embodiments, p is an integer from 1 to 7, and each L³ is independently selected from NH, O, C(═O), C₁₋₆ alkylene, C₆₋₁₀ arylene, and —(OCH₂CH₂)ₓ. In some embodiments, p is 3, and each L³ is independently selected from NH, O, and C(═O).

In some embodiments, o is an integer from 1 to 7. In some embodiments, o is an integer from 1 to 5. In some embodiments, each L⁴ is independently selected from NH, O, C(═O), and C₁₋₆ alkylene. In some embodiments, each L⁴ is independently selected from NH, O, and C(═O).

In some embodiments, x is an integer from 2 to 10. In some embodiments, x is 3, 4, 5, or 6.

In some embodiments:

R¹ is H;

n is an integer from 1 to 5, and each L¹ is selected from NH, O, C(═O), C₁₋₆ alkylene, and C₆₋₁₀ arylene;

m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—;

x is an integer from 2 to 10;

p is an integer from 1 to 15;

each L³ is independently selected from N(R^{N}), O, C(═O), C₁₋₆ alkylene, C₆₋₁₀ arylene, C₃₋₇ cycloalkylene, and a moiety formed by a click reaction, wherein said C₁₋₆ alkylene, C₃₋₇ cycloalkylene, and C₆₋₁₀ arylene are each optionally substituted with 1, 2, 3, or 4 substituents independently selected from OH and (L⁴)ₒ-Y³;

each R^{N} is independently selected from H and (L⁴)ₒ-Y³;

o is an integer from 1 to 5; and

each L⁴ is independently selected from NH, O, C(═O), and C₁₋₆ alkylene.

In some embodiments:

R¹ is H;

n is an integer from 1 to 5, and each L¹ is selected from NH, O, C(═O), C₁₋₆ alkylene, and C₆₋₁₀ arylene;

m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—;

p is an integer from 1 to 7, and each L³ is independently selected from NH, O, C(═O), C₁₋₆ alkylene, C₆₋₁₀ arylene, and —(OCH₂CH₂)ₓ—; and

x is an integer from 2 to 10.

In some embodiments:

R¹ is H;

n is 1 and L¹ is C₁₋₆ alkylene;

m is 4, and each L² is independently selected from NH, C(═O), C₁₋₆ alkylene, and —(OCH₂CH₂)ₓ—;

p is 3, and each L³ is independently selected from NH, O, and C(═O); and

x is an integer from 2 to 10.

In some embodiments, Y³ comprises a chemical group selected from an azide (—N₃), an aliphatic alkyne (—C≡CH), a cyclooctyne, a cyclooctene, a cyclohexene, a nitrone, an isocyanide, a cyclopropene, a norborene, a diphenylphosphine, nitrile imine, a tetrazole, a nitrile oxide, and a tetrazine.

In some embodiments, Y³ comprises a chemical group selected from any one of the following groups:

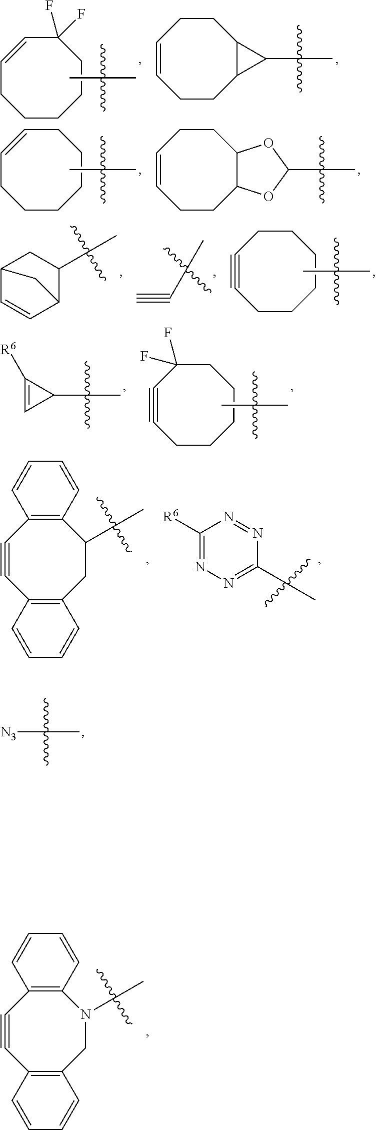

wherein R⁶ is selected from H and C₁₋₆ alkyl.

In some embodiments, Y³ comprises a chemical group selected from:

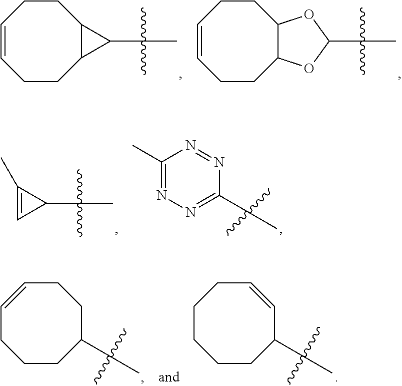

In some embodiments, Y³ comprises a chemical group selected from:

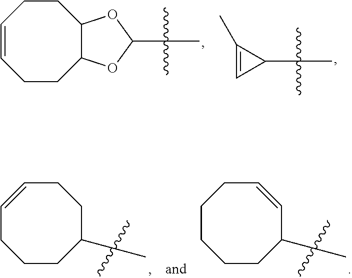

In some embodiments, Y³ comprises a chemical group selected from:

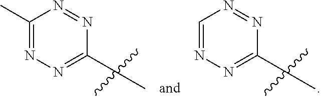

In some embodiments, the compound of Formula (A) has formula:

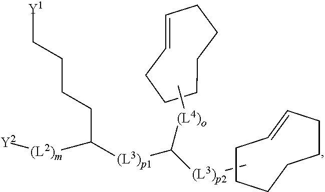

or a pharmaceutically acceptable salt thereof, wherein

the sum of p1 and p2 is less than p by at least 1.

In some embodiments, the compound of Formula (A) has formula:

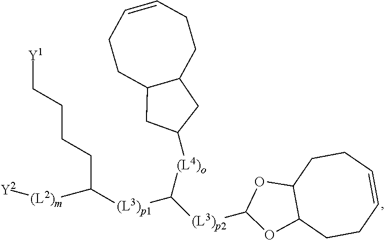

or a pharmaceutically acceptable salt thereof, wherein

the sum of p1 and p2 is less than p by at least 1.

In some embodiments, the compound of Formula (A) has formula:

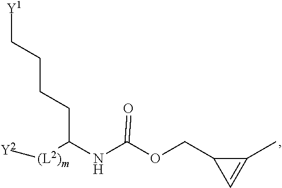

or a pharmaceutically acceptable salt thereof.

In some embodiments, the compound of Formula (A) has formula:

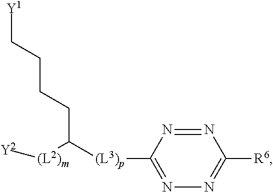

or a pharmaceutically acceptable salt thereof,

wherein R⁶ is selected from H and C₁₋₆ alkyl.

In some embodiments, the compound of Formula (A) has formula:

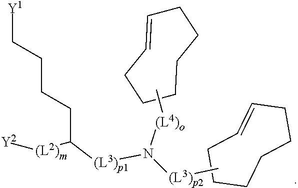

or a pharmaceutically acceptable salt thereof, wherein

the sum of p1 and p2 is less than p by at least 1.

In some embodiments, the compound of Formula (I) has formula:

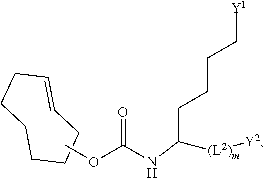

or a pharmaceutically acceptable salt thereof.

In some embodiments, the compound of Formula (I) has formula:

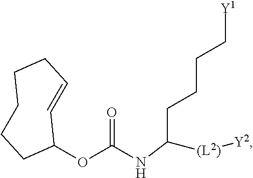

or a pharmaceutically acceptable salt thereof.

In some embodiments, the compound of Formula (I) has formula:

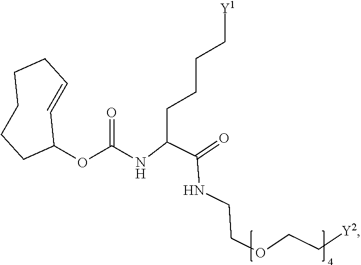

or a pharmaceutically acceptable salt thereof.

In some embodiments, the compound of Formula (I) has formula:

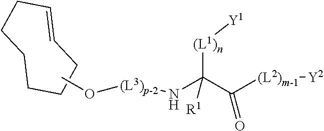

or a pharmaceutically acceptable salt thereof.

In some embodiments, (L¹)ₙ comprises a side chain of an amino acid (e.g., lysine, serine, threonine, cysteine, tyrosine, aspartic acid, or glutamic acid), and Y¹ comprises the terminal functional group of the side chain of the amino acid (e.g., N, O, S, or C(═O)).

In some embodiments, Y¹ is NHR^{1A}.

In some embodiments, R^{1A} is a residue of a fluorophore.

In some embodiments, R^{1A} is an amine protecting group.

In some embodiments, Y¹ is NH₂.

In some embodiments, Y¹ is OR².

In some embodiments, Y¹ is OH.

In some embodiments, R² is an alcohol protecting group.

In some embodiments, R² is a residue of a fluorophore.

In some embodiments, Y¹ is C(═O)R³.

In some embodiments, Y¹ is C(═O)OH.

In some embodiments, R³ is OR^{a1}, and R^{a1} is a carboxylic acid protecting group.

In some embodiments, R³ is a residue of a fluorophore.

In some embodiments, Y² is C(═O)OR^{a1}In some embodiments, Y² is C(═O)OH.

In some embodiments, R^{a1} is a carboxylic acid protecting group.

In some embodiments, Y² is NHR⁴.

In some embodiments, Y² is NH₂.

In some embodiments, R⁴ is an amine-protecting group.

In some embodiments, Y² is OR⁵.

In some embodiments, Y² is OH.

In some embodiments, R⁵ is an alcohol-protecting group.

In some embodiments, Y² is a group reactive with a side chain of an amino acid of a protein. In some embodiments, the group reactive with a side chain of an amino acid of a protein is an activated ester group.

In some embodiments:

Y¹ is NHR^{1A}; and

Y² is selected from C(═O)OR^{a1} and a group reactive with a side chain of an amino acid of a protein.

In some embodiments:

Y¹ is NH₂; and

Y² is C(═O)OH.

In some embodiments:

Y¹ is NHR^{1A}.

R¹ is an amine protecting group; and

Y² is C(═O)OH.

In some embodiments:

Y¹ is NHR^{1A}.

R¹ is a residue of a fluorophore; and

Y² is C(═O)OH.

In some embodiments:

Y¹ is NHR^{1A}.

R¹ is a residue of a fluorophore;

Y² is C(═O)OR^{a1}; and

R^{a1} is a carboxylic acid protecting group.

In some embodiments:

Y¹ is NHR^{1A}.

R¹ is a residue of a fluorophore; and

Y² is a group reactive with a side chain of an amino acid of a protein.

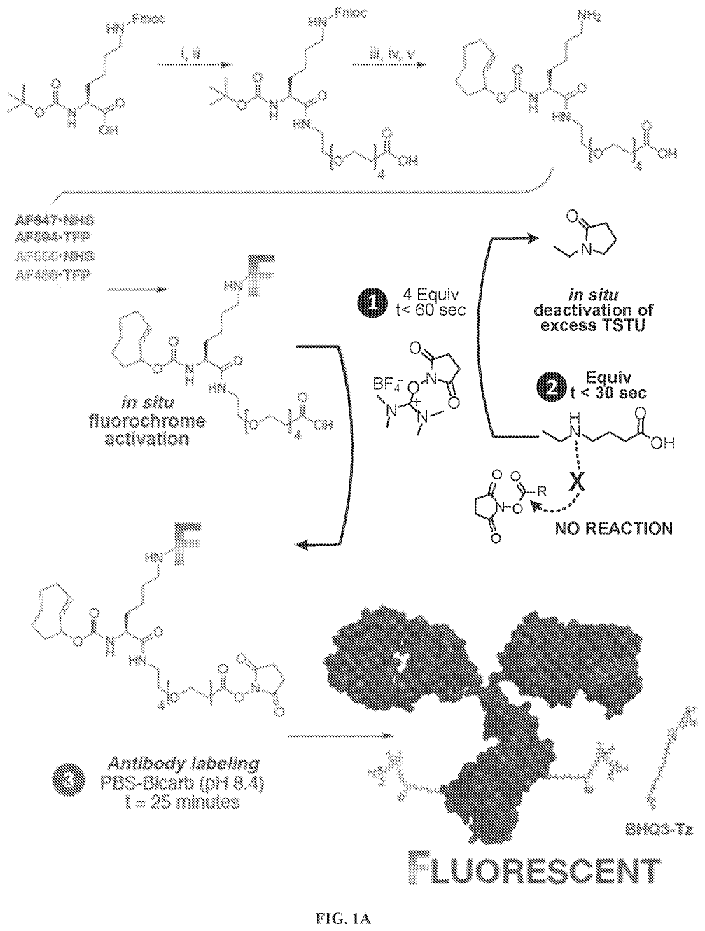

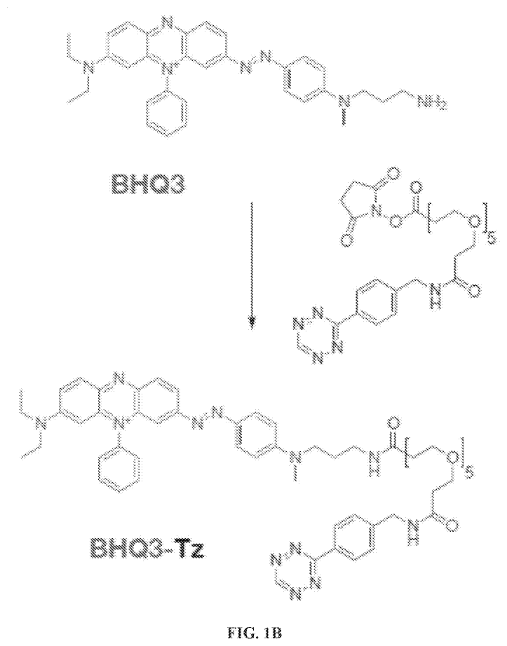

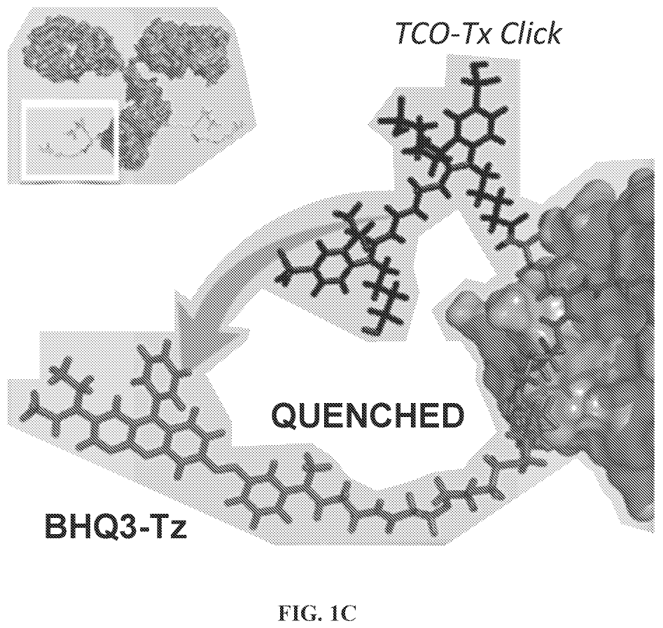

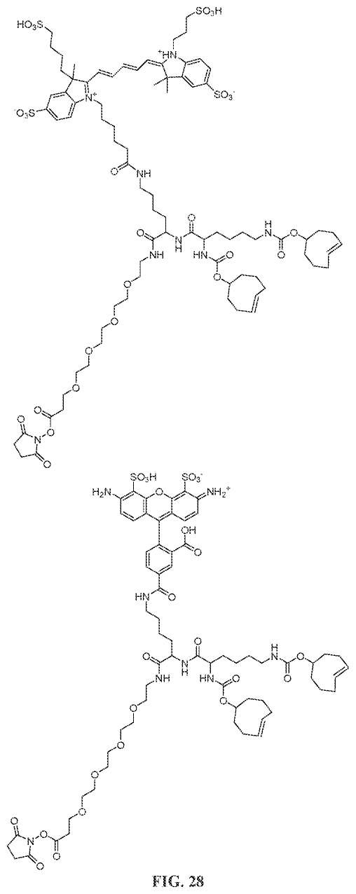

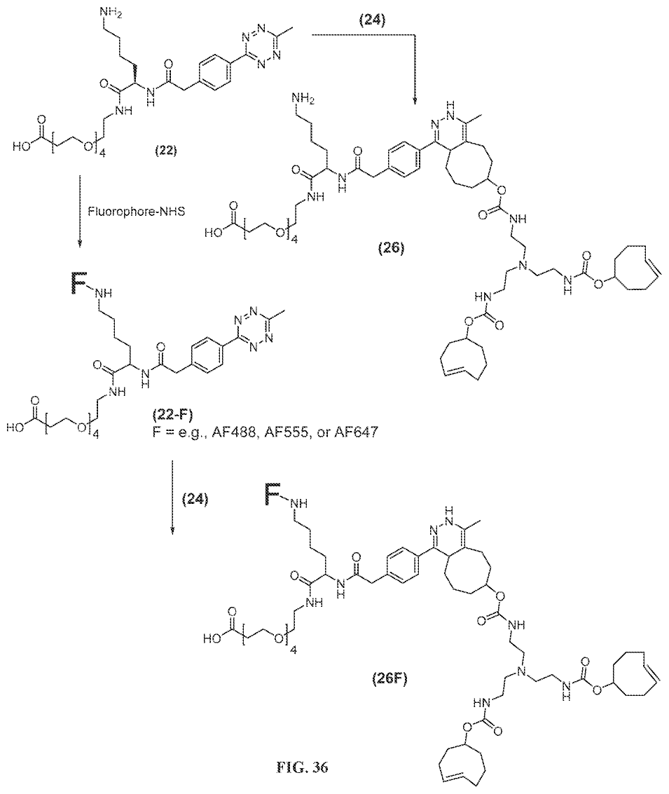

In some embodiments, the compound of Formula (A) is selected from any one of the compounds depicted in FIGS. 1A, 15, 16A, 16B, 22A, 22B, 23-33, 35, and 36, and described in the examples 1, 3, 9, 10, 11, 12, and 13, or a pharmaceutically acceptable salt thereof.

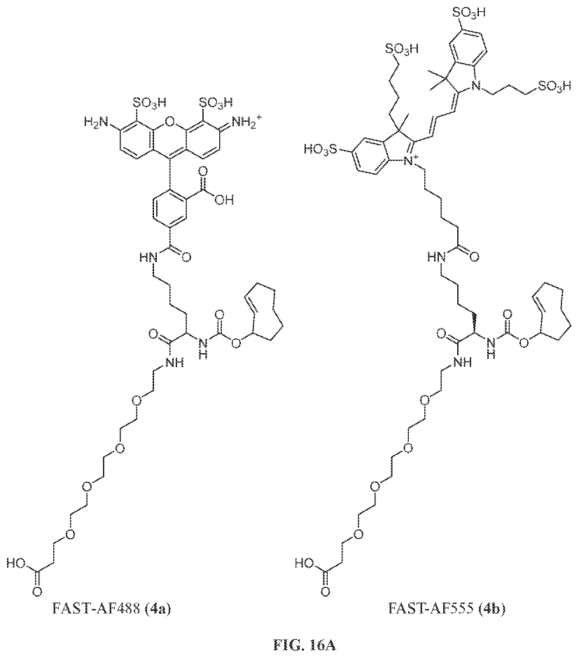

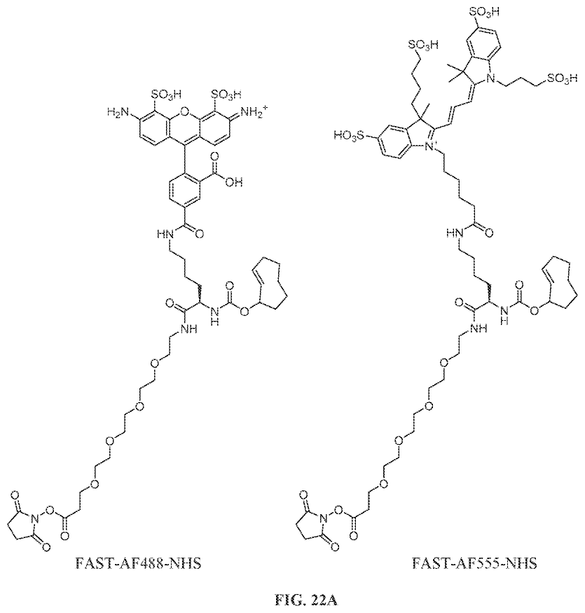

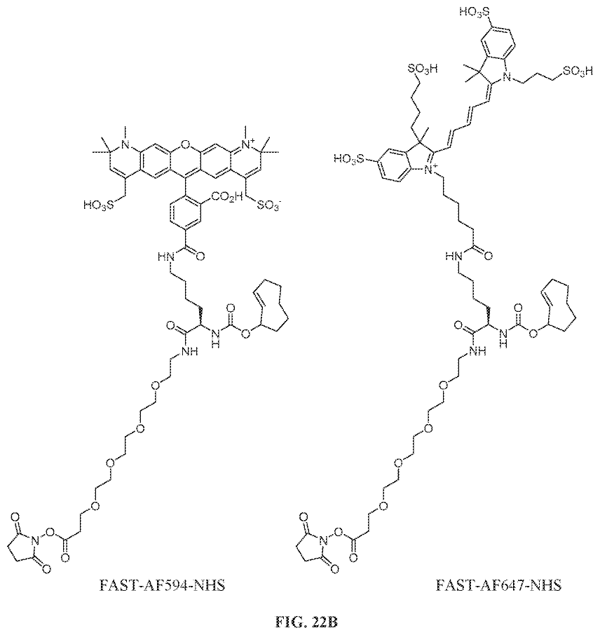

In some embodiments, the compound of Formula (I) is anyone of the compounds depicted in FIGS. 16A, 16B, 22A, and 22B, or a pharmaceutically acceptable salt thereof.

In some embodiments, a skilled chemist would be able to select and implement any of the amine protecting groups, alcohol protecting groups, or carboxylic acid protecting groups of the present disclosure. The chemistry of protecting groups can be found, for example, in P. G. M. Wuts and T. W. Greene, Protective Groups in Organic Synthesis, 4^{th} Ed., Wiley & Sons, Inc., New York (2006) (which is incorporated herein by reference), including suitable examples of the protecting groups, and methods for protection and deprotection, and the selection of appropriate protecting groups.

Suitable examples of amine-protecting groups include Carbobenzyloxy (Cbz) group, p-Methoxybenzyl carbonyl (Moz or MeOZ), tert-Butyloxycarbonyl (BOC) group, 9-Fluorenylmethyloxycarbonyl (Fmoc), Acetyl (Ac), Benzoyl (Bz), Benzyl (Bn) group, Carbamate group, p-Methoxybenzyl (PMB), 3,4-Dimethoxybenzyl (DMPM), p-Methoxyphenyl (PMP) group, Tosyl (Ts) group, Troc (trichloroethyl chloroformate), and nosyl group.

Suitable examples of alcohol-protecting groups include Acetyl (Ac), Benzoyl (Bz), Benzyl (Bn), β-Methoxyethoxymethyl ether (MEM), Dimethoxytrityl, [bis-(4-methoxyphenyl)phenylmethyl] (DMT), Methoxymethyl ether (MOM), Methoxytrityl [(4-methoxyphenyl)diphenylmethyl] (MMT), p-Methoxybenzyl ether (PMB), Methylthiomethyl ether, Pivaloyl (Piv), Tetrahydropyranyl (THP), Tetrahydrofuran (THF), Trityl (triphenylmethyl, Tr), Silyl ether (most popular ones include trimethylsilyl (TMS), tert-butyldimethylsilyl (TBDMS), tri-iso-propylsilyloxymethyl (TOM), and triisopropylsilyl (TIPS) ethers), Methyl ethers, and Ethoxyethyl ethers (EE).

Suitable examples of carboxylic acid protecting groups include methyl esters, benzyl esters, tert-butyl esters, esters of 2,6-disubstituted phenols (e.g., 2,6-dimethylphenol, 2,6-diisopropylphenol, 2,6-di-tert-butylphenol), silyl esters, orthoesters, and oxazoline.

Suitable examples of groups reactive with a side chain of an amino acid of a protein are described, for example, in D. Shannon, Covalent protein modification: the current landscape of residue-specific electrophiles, Current Opinion in Chemical Biology 2015, 24, 18-26, which is incorporated herein by reference in its entirety.

Suitable examples of groups reactive with OH of a serine include the following groups:

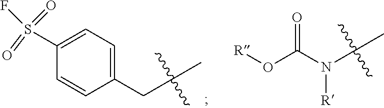

(R′ is H or C₁₋₃ alkyl, R″ is C₁₋₃ alkyl).

Suitable examples of groups reactive with SH of a cysteine include the following groups:

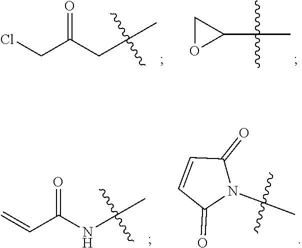

Suitable example of groups reactive with NH₂ of a lysine includes an activated ester of formula:

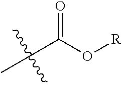

(R is, e.g., N-succinimidyl, N-benzotriazolyl, 4-nitrophenyl, or pentafluorophenyl).

Suitable examples of fluorophores include any fluorescent chemical compound that can re-emit light upon light excitation. The fluorophores can by excited by a light of a wavelength form about 300 nm to about 800 nm, and then emit light of a wavelength from about 350 nm to about 770 nm (e.g., violet, blue, cyan, green, yellow, orange or red light), which can be detected by fluorescent imaging devices, including the ability to measure the intensity of the fluorescence. Suitable examples of fluorophores include AF488, Hydroxycoumarin blue, methoxycoumarin blue, Alexa fluor blue, aminocoumarin blue, Cy2 green (dark), FAM green (dark), Alexa fluor 488 green (light), Fluorescein FITC green (light), Alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red. Absorbance and emission wavelengths of these fluorophores are well known in the art.

In some embodiments, a salt of a compound of Formula (A) or Formula (I) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of Formula (A) or Formula (I) include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2- sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of Formula (A) or Formula (I) include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine;

pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the present disclosure also provides a linker of Formula:

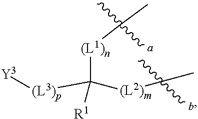

wherein a designates a point of attachment of the linker to a fluorophore, b designates a point of attachment to a protein (e.g., antibody), and Y³, L¹, n, L², m, p, and R¹ are as described herein for Formula (A).

In some embodiments, the present disclosure also provides a linker of Formula.

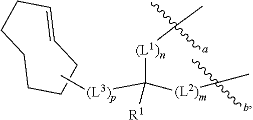

wherein a designates a point of attachment of the linker to a fluorophore, b designates a point of attachment to a protein (e.g., antibody), and L¹, n, L², m, L³, p, and R¹ are as described herein for Formula (I).

Protein Conjugates

In some embodiments, the tridentate reagents of Formula (A) can be reacted with a protein to obtain a protein conjugate of Formula (B):

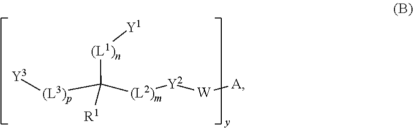

or a pharmaceutically acceptable salt thereof, wherein R¹, L¹, n, L², m, L³, p, Y¹, Y², Y³, W, A, and y are as described herein. In some embodiments, R¹, L¹, n, L² m, L³, p, Y¹, and Y³ are as described herein for Formula (A).

In some embodiments, the tridentate reagents of Formula (I) can be reacted with a protein to obtain a protein conjugate of Formula (II):

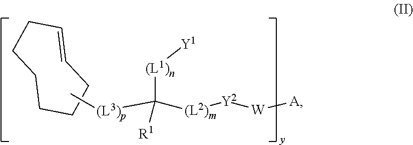

or a pharmaceutically acceptable salt thereof, wherein R¹, L¹, n, L², m, L³, p, Y¹, Y², W, A, and y are as described herein. In some embodiments, R¹, L¹, n, L², m, L, p, and Y¹ are as described herein for Formula (I).

In some embodiments of Formula (B):

A is a residue of a protein;

y is an integer from 1 to 10;

R¹ is selected from H, halo, C₁₋₆ alkyl, C₁₋₆ haloalkyl, C₁₋₆ alkoxy, and C₁₋₆ haloalkoxy;

each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,

n is an integer from 1 to 10;

each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,

m is an integer from 1 to 10;

each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ, —(CH₂CH(CH₃)O)ₓ, and a moiety formed by a click reaction, wherein said C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ, and —(CH₂CH(CH₃)O)ₓ— are each optionally substituted with 1, 2, 3, 4, or 5 substituents independently selected from OH, NH₂, C₁₋₆ alkylamino, di(C₁₋₆-alkyl)amino, C₁₋₆ haloalkyl, C₁₋₆ alkoxy, C₁₋₆ haloalkoxy, and (L⁴)ₒ-Y³;

each L⁴ is independently selected from N(R^{N1}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—, p is an integer from 1 to 20;

o is an integer from 1 to 10;

each x is independently an integer from 1 to 2,000;

each R^{N} is independently selected from H, C₁₋₃ alkyl, C₁₋₃ haloalkyl, and (L⁴)ₒ-Y³ each R^{N1} is independently selected from H, C₁₋₃ alkyl, and C₁₋₃ haloalkyl;

Y¹ is selected from NR^{c1}R^{1A}, OR², and C(═O)R³;

R^{1A}, R², and R³ are each independently a residue of a fluorophore;

R¹ is selected from H, C₁₋₃ alkyl, and C₁₋₃ haloalkyl;

each W is selected from:

(i) O of a side chain of serine, threonine, or tyrosine of the protein A;

(ii) S of a side chain of cysteine of the protein A;

(iii) NH of a side chain of lysine of the protein A; and

(iv) C(═O) of a side chain of aspartic acid or glutamic acid of the protein A;

Y² is a residue of a group which, prior to conjugation with the protein A, was a group reactive with a side chain of an amino acid of the protein A; and

Y³ is a chemical group that is reactive in a biorthogonal chemical reaction.

In some embodiments of Formula (B) or Formula (II):

A is a residue of a protein;

y is an integer from 1 to 10;

R¹ is selected from H, halo, C₁₋₆ alkyl, C₁₋₆ haloalkyl, C₁₋₆ alkoxy, and C₁₋₆ haloalkoxy;

each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,

n is an integer from 1 to 10;

each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,

m is an integer from 1 to 10;

each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—, p is an integer from 1 to 10;

each x is independently an integer from 1 to 2,000;

each R^{N} is independently selected from H, C₁₋₃ alkyl, and C₁₋₃ haloalkyl;

Y¹ is selected from NR^{c1}R^{1A}, OR², and C(═O)R³;

R^{1A}, R², and R³ are each independently a residue of a fluorophore;

R¹ is selected from H, C₁₋₃ alkyl, and C₁₋₃ haloalkyl;

each W is selected from:
(i) O of a side chain of serine, threonine, or tyrosine of the protein A;
(ii) S of a side chain of cysteine of the protein A;
(iii) NH of a side chain of lysine of the protein A; and
(iv) C(═O) of a side chain of aspartic acid or glutamic acid of the protein A;

Y² is a residue of a group which, prior to conjugation with the protein A, was a group reactive with a side chain of an amino acid of the protein A.

In some embodiments, R¹ is H. In some embodiments, R¹ is halo. In some embodiments, R¹ is C₁₋₆ alkyl. In some embodiments, R¹ is selected from H and halo. In some embodiments, R¹ is selected from H, halo, and C₁₋₆ alkyl.

In some embodiments, n is an integer from 1 to 7. In some embodiments, n is an integer from 1 to 5. In some embodiments, n is 1. In some embodiments, n is 2. In some embodiments, n is 3.

In some embodiments, each L¹ is independently selected from NH, O, C(═O), C₁₋₆ alkylene, C₆₋₁₀ arylene, —(OCH₂CH₂)ₓ—, and —(CH₂CH₂O)ₓ—.

In some embodiments, n is an integer from 1 to 5, and each L¹ is selected from NH, O, C(═O), C₁₋₆ alkylene, and C₆₋₁₀ arylene.

In some embodiments, n is 1 and L¹ is C₁₋₆ alkylene.

In some embodiments, m is an integer from 1 to 7. In some embodiments, m is an integer from 1 to 5. In some embodiments, m is at least 1. In some embodiments,

m is an integer from 2 to 10. In some embodiments, m is an integer from 3 to 7.

In some embodiments, each L² is independently selected from NH, O, C(═O), C₁₋₆ alkylene, C₆₋₁₀ arylene, —(OCH₂CH₂)ₓ—, and —(CH₂CH₂O)ₓ—.

In some embodiments, m is an integer from 1 to 5, and each L² is independently selected from NH, O, C(═O), C₁₋₆ alkylene, C₆₋₁₀ arylene, —(OCH₂CH₂)ₓ, —(CH₂CH₂O)ₓ, —(OCH(CH₃)CH₂)ₓ, and —(CH₂CH(CH₃)O)ₓ—.

In some embodiments, m is 4, and each L² is independently selected from NH, C(═O), C₁₋₆ alkylene, and —(OCH₂CH₂)ₓ—.

In some embodiments, p is an integer from 1 to 15. In some embodiments, p is an integer from 1 to 7. In some embodiments, p is an integer from 1 to 5. In some embodiments, p is at least 1. In some embodiments, p is an integer from 2 to 10. In some embodiments, p is an integer from 3 to 7.

In some embodiments, each L³ is independently selected from N(R^{N}), 0, C(═O), C₁₋₆ alkylene, C₆₋₁₀ arylene, C₃₋₇ cycloalkylene, and a moiety formed by a click reaction, wherein said C₁₋₆ alkylene, C₃₋₇ cycloalkylene, and C₆₋₁₀ arylene are each optionally substituted with 1, 2, 3, or 4 substituents independently selected from OH and (L⁴)ₒ-Y³.

In some embodiments, each L³ is independently selected from NH, O, C(═O), C₁₋₆ alkylene, C₆₋₁₀ arylene, —(OCH₂CH₂)ₓ—, and —(CH₂CH₂O)ₓ—.

In some embodiments, p is an integer from 1 to 7, and each L³ is independently selected from NH, O, C(═O), C₁₋₆ alkylene, C₆₋₁₀ arylene, and —(OCH₂CH₂)ₓ.

In some embodiments, p is 3, and each L³ is independently selected from NH, O, and C(═O).

In some embodiments, o is an integer from 1 to 5. In some embodiments, each L⁴ is independently selected from NH, O, C(═O), and C₁₋₆ alkylene.

In some embodiments, x is an integer from 2 to 10. In some embodiments, x is 3, 4, 5, or 6.

In some embodiments:

R¹ is H;

n is an integer from 1 to 5, and each L¹ is selected from NH, O, C(═O), C₁₋₆ alkylene, and C₆₋₁₀ arylene;

m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—;

x is an integer from 2 to 10;

p is an integer from 1 to 15;

each L³ is independently selected from N(R^{N}), O, C(═O), C₁₋₆ alkylene, C₆₋₁₀ arylene, C₃₋₇ cycloalkylene, and a moiety formed by a click reaction, wherein said C₁₋₆ alkylene, C₃₋₇ cycloalkylene, and C₆₋₁₀ arylene are each optionally substituted with 1, 2, 3, or 4 substituents independently selected from OH and (L⁴)ₒ-Y³;

each R^{N} is independently selected from H and (L⁴)ₒ-Y³;

o is an integer from 1 to 5; and

each L⁴ is independently selected from NH, O, C(═O), and C₁₋₆ alkylene.

In some embodiments:

R¹ is H;

n is an integer from 1 to 5, and each L¹ is selected from NH, O, C(═O), C₁₋₆ alkylene, and C₆₋₁₀ arylene;

m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—;

p is an integer from 1 to 7, and each L³ is independently selected from NH, O, C(═O), C₁₋₆ alkylene, C₆₋₁₀ arylene, and —(OCH₂CH₂)ₓ—; and

x is an integer from 2 to 10.

In some embodiments:

R¹ is H;

n is 1 and L¹ is C₁₋₆ alkylene;

m is 4, and each L² is independently selected from NH, C(═O), C₁₋₆ alkylene, and —(OCH₂CH₂)ₓ—;

p is 3, and each L³ is independently selected from NH, O, and C(═O); and

x is an integer from 2 to 10.

In some embodiments, Y³ comprises a chemical group selected from an azide (—N₃), an aliphatic alkyne (—C≡CH), a cyclooctyne, a cyclooctene, a cyclohexene, a nitrone, an isocyanide, a cyclopropene, a norborene, a diphenylphosphine, nitrile imine, a tetrazole, a nitrile oxide, and a tetrazine.

In some embodiments, Y³ comprises a chemical group selected from any one of the following groups:

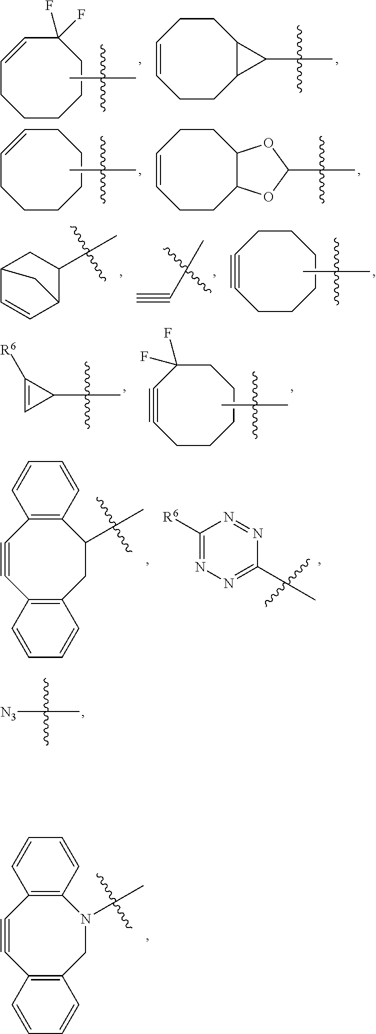

wherein R⁶ is selected from H and C₁₋₆ alkyl.

In some embodiments, Y³ comprises a chemical group selected from:

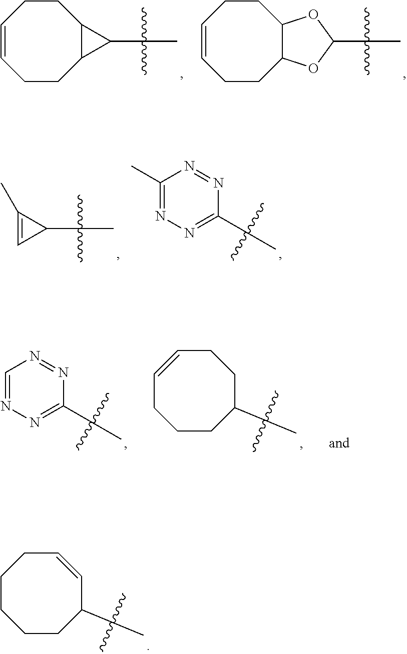

In some embodiments, Y³ comprises a chemical group selected from:

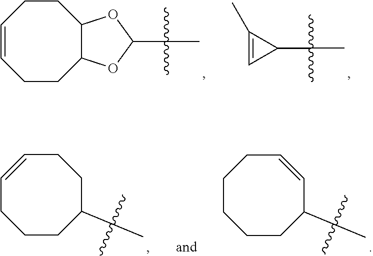

In some embodiments, Y³ comprises a chemical group selected from:

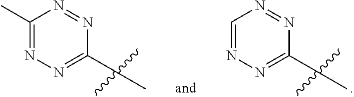

In some embodiments, the conjugate of Formula (B) has formula:

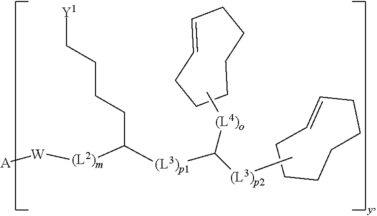

or a pharmaceutically acceptable salt thereof, wherein

the sum of p1 and p2 is less than p by at least 1.

In some embodiments, the conjugate of Formula (B) has formula:

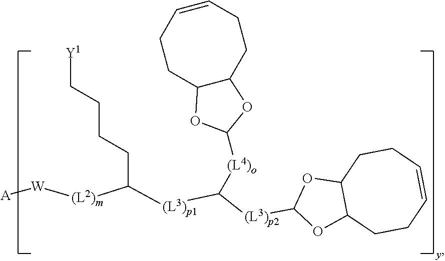

or a pharmaceutically acceptable salt thereof, wherein

the sum of p1 and p2 is less than p by at least 1.

In some embodiments, the conjugate of Formula (B) has formula:

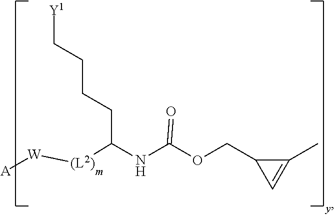

or a pharmaceutically acceptable salt thereof.

In some embodiments, the conjugate of Formula (B) has formula:

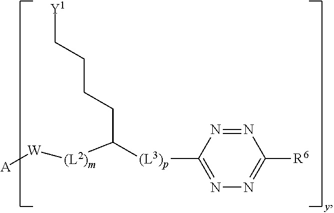

or a pharmaceutically acceptable salt thereof, wherein R⁶ is selected from H and C₁₋₆ alkyl.

In some embodiments, the conjugate of Formula (B) has formula:

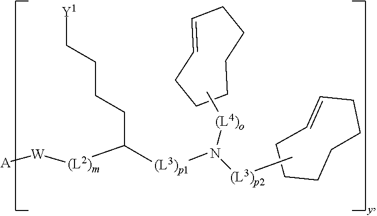

or a pharmaceutically acceptable salt thereof, wherein

the sum of p1 and p2 is less than p by at least 1.

In some embodiments, the compound of Formula (II) has formula:

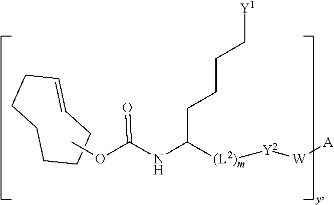

or a pharmaceutically acceptable salt thereof.

In some embodiments, the compound of Formula (II) has formula:

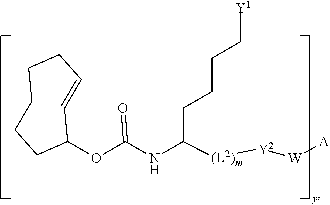

or a pharmaceutically acceptable salt thereof.

In some embodiments, the compound of Formula (II) has formula:

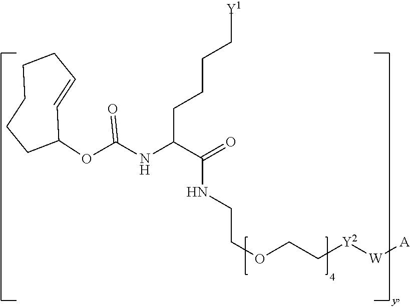

or a pharmaceutically acceptable salt thereof.

In some embodiments, the compound of Formula (II) has formula:

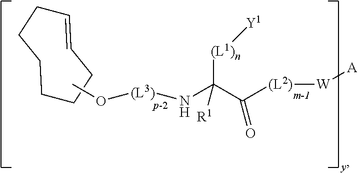

or a pharmaceutically acceptable salt thereof.

In some embodiments, (L¹)ₙ comprises a side chain of an amino acid (e.g., lysine, serine, threonine, cysteine, tyrosine, aspartic acid, or glutamic acid), and Y¹ comprises the terminal functional group of the side chain of the amino acid (e.g., N, O, S, or C(═O)).

In some embodiments, y is an integer from 4 to 6. In some embodiments, y is an integer from 1 to 10. In some embodiments, y is 1. In some embodiments, y is 4. In some embodiments, y is 5. In some embodiments, y is 6. In some embodiments, y is 7.

In some embodiments, Y¹ is NHR^{1A}

In some embodiments, Y¹ is OR².

In some embodiments, Y¹ is C(═O)R³.

The fluorophore in any one of the R^{1A}, R², and R³ can be any one of the fluorophores described herein for Formula (I). In some embodiments, the fluorophore of Formula (II) is selected from AF488, AF647, AF594, and AF555.

In some embodiments, Y², prior to conjugation to protein A, is any one of the reactive Y² groups described herein for Formula (I). Suitable examples of Y² groups of Formula (II) include C(═O), and any one of the following moieties:

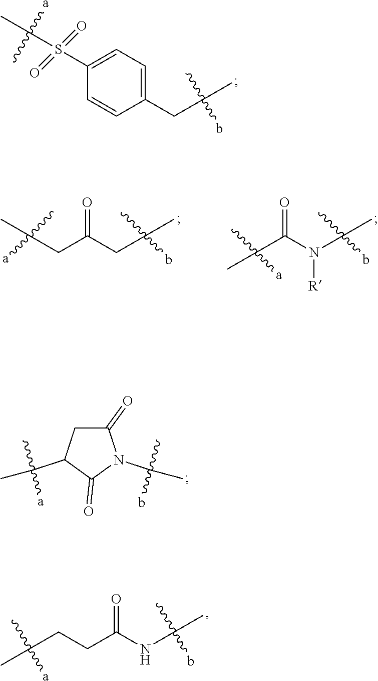

wherein a is point of attachment of the moiety to W, and b is a point of attachment of the moiety to L².

In some embodiments, W is O of a side chain of serine, threonine, or tyrosine of the protein A. In some embodiments, W is S of a side chain of cysteine of the protein A. In some embodiments, W is NH of a side chain of lysine of the protein A.

In some embodiments, W is C(═O) of a side chain of aspartic acid or glutamic acid of the protein A.

In some embodiments, each Y² is C(═O) and each W is NH of a side chain of lysine of the protein A. In some embodiments, each Y² is C(═O) and at least one W is S of a side chain of cysteine of the protein A.

In some embodiments, the protein is selected from an antibody, an antibody fragment, an engineered antibody, a peptide, and an aptamer. In some embodiments, the protein is an antibody. In some embodiments, the antibody is specific to an antigen which is a biomarker of a disease or condition. In some embodiments, the disease or condition is cancer. In some embodiments, the disease or conditions is a disease of the immune system. Suitable examples of such diseases include severe combined immunodeficiency (SCID), autoimmune disorder, familial Mediterranean fever and Crohn's disease (inflammatory bowel disease), arthritis (including rheumatoid arthritis), Hashimoto's thyroiditis, diabetes mellitus type 1, systemic lupus erythematosus, and myasthenia gravis. In some embodiments, the antigen is a biomarker of immune system response to a viral infection or a vaccine. Suitable example of viral infections include infections caused by a DNA virus, an RNA virus, or a coronavirus. One example of a viral infection is influenza. Another example of a viral infection is a coronavirus infection, such as COVID-19 (caused by SARS-CoV-2), Middle East respiratory syndrome (MERS) (caused by MERS-CoV), or severe acute respiratory syndrome (SARS) (caused by SARS-CoV). In some embodiments, the antigen is a biomarker of a cytokine storm. A cytokine storm can occur as a result of an infection (e.g., a viral infection as described herein), a vaccine (e.g., a vaccine against any of the viral infections described herein), an autoimmune condition, or other disease. Suitable examples of such cytokines include pro-inflammatory cytokines such as IL-6, IL-1, TNF-α, or interferon. In some embodiments, the antibody is specific to an antigen indicative of an immune system response to COVID-19 (including cytokine storm).

Suitable examples of biomarkers include CD45, CD3, CD4, CD8, PD-1, PD-L¹, CD11b, F4/80, CD163, CD206, Ly6G, CD11c, and MHCII. Any other biomarker the presence of which in the cell (e.g., on the cell surface) is known in the art to be indicative of severity of the disease, or to be indicative of the presence of some disease state, can be used as an antigen for the antibody A of the Formula (B) or Formula (II). Some examples of cancer biomarkers include alpha fetoprotein (AFP), CA15-3, CA27-29, CA19-9, CA-125, calcitonin, calretinin, carcinoembryonic antigen, CD34, CD99MIC 2, CD117, chromogranin, chromosomes 3, 7, 17, and 9p21, cytokeratin (various types: TPA, TPS, Cyfra21-1), desmin, epithelial membrane antigen (EMA), factor VIII, CD31 FL1, glial fibrillary acidic protein (GFAP), gross cystic disease fluid protein (GCDFP-15), HMB-45, human chorionic gonadotropin (hCG), immunoglobulin, inhibin, keratin (various types), lymphocyte marker (various types, MART-1 (Melan-A), myo D1, muscle-specific actin (MSA), neurofilament, neuron-specific enolase (NSE), placental alkaline phosphatase (PLAP), prostate-specific antigen (PSA), PTPRC (CD45), S100 protein, smooth muscle actin (SMA), synaptophysin, thymidine kinase, thyroglobulin (Tg), thyroid transcription factor-1 (TTF-1), tumor M2-PK, and vimentin.

In some embodiments, the biomarker is selected from CD45, CD3, CD8, CD4, FoxP3, NK1.1, CD19, CD20, CD11b, F4/80, CD11c, Ly6G, Ly6C, MHCII, PD-1, PD-L1, granzyme B, IFNγ, CK5/6, p16, CD56, CD68, CD14, CD1a, CD66b, CD39, TCF1, IL-120, and CD163. In some embodiments, the antibody is specific to PD-1 (e.g., pembrolizumab, nivolumab, or cemiplimab). In some embodiments, the antibody is specific to PD-L1 (e.g., atezolizumab, avelumab, or durvalumab).

In some embodiments, the present disclosure provides a composition comprising a protein conjugate of Formula (B) or Formula (II), or a pharmaceutically acceptable salt thereof, and an inert carrier. In some embodiments, the composition is an aqueous solution (i.e., the inert carrier is water). The aqueous solution may be a buffer, such as any buffer containing inert carrier such as water, phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, or any combination thereof. Some examples of buffers include Dulbecco's phosphate-buffered saline (DPBS), phosphate buffered saline, and Krebs-Henseleit Buffer. The pH of the buffer may be from about 5 to about 9, for example pH may be 6-8. The compound of Formula (A) or Formula (I), or a salt thereof, wherein Y² is a group reactive with a protein, may be admixed with the protein (e.g., antibody) in any of the aqueous solutions described here to obtain the compound of Formula (B) or Formula (II).

A composition (e.g., an aqueous solution) comprising the compound of Formula (B) or Formula (II), may be used to treat a cell (e.g., a cell containing a biomarker) to image the cell using the fluorophore of the Formula (B) or Formula (II).

Methods of Cellular Analysis

Accordingly, the present disclosure provides a method of examining a cell or a component of a cell (e.g., nucleus of a cell), the method comprising:

(i) contacting the cell with a conjugate of Formula (B) or Formula (II) comprising the residue of the fluorophore, or a pharmaceutically acceptable salt thereof, or a pharmaceutical composition comprising same;

(ii) imaging the cell with an imaging technique; and

(iii) after (ii), contacting the cell with a compound of Formula (C):

Y⁴-(L⁴)ₐ-Q (C),

or a pharmaceutically acceptable salt thereof, wherein Y⁴, L⁴, a, and Q are as described herein.

In some embodiments, the step (iii) comprises contacting the cell with a compound of Formula (III):

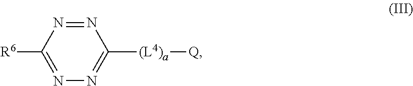

or a pharmaceutically acceptable salt thereof, wherein R⁶, L⁴, a, and Q are as described herein.

In some embodiments, the method comprises:
(i) contacting the cell with a conjugate of Formula (II) comprising the residue of the fluorophore, or a pharmaceutically acceptable salt thereof;
(ii) imaging the cell with an imaging technique; and
(iii) after (ii), contacting the cell with a compound of Formula (III):

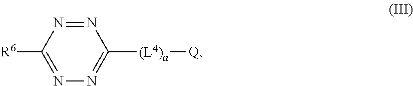

or a pharmaceutically acceptable salt thereof, wherein R⁶, L⁴, a, and Q are as described herein.

Without being bound by a theory, it is believed that when the cell is contacted with the protein conjugate of Formula (B) or Formula (II), the protein A (e.g., antibody) binds to its antigen on the surface of the cell or in the cytoplasm of the cell, and, therefore, the cell can be imaged by detecting fluorescence of the fluorophore in the Formula (B) or Formula (II).

In some embodiments, the imaging technique of step (ii) is a fluorescence imaging, such as microscopy, imaging probes, and spectroscopy. The fluorescence imaging devices include an excitation source, the emitted light collection source, optionally optical filters, and a means for visualization (e.g., a digital camera for taking fluorescence imaging photographs). Suitable examples of fluorescence imaging include internal reflection fluorescence microscopy, light sheet fluorescence microscopy, and fluorescence-lifetime imaging microscopy. Suitable imaging techniques are described, for example, in Rao, J. et al., Fluorescence imaging in vivo: recent advances, Current Opinion in Biotechnology, 18, (1), 2007, 17-25, which is incorporated herein by reference in its entirety.

In some embodiments of the Formula (C):

R⁶ is selected from H, C₁₋₆ alkyl, and C₁₋₆ haloalkyl;

each L⁴ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)_{y}—, —(CH₂CH₂O)_{y}—, —(OCH(CH₃)CH₂)_{y}—, and —(CH₂CH(CH₃)O)_{y}—,

a is an integer from 1 to 10;

each R^{N} is selected from H and C₁₋₃ alkyl;

each y is an integer from 1 to 2,000;

Q is a residue of a quencher; and

Y⁴ is a chemical group that is reactive in a biorthogonal chemical reaction, wherein the contacting of step (iii) results in decrease of the fluorescence of the fluorophore in the conjugate of Formula (B) or Formula (II), or a pharmaceutically acceptable salt thereof.

In some embodiments of the Formula (C) or Formula (III):

R⁶ is selected from H, C₁₋₆ alkyl, and C₁₋₆ haloalkyl;

each L⁴ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)_{y}—, —(CH₂CH₂O)_{y}—, —(OCH(CH₃)CH₂)_{y}—, and —(CH₂CH(CH₃)O)_{y}—,

a is an integer from 1 to 10;

each R^{N} is selected from H and C₁₋₃ alkyl;

each y is an integer from 1 to 2,000; and

Q is a residue of a quencher.

In some embodiments, R⁶ is H. In some embodiments, R⁶ is CH₃. In some embodiments, R⁶ is C₁₋₆ alkyl. In some embodiments, R⁶ does not comprise a carboxyl group and does not promote degradation of TCO in Formula (II) or Formula (B).

In some embodiments, a is an integer from 4 to 10. In some embodiments, a is an integer from 3 to 7. In some embodiments, a is at least 3. In some embodiments, a is 1, 2, 3, 4, 5, 6, or 7. In some embodiments, a is an integer from 1 to 7, and each L⁴ is independently selected from NH, C(═O), C₁₋₆ alkylene, C₆₋₁₀ arylene, and —(CH₂CH₂O)_{y}—. In some embodiments, each y is an integer from 1 to 10. In some embodiments, y is 1, 2, 3, 4, or 5.

In some embodiments, Y⁴ comprises a chemical group selected from an azide (—N₃), an aliphatic alkyne (—C≡CH), a cyclooctyne, a cyclooctene, a cyclohexene, a nitrone, an isocyanide, a cyclopropene, a norborene, a diphenylphosphine, nitrile imine, a tetrazole, a nitrile oxide, and a tetrazine.

In some embodiments, Y⁴ in formula (C) is complementary to Y³ in the conjugate of Formula (B) or Formula (II) as described herein, or a pharmaceutically acceptable salt thereof. In some embodiments, Y⁴ comprises an azide (—N₃) and Y³ comprises an aliphatic alkyne (—C≡CH) or a cyclooctyne. In some embodiments, Y⁴ comprises an aliphatic alkyne (—C≡CH) or a cyclooctyne and Y³ comprises an azide (—N₃). In some embodiments, Y⁴ comprises a cyclooctene or a cyclopropene and Y³ comprises a tetrazine. In some embodiments, Y⁴ comprises a tetrazine and Y³ comprises a cyclooctene or a cyclopropene.

In some embodiments, Y⁴ comprises a chemical group selected from any one of the following groups:

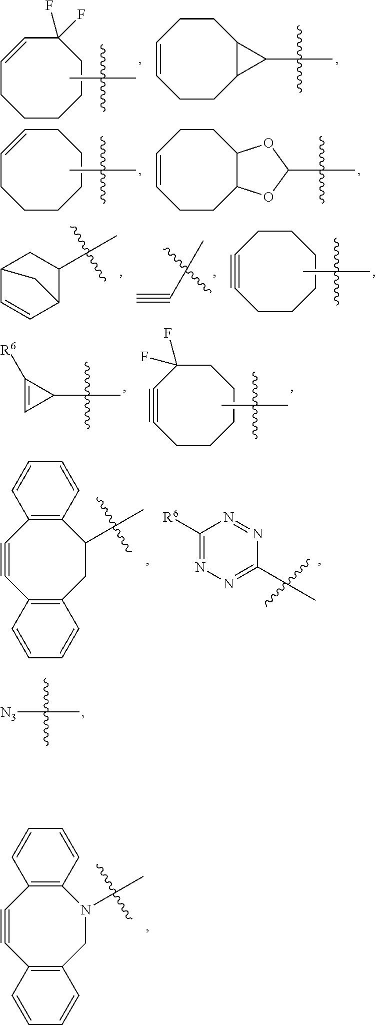

wherein R⁶ is selected from H and C₁₋₆ alkyl.

In some embodiments, Y⁴ comprises a chemical group selected from:

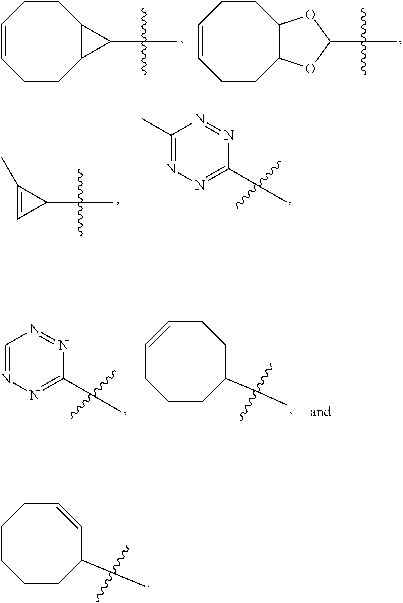

In some embodiments, compound of Formula (C) has formula:

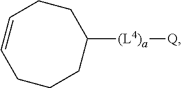

or a pharmaceutically acceptable salt thereof.

In some embodiments, the compound of Formula (C) has formula:

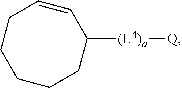

or a pharmaceutically acceptable salt thereof.

In some embodiments, the compound of Formula (C) has formula:

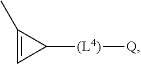

or a pharmaceutically acceptable salt thereof.

In some embodiments, the compound of Formula (C) has formula:

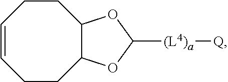

or a pharmaceutically acceptable salt thereof.

In some embodiments, the compound of Formula (III) has formula:

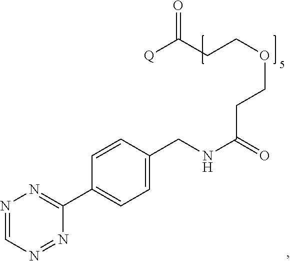

or a pharmaceutically acceptable salt thereof.

In some embodiments, the quencher Q is a fluorescence quencher. Suitable examples of fluorescence quenchers include aromatic azo compounds and phenazine derivatives. In some examples, the fluorescence quencher is BHQ0, BHQ1, BHQ2, BHQ3 (see, e.g., FIG. 1B), BHQ10, and IRDye QC-1.

In some embodiments, the contacting of step (iii) results in decrease of the fluorescence (or complete quenching of the fluorescence) of the fluorophore in the conjugate of Formula (II). Without being bound by a theory, it is believed that the quencher Q quenches the fluorescence of the fluorophore of Formula (II) through FRET quenching, that is, the excited fluorophore instead of emitting light transfers energy to the quencher through space (See FIG. 1C). In the absence of the quencher, the fluorophore would have emitted the light, which could have been detected. In some embodiments, the Q of Formula (III) and the fluorophore of Formula (II) are selected such that the emission spectrum of the fluorophore substantially overlaps with the absorption spectrum of the quencher Q.

Without being bound by a theory, in one example, it is believed that the TCO moiety in the protein conjugate of Formula (II) reacts with the tetrazine moiety of the Formula (III) to produce a protein conjugate of Formula (IV), as shown, for example, in Scheme 1.

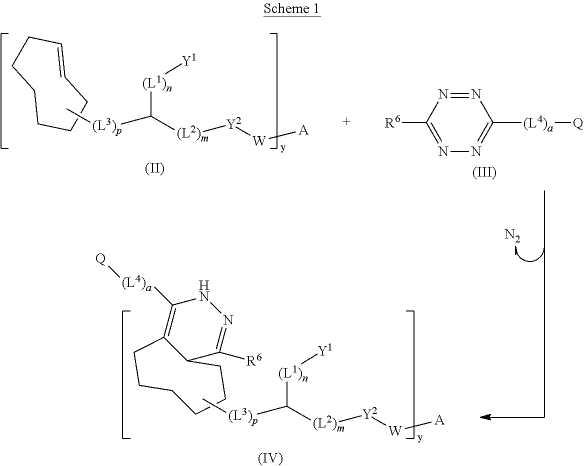

Referring to Scheme 1, the cyclooctene fragment of Formula (II) is in trans configuration. Hence, the trans-cyclooctene (TCO) and the tetrazine of Formula (III) engage in inverse-demand Diels Alder reaction followed by a retro-Diels Alder reaction to eliminate nitrogen gas. Through this ligation, the fluorophore of Y¹ and the quencher Q in the compound of Formula (IV) are covalently connected. Without being bound by a theory, it is believed that the spatial proximity between Q and the fluorophore of Y¹, created by covalent link between these groups, allows for efficient quenching of fluorescence.

In some embodiments, the present disclosure provides a tridentate linker of formula:

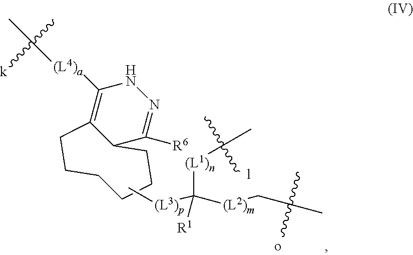

wherein 1 denotes a point of attachment to a fluorophore, O denotes a point to attachment to a protein, k denotes a point of attachment to fluorescence quencher, L¹, n, L², M, R¹, L³, and p are as described herein for Formula (II), and L⁴, a, and R⁶ aree as described herein for Formula (III).

### METHODS OF USE

In some embodiments, the present disclosure provides a method of profiling a cell, the method comprising (i) obtaining the cell from a subject, and (ii) examining the cell according to the methods of cellular analysis described herein.

In some embodiments, the present disclosure provides a method of examining a cell using a cytometry technique, the method comprising (i) obtaining the cell from a subject, and (ii) examining the cell according to the method of cellular analysis described herein. Suitable examples of cytometry techniques include image cytometry, holographic cytometry, Fourier ptychography cytometry, and fluorescence cytometry.

In some embodiments, the present disclosure provides a method of diagnosing a disease or condition of a subject by examining pathology of a cell obtained from the subject, the method comprising (i) obtaining the cell from a subject, and (ii) examining the cell according to the method of cellular analysis described herein.

In some embodiments, the present disclosure provides a method of monitoring progression of disease or condition (or monitoring efficacy of treatment of disease or condition) of a subject by examining pathology of a cell obtained from the subject, the method comprising (i) obtaining the cell from the subject, and (ii) examining the cell according to the method of cellular analysis described herein. The method allows to guide therapeutic regimens based on the results of examination of the cell according to the methods, and to provide individualized treatments.

In some embodiments, the present disclosure provides a method of monitoring efficacy of treatment of cancer. Suitable examples of cancer treatments include chemotherapy, radiation therapy, and surgery, or any combination of the foregoing.

Suitable examples of chemotherapeutic treatments include abarelix, aldesleukin, alitretinoin, allopurinol, altretamine, anastrozole, arsenic trioxide, asparaginase, azacitidine, bexarotene, bleomycin, bortezomib, busulfan, calusterone, capecitabine, carboplatin, carmustine, chlorambucil, cisplatin, cladribine, clofarabine, cyclophosphamide, cytarabine, dacarbazine, dactinomycin, dalteparin, dasatinib, daunorubicin, decitabine, denileukin, dexrazoxane, docetaxel, doxorubicin, dromostanolone propionate, emtansine, epirubicin, eribulin, erlotinib, estramustine, etoposide, everolimus, exemestane, fentanyl citrate, filgrastim, floxuridine, fludarabine, fluorouracil, fruquintinib, fulvestrant, gefitinib, gemcitabine, ozogamicin, goserelin acetate, histrelin acetate, tiuxetan, idarubicin, ifosfamide, imatinib, interferon α2a, irinotecan, ixabepilone, lapatinib, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, oxaliplatin, paclitaxel, pamidronate, panitumumab, pegaspargase, pegfilgrastim, pemetrexed, pentostatin, pipobroman, plicamycin, procarbazine, quinacrine, rasburicase, sorafenib, streptozocin, sulfatinib, sunitinib, sunitinib, tamoxifen, temozolomide, teniposide, testolactone, thalidomide, thioguanine, thiotepa, topotecan, toremifene, tretinoin, uracil mustard, valrubicin, vinblastine, vincristine, vinorelbine, volitinib, vorinostat, and zoledronate, or a pharmaceutically acceptable salt thereof.

In some embodiments, cancer treatment comprises administering to a patient an antibody useful in treating cancer. Suitable examples of such antibodies include pembrolizumab, nivolumab, cemiplimab, atezolizumab, avelumab, durvalumab, abagovomab, adecatumumab, afutuzumab, alacizumab pegol, altumomab pentetate, amatuximab, anatumomab mafenatox, apolizumab, arcitumomab, bavituximab, bectumomab, belimumab, bevacizumab, bivatuzumab mertansine, blinatumomab, brentuximab vedotin, cantuzumab mertansine, cantuzumab ravtansine, capromab pendetide, cetuximab, citatuzumab bogatox, cixutumumab, clivatuzumab tetraxetan, dacetuzumab, demcizumab, detumomab, drozitumab, ecromeximab, eculizumab, elotuzumab, ensituximab, epratuzumab, etaracizumab, farletuzumab, figitumumab, flanvotumab, galiximab, gemtuzumab ozogamicin, girentuximab, ibritumomab tiuxetan, imgatuzumab, ipilimumab, labetuzumab, lexatumumab, lorvotuzumab mertansine, nimotuzumab, ofatumumab, oregovomab, panitumumab, pemtumomab, pertuzumab, tacatuzumab tetraxetan, tositumomab, trastuzumab, totumumab, rituximab, alemtuzumab, durvalumab, ofatumumab, elotuzumab, and zalutumumab.

Suitable examples of cancer treatments also include immunotherapy. In some embodiments, the cancer treatment comprises a checkpoint inhibitor. In some embodiments, the checkpoint inhibitor is selected from anti-PD-1, anti-PD-L1, anti-CTLA-4, anti-CD20, anti-SLAMF7, and anti-CD52 (e.g., any one of the anticancer antibodies described above).

In some embodiments, the present disclosure provides a method of detecting a disease biomarker in a cell, the method comprising (i) obtaining the cell from a subject, and (ii) examining the cell according to the method of cellular analysis described herein.

In some embodiments, the cell is obtained from the subject using image-guided biopsy, fine needle aspiration (FNA), surgical tissue harvesting, punch biopsy, liquid biopsy, brushing, swab, touch-prep, fluid aspiration or blood analysis. In some embodiments, the cell is obtained from the subject using fine needle aspiration (FNA). In some embodiments, the cell is obtained from a tissue sample, such as a paraffin embedded (FFPE) tissue sample, a fresh tissue sample, or a frozen tissue sample. In some embodiments, the cell is selected from a cancer cell, an immune system cell, and a host cell (the methods of the present disclosure are useful for hepatocyte profiling in liver disease etc.). In some embodiments, the cell is a cancer cell. In some embodiments, the cancer cell is infected with human papillomavirus (HPV). In some embodiments, the cancer is caused by human papillomavirus (HPV). In some embodiments, a cellular sample obtained from the subject or from a tissue of the subject is scant or abundant. In some embodiments, the methods and reagents of the present disclosure are suitable for cellular samples and tissue samples containing any quantity of cells.

In some embodiments, the disease or condition (which can be diagnosed, monitored, or biomarker of which can be detected using the present methods) is cancer. In some embodiments, the methods disclosed herein allow to determine the composition of the tumor microenvironment. Suitable examples of cancer include lymphoma, breast cancer, skin cancer, head and neck cancer, head and neck squamous cell carcinoma (HNSCC), and oral cancer. Other examples of cancers include colorectal cancer, gastric (gastrointestinal) cancer, leukemia, melanoma, and pancreatic cancer, hepatocellular carcinoma, ovarian cancer, endometrial cancer, fallopian tube cancer, lung cancer, medullary thyroid carcinoma, mesothelioma, sex cord-gonadal stromal tumor, adrenocortical carcinoma, synovial sarcoma, bladder cancer, smooth muscle sarcoma, skeletal muscle sarcoma, endometrial stromal sarcoma, glioma (astrocytoma, ependymoma), rhabdomyosarcoma, small, round, blue cell tumor, neuroendocrine tumor, small-cell carcinoma of the lung, thyroid cancer, esophageal cancer, and stomach cancer. The technology is useful for any cancer detectable and compatible with biopsy by direct visualization, palpation, or image guidance.

In some embodiments, the cell is an immune cell. In some embodiments, the cell is selected from a hematopoietic cell, a T cell, a B cell, a NK cell, a myeloid cell, a macrophage, a dendritic cell, a neutrophil, and a monocyte.

Application of these methods is described more fully as follows.

The linkers, reagents, compounds, and methods of the present disclosure can be used at a point-of-care settings. In developed countries, repeat biopsies of ever-smaller lesions are straining accuracy and throughput, while low- and middle-income countries face extremely limited pathology and imaging resources, large case loads, convoluted and inefficient workflows, and lack of specialists. Advantageously, the compounds and methods described here allow for highly precise analysis of scant cancer samples, particularly those obtained by fine needle aspiration of mass lesions.

Accordingly, in some embodiments, the present disclosure provides an image cytometer that allows for automated cell phenotyping of scant cell samples. Various device applications for the methods and compounds of the present applications are described below.

Cellular cancer diagnostics are essential to clinical decision making: establishing the correct diagnosis, choosing the appropriate treatment, enrolling patients in experimental trials, assessing therapeutic efficacy and/or re-staging disease. Today, cancer specimens are commonly obtained by image-guided biopsy, fine needle aspiration (FNA), surgical tissue harvesting, punch biopsies, brushings, swabs, touch-preps, fluid aspiration or blood analysis (leukemia, lymphoma, liquid biopsies). Some of these methods (core and open surgical biopsies for histopathology) yield abundant tissue for sectioning and staining while others (FNA, brushings, touch-preps for cytopathology) yield scant cellular materials. FNA can often be obtained with minimal intervention using small-gauge needles (20-25 G), have very low complication rates and are generally well tolerated.

Rapid onsite assessment of cellular specimens has become increasingly important to narrowing the time between intervention and initiation of therapy, assuring specimen quality for subsequent diagnoses and minimizing sample degradation and loss during transport. The current workflows are still labor intensive and often centralized, requiring extensive sample processing and expert cytopathology review. Digital cytopathology and whole-slide imaging have been implemented but also require significant time, labor and investment. Taken together, these factors limit throughput, cost and global reach. A particular challenge is reliably analyzing scant cells either via manual imaging (requiring a trained cytopathologist reviewing an entire slide) or automated analysis (incorporating machine learning routines for automated diagnoses).

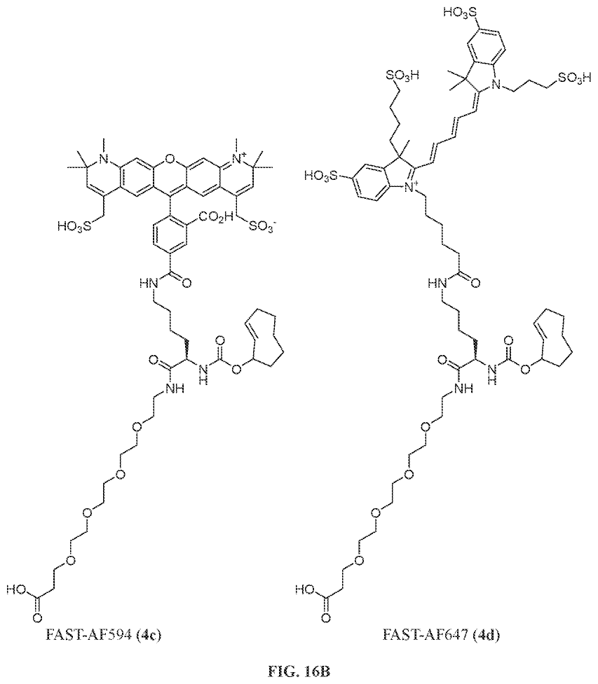

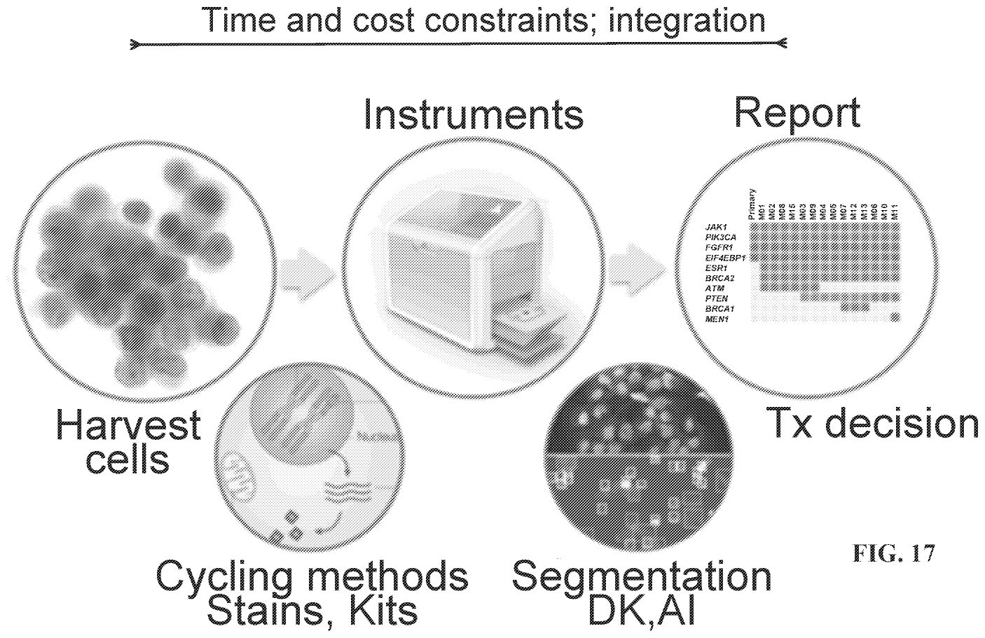

The present compounds and methods can be used in automated molecular image cytometers that use advanced materials, engineering and artificial intelligence (AI) for digital cell phenotyping. These new “all-in-one” systems address a potentially large clinical need by enabling advanced cellular diagnostics well suited to: 1) a global health market that is currently underserved; 2) repeat sampling at ultra-low morbidity since smaller needles are used (important for repeat sampling in clinical trials); 3) faster turn-around times (time saved by point-of-care analysis and neither embedding nor staining cores); 4) better and automated quality control and 5) invoking automation to reduce both time to diagnosis and the variability of interpretation. In addition, the present compounds and methods can be used in low-cost flow cytometers, liquid biopsies focusing on cfDNA, exosomes, circulating tumor cells (CTCs), and genomic screening tools (F1CDx, MSK-IMPACT). In some embodiments, the present compounds and methods are useful in automated analysis of cellular specimens obtained by tumor FNA (FIG. 17). In some embodiments, the present disclosure provides, in addition to the miniaturized and automated cytometry systems for desktop, point-of-care application described here, a high-throughput device useful for analysis of samples in centralized laboratories, such as CLIA labs.

Generic Cellular Stains

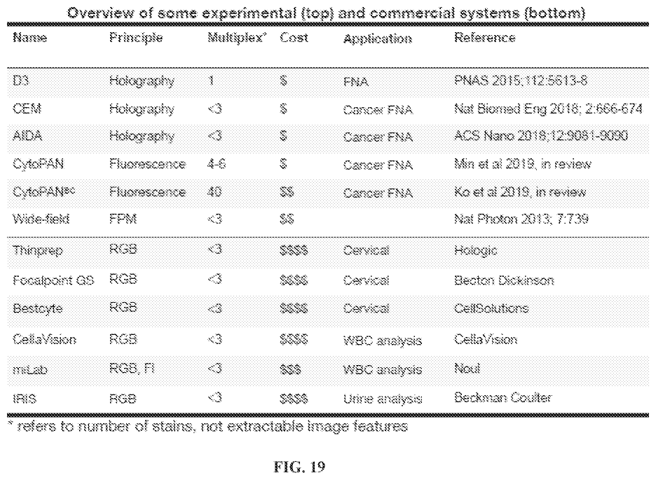

Conventional cytopathology largely relies on chromogenic stains such as hematoxylin and eosin (H&E), Papanicolaou (PAP) and Giemsa. Stained specimens are reviewed by cytopatholgists who evaluate cells for a number of parameters, for example, nuclear/cytoplasmic ratio, nuclear features, mitoses, clusters, cell uniformity, and cohesiveness. Such analyses can be automated but are inherently limited, resulting in variable diagnostic accuracies and lack of molecular information. Most commercial cell analyzers (FIG. 19) use this approach for automated white blood cell (WBC) analysis rather than cancer detection. Alternative dyes to investigate nuclear morphology (aneuploidy, segmentation) include DAPI, acridine orange, ethidium iodide, propidium iodide or flavins. Given the limitations of generic chromogenic staining, immunostaining for cancer-associated and host cell markers has emerged as an alternative and is being used widely in CTC analysis.

Antibody Staining

Antibodies are increasingly used in cytopathology and the standard is to perform one stain at a time, primarily using immunocytology (absorption measurements of antibody-enzyme-mediated chemical reactions) rather than immunofluorescence (emission measurements of fluorescently labeled antibodies). The compounds and methods of the present disclosure allow to detect a key molecular biomarker (e.g., cancer biomarker) while allowing morphological assessment of cells (e.g., cancer cells), for example, HER2 immunostaining in H&E slides.

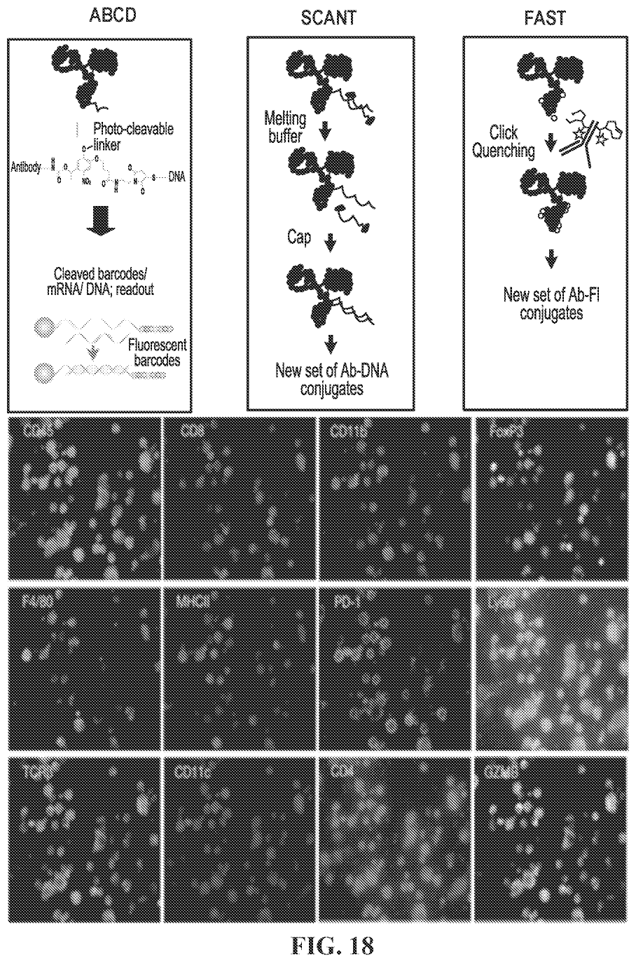

Multichannel fluorescence imaging (typically 4-6 channels) can be used to obtain more stains on a given cell, similar to flow cytometry, albeit at the cost of detailed cellular morphological information. To further improve the number of channels and markers (>20), cycling technologies have been developed that can repeatedly stain, destain and re-stain cancer tissues, ultimately allowing the number of markers per cell to be increased. This in turn facilitates deeper cell-by-cell profiling, pathway analysis and immunoprofiling in scant FNA. Most cycling methods were originally developed for paraffin-embedded tissue sections that can withstand harsh destaining conditions. Unfortunately, these harsh conditions, requiring oxidants for bleaching, are often incompatible with FNA samples. Furthermore, it was not uncommon for early cycling technologies to require days to process samples. Several different cell-compatible cycling technologies have been developed in recent years (FIG. 18). The more recent SCANT (single cell analysis for tumorphenotyping) method (FIG. 18) was shown to be robust and useful for pathway analysis in a clinical setting. One of the obstacles with SCANT, however, was its comparatively low SNR and relatively long destaining times (0.5-1 hour), similar to other cycling techniques. The methods and compounds of the present disclosure (e.g., FAST method) bypasses these shortcomings and allows extremely fast cycling (>95% quenching in <10 sec; FIG. 18).

Choice of Biomarkers

Selecting appropriate molecular markers is essential to identifying cells (e.g., cancer cells), differentiating them from host cells and profiling a growing number of treatment-relevant immune cells. While host cell markers have been thoroughly characterized by extensive flow cytometry studies, epithelial cancer markers are more diverse and thus require more stains. Furthermore, tumor markers are typically only expressed in a fraction of cancer cells and cases. The compounds and methods of the present disclosure allow to stain the following combinations of biomarkers: i) EpCAM, cytokeratins (CK), CD45 and CD16; ii) multi-marker combinations comprising for example EGFR, EpCAM, MUC1 and WNT2 (“Quad” marker”); iii) HER2, ER/PR for breast cancer; iv) CD19/20, k, l, Ki67 for lymphoma; v) EGFR, TTF1, chromogranin, synaptophysin for lung cancer; vi) EpCAM, calretinin, CD45, vimentin (ATCdx) for ovarian cancer and markers for mutated proteins such as KRASG12d, EGFRv3, IDH1132Gand BRAFV600E, among others.

Optimizing Materials for Cellular Analysis

Freshly obtained clinical samples have to be fixed, stained and captured on glass before they can be analyzed. All of these steps require careful optimization and often modified materials. Fixation can usually be done in paraformaldehyde, methanol/propanol or other commercially available mixes such as CytoRich Red (CRR). We have found empirically that some samples are better preserved in 50% diluted CRR, while fixation length (ideally 15-30 minutes) is less critical.

Immunostaining is best performed in small plastic vials by adding antibody reagents to cells in a staining buffer. Antibody-fluorochrome stability, quality control issues and limited access to basic tools (centrifuge, filters) are notable hurdles when using immunostains in remote areas and in point-of-care (POC) devices. Use of lyophilized antibodies and “cocktails” that contain all necessary ingredients can reduce variability. An alternative is to stain cells directly on glass slides after capture. Capturing cells on a glass slide is also critical to ensure that cells can be brought to the focal plane. Capture can be done using biological “glues” such as dopamine, biotin/neutravidin or polylysine as slide coatings. Alternatively, glass slides can be coated with capture antibodies. Irrespective of the method used, careful validation is required for different applications. Non-specific binding is typically reduced by coating slides with blocking materials such as BSA or PEG polymers. In order to simplify sample handling and processing, commercial systems may adapt cartridges to perform all of the above steps in a single platform.

Image Cytometry Systems

To inspect heterogeneous cell populations with statistical confidence, image cytometers must visualize large numbers of individual cells. Conventional geometric optics, however, are inherently constrained by the so-called space-bandwidth product (SBP and therefore produce megapixel information. This translates to a familiar experience: common microscopes have either wide field-of-view (FOV) at low resolution or small FOV at high spatial resolution but not both at the same time.

Most laboratory imaging systems overcome this limit by combining high-magnification optics with scanning stages to automatically scan slides and then transmit the information. The technologies of whole side imaging (WSI) and digital cytopathology have progressed over the years but challenges remain. Two key issues in digital cytopathology are i) focusing and ii) the remaining need for expert review. The focusing issue has largely been solved via either autofocusing hardware/software or 3D imaging of thick z-stacks. Autofocusing software often uses either a least squared or a mean value method to locate the ideal focus plane. 3D imaging, such as microscopy with optical sectioning, requires confocal laser scanning microscopy (CLSM), two-photon (2P) microscopy, structured illumination microscopy (SIM), light sheet fluorescence microscopy (LSFM) or Inverted selective plane illumination microscopy (iSPIM). All of these methods entail expensive instrumentation, require expert users and often generate/produce very large data sets. As such, this particular approach limits deployment in resource-constrained remote locations.

New technological advances increasingly enable automated molecular image cytometry, which is particularly helpful for POC use. Computational optics, wherein optically encoded images are digitally interpreted, can expand the SBP beyond optics' physical limit. Advances in optoelectronics and micro-optics further enable the construction of compact, easy-to-control, yet high-performance systems. Using these approaches can also decrease the overall system cost, as optoelectronical parts and computation have become inexpensive. Here we highlight three emerging modalities embodying these new concepts: digital holography, Fourier ptychography and miniaturized fluorescence cytometry.

Miniaturized Fluorescence Cytometry

As the list of known tumor markers grows, the need for multiplexed cellular profiling also increases, largely driven by interest in improving diagnostic accuracy, allowing patient triaging and facilitating molecularly based treatment decisions. Conventional immunocytology, which is based on chromogenic staining and brightfield microscopy, typically probes only for a few markers simultaneously. Fluorescent imaging, particularly in combination with cycling technologies, is a potent approach to in-depth multiplexing; a major technical challenge is to transform bulky, expensive microscopes into compact, affordable equivalents for POC uses. Fortunately, recent advances in optoelectronics have made available high-quality mini optical 8 parts, prompting new systems engineering. For example, small LEDs can deliver sufficient power to replace conventional lamps or lasers as an excitation light source, and the photosensitivity of semiconductor imagers has improved significantly for reliable low-light detection. Another opportunity is to augment manual image curation with automated analyses using machine learning approaches.

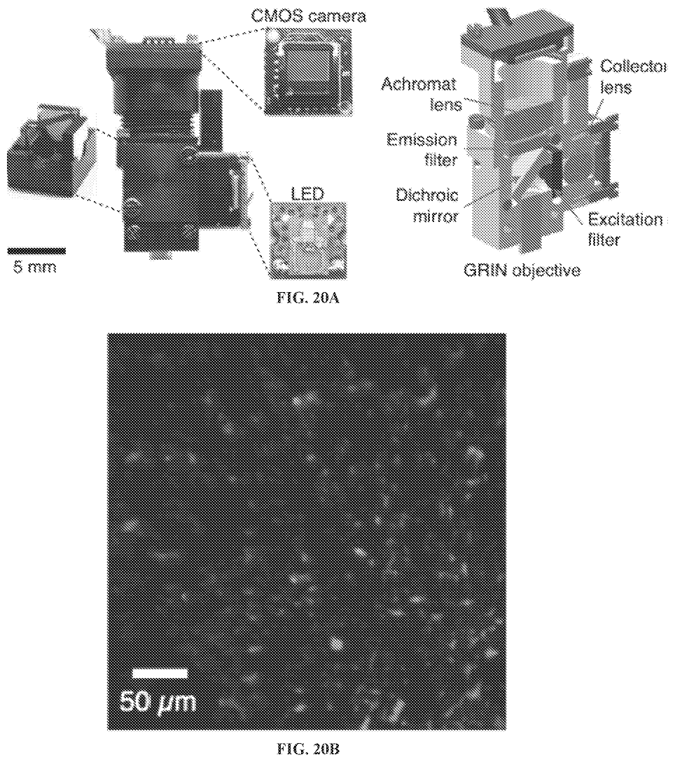

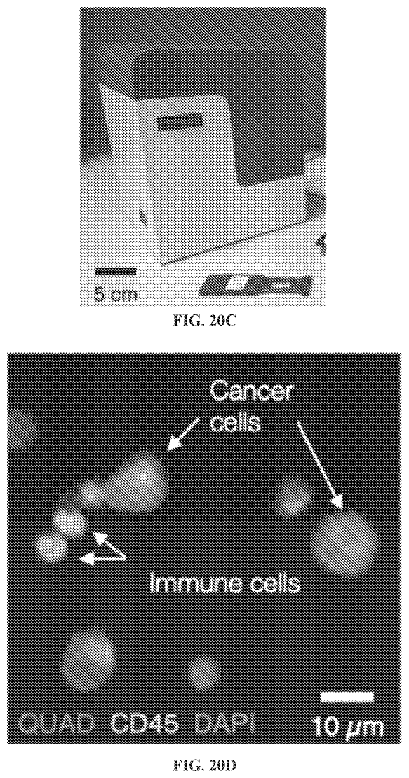

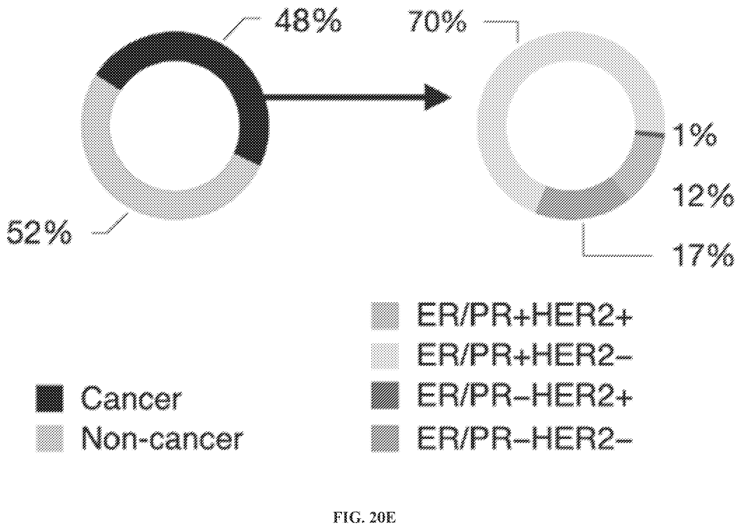

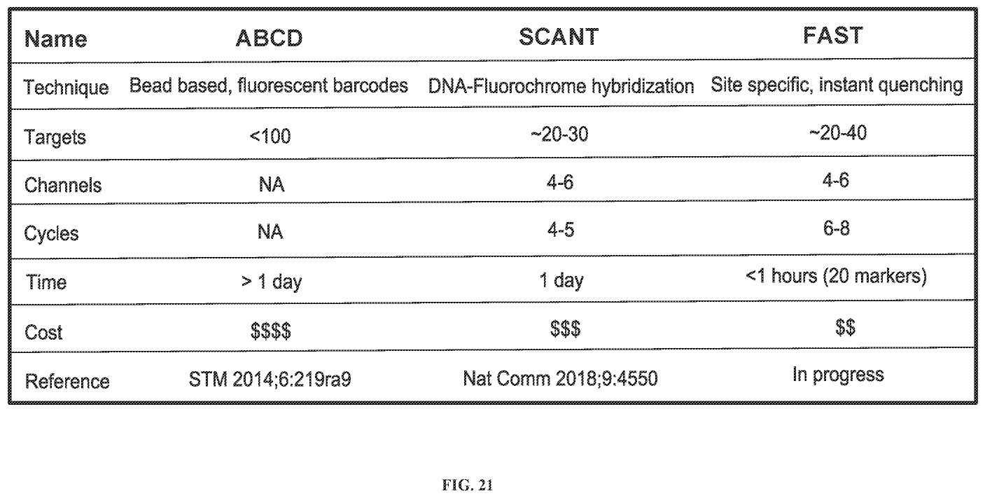

Thumb-sized fluorescent microscopes (“miniscopes”) integrate optical components into a single device (FIG. 20A). Using a gradient refractive index (GRIN) objective lens makes possible to shorten the optical path and drastically reduce system size (2.4 cm³, 1.9 g). Such a small form factor allowed the scope mountable on an animal's head with minimal interruption to its natural behavior and to image live neuron cells. As potential POC applications, miniscopes have been used for cell profiling and bacterial detection (FIG. 20B). In addition, a miniscope array performed large-area imaging without scanning, taking advantage of the scope's small lateral size (˜5 mm). System modification and computational processing enabled two-photon excitation, volumetric rendering or lens-less imaging.

For simultaneous multi-color (≥4) cellular analyses, Cytometry Portable Analyzer (CytoPAN) can be used. The system was originally built for operation in remote locations (FIG. 20C) but has additional applications in POC settings (OR, interventional suites, doctors' offices). The excitation light sources were positioned for side illumination through a glass slide, and a single emission filter with four pass bands was used. No dichroic mirrors or mechanical filter changes were necessary. Furthermore, intelligent software streamlined the entire assay, including light-source calibration, sample slide detection, data acquisition and cellular analyses. CytoPAN had four different fluorescent channels (FIG. 20D) and a bright-field imaging capacity. Automated algorithms profiled analyzed individual cells and produced summary reports for cancer diagnosis (FIG. 20E). This affordable system (<$1,000), in which the compounds and methods of the present application are implemented, is operable by non-skilled workers.

The fluorescent systems discussed above are still bound by the physical SBP limit and there thus remains a trade-off between FOV and spatial resolution. Computational methods used in coherent imaging cannot be applied, because fluorescent emission does not carry phase information. A straightforward workaround is to combine sample scanning with miniaturized optics; a key technical requirement is to automate such operations including stage movement and imaging stitching. Equally important is the development of tools for reliable sample preparation, for example by connecting fluidic cartridges with cost-effective pumping systems. This would speed up assays and minimize procedural errors particularly in cyclic imaging, which requires repeated fluidic handling such as quenching, washing, and labeling.

Conclusion

In contemporary laboratory medicine, virtually all blood and urine tests have been automated to reduce cost, improve test quality and accommodate the increasing volume of clinical samples. The methods disclosed here allow for automation to be applicable for FNA analysis of cancer samples, particularly in resource-limited environments. Suitable example includes automated POC cytometry, including the rigorous evaluation of cellular markers, staining techniques and kit developments. Automated, AI-based diagnostic DNA-karyometry is another suitable application. Also automated image cytometry, molecularly testing cytology samples, and fluorescent in situ hybridization (FISH) for EGFR, KRAS and BRAF mutation and other cytogenetic abnormalities should be feasible with appropriate amplification strategies. Finally, the compounds and methods of the present disclosure provide the techniques for analyzing FNA specimens for disease (e.g., cancer) diagnosis and monitoring. Inexpensive automated cellular analyses and molecular testing may be contemplated for organ FNA obtained from liver, kidney or blood/bone marrow.

Preparing Protein-Reactive Reagents

In some embodiments, the present disclosure provides a method of preparing an activated ester of a compound comprising a carboxylic acid group, the method comprising

i) reacting the compound comprising a carboxylic acid group with an excess amount of an activating reagent to obtain a reaction mixture comprising the activated ester; and

ii) contacting the reaction mixture with a compound of Formula (D):

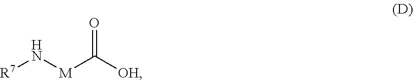

or a pharmaceutically acceptable salt thereof, wherein:

R⁷ is C₁₋₃ alkyl; and

M is C₂₋₆ alkylene;

wherein the contacting of the reaction mixture obtained in step i) with the compound of Formula (I), or a pharmaceutically acceptable salt thereof, deactivates the excess of the activating reagent in the reaction mixture.

In some embodiments, the activated ester is selected from N-hydroxysuccinimide (NHS) ester, nitrophenol ester, pentafluorophenol ester, and hydroxybenzotriazole ester.

In some embodiments, the compound comprising a carboxylic acid group is the compound of Formula (A) or Formula (I), or a pharmaceutically acceptable salt thereof, in which Y² is C(═O)OH.

In some embodiments, the activating reagent is selected from BOP, PyBOP, PyAOP, PyBrOP, BOP-Cl, HATU, HBTU, HCTU, TATU, TBTU, TDBTU, TSTU, TNTU, TPTU, DEPBT, and CDI, or a salt thereof.

In some embodiments, the activating reagent is TSTU:

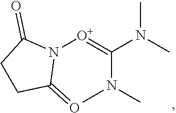

or a salt thereof (e.g., a pharmaceutically acceptable salt).

In some embodiments, the compound of Formula (D) is a compound ENBA of formula:

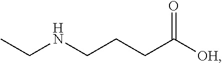

or a pharmaceutically acceptable salt thereof.

In some embodiments, the compound of Formula (D), or a pharmaceutically acceptable salt thereof, deactivates the excess of the activating agent by chemically reacting with the activating reagent.

In some embodiments, the chemical reaction between the compound of Formula (D) and the activating reagent produces a compound of Formula (E):

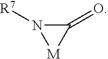

In some embodiments, the compound of formula (E) is:

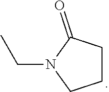

In some embodiments:

the activated ester is selected from N-hydroxysuccinimide (NHS) ester, nitrophenol ester, pentafluorophenol ester, and hydroxybenzotriazole ester;

the activating reagent is selected from BOP, PyBOP, PyAOP, PyBrOP, BOP-Cl, HATU, HBTU, HCTU, TATU, TBTU, TDBTU, TSTU, TNTU, TPTU, DEPBT, and CDI, or a salt thereof, and

the compound of Formula (D), or a pharmaceutically acceptable salt thereof, deactivates the excess of the activating reagent by chemically reacting with the activating reagent and forming a compound of Formula (E):

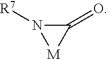

In some embodiments:

the activated ester is selected from N-hydroxysuccinimide (NHS) ester;

the activating reagent is TSTU:

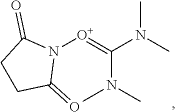

or a salt thereof,

the compound of Formula (D) is a compound ENBA of formula:

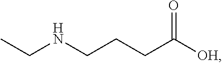

or a pharmaceutically acceptable salt thereof; and

the compound of Formula (D), or a pharmaceutically acceptable salt thereof, deactivates the excess of the activating agent by chemically reacting with the activating reagent and forming a compound of formula:

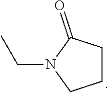

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “carboxy” refers to a —C(O)OH group.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl. The term “arylene” refers to a divalent aryl group, such as a phenylene.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃-10). In some embodiments, the cycloalkyl is a C₃₋₁₀ monocyclic or bicyclic cycloalkyl. In some embodiments, the cycloalkyl is a C₃₋₇ monocyclic cycloalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl. The term “cycloalkylene” refers to a divalent cycloalkyl group, such as cyclopropylene.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

### EXAMPLES

General Methods

Unless otherwise noted, reactions were carried out under an atmosphere of nitrogen or argon in air-dried glassware with magnetic stirring. Air- and/or moisture-sensitive liquids were transferred via syringe. All reagents were obtained from commercial sources at the highest grade available and used without further purification. Fluorophores were purchased from ClickChemistryTools or Fluoroprobes. BHQ®-3 Amine was purchased from LGC Biosearch Technologies. N-α-Boc-N-ε-Fmoc-Lysine was purchased from Chem-Impex. Amino-dPEG®₄-CO₂H was obtained from Quanta BioDesign. Dry solvents and coupling reagents were obtained from Sigma Aldrich. rTCO-PNP was a generous gift of Dr. Hannes Mikula (TU Wien, Austria). Anhydrous DMSO and DIPEA for microscale reactions were stored over 3 Å molecular sieves. Column chromatography was carried out with Biotage Snap C18 Bio flash cartridges and a Biotage Isolera Four instrument.

NMR spectra were recorded on a Bruker Avance UltraShield 400 MHz spectrometer. Chemical shifts are reported in parts per million (6) and calibrated using residual undeuterated solvent. Data are represented as follows: Chemical shift, multiplicity (s=singlet, d=doublet, t=triplet, q=quartet, p=pentet, m=multiplet, b=broad), coupling constant (J, Hz) and integration. High performance liquid chromatography-mass spectrometry analysis (HPLC-MS, LCMS) was performed with on a Waters instrument equipped with a Waters 2424 ELS Detector, Waters 2998 UVV is Diode array Detector, Waters 2475 Multi-wavelength Fluorescence Detector, and a Waters 3100 Mass Detector. Separations employed an HPLC-grade water/acetonitrile solvent gradient with one of two columns: XTerra MS C18 Column, 125 Å, 5 μm, 4.6 mm×50 mm column; Waters XBridge BEH C18 Column, 130 Å, 3.5 μm, 4.6 mm×50 mm. Routine analysis were conducted with 0.1% formic acid added to both solvents; buffered solvents were used when relevant, including for all separations of Alexa Fluor dye containing compounds, which exhibit significant retention anomalies on standard Waters reversed phase columns. Buffered analyses were run with ammonium formate buffer (2.5 mM)/HPLC-grade acetonitrile at either pH 4.5 or pH 8.4, for base-sensitive (e.g., NHS-activated esters) and acid-sensitive compounds, respectively. Fluorescence measurements were conducted with a PTI QuantaMaster 400 fluorimeter (Photon Technologies Incorporated, NJ, USA) or TECAN Spark plate reader, and UV-VIS absorption spectra on a Horiba DualFL spectrophotometer (Horiba Instruments) or Nandrop Spectrophotometer (ThermoFisher). PBS-Bicarb buffer (pH 9) was prepared by addition of 40 mM sodium bicarbonate/carbonate buffer (MicroEssentials) to PBS, and the final concentration adjusted by addition of milli-Q water to match isotonic physiologic osmolality.

Biological and Imaging Methods

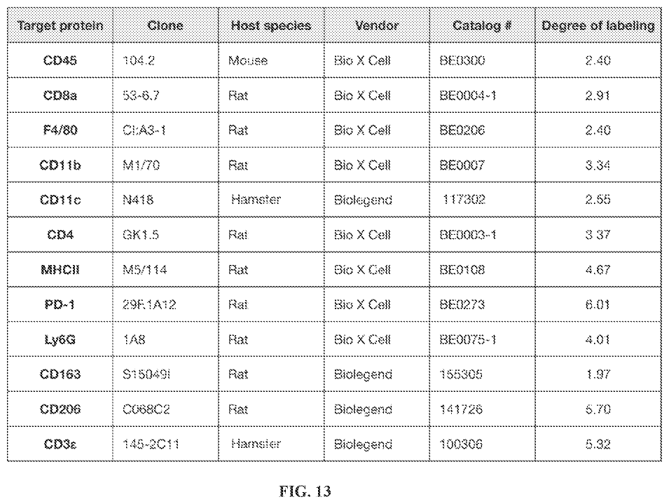

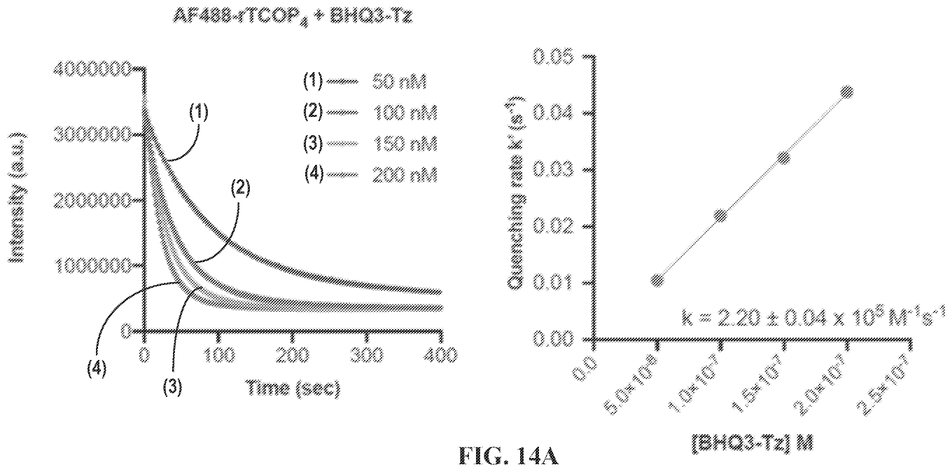

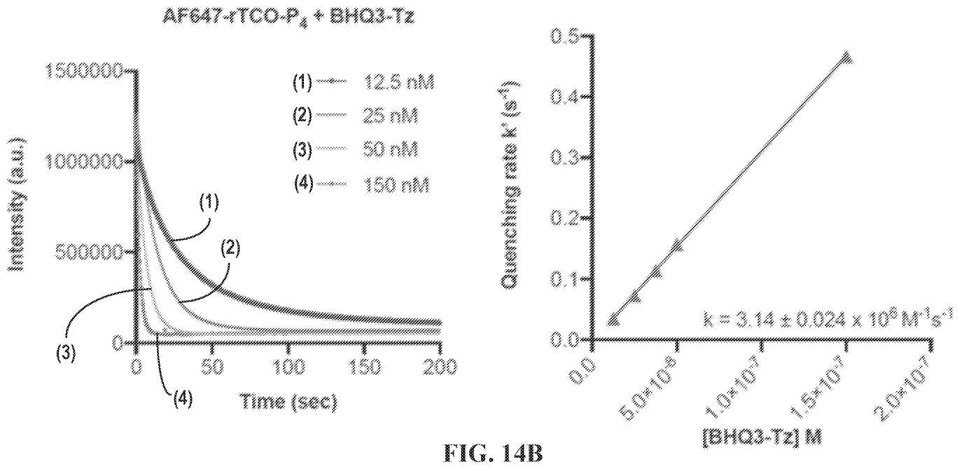

Antibodies. Cetuximab (anti-EGFR antibody, Erbitux) was used to test and optimize staining and quenching methods. Antibodies used to profile infiltrating immune cells in mouse tumor samples are summarized in FIG. 13 above. All antibodies were tested on positive cell lines or mouse splenocytes for validation before usage.

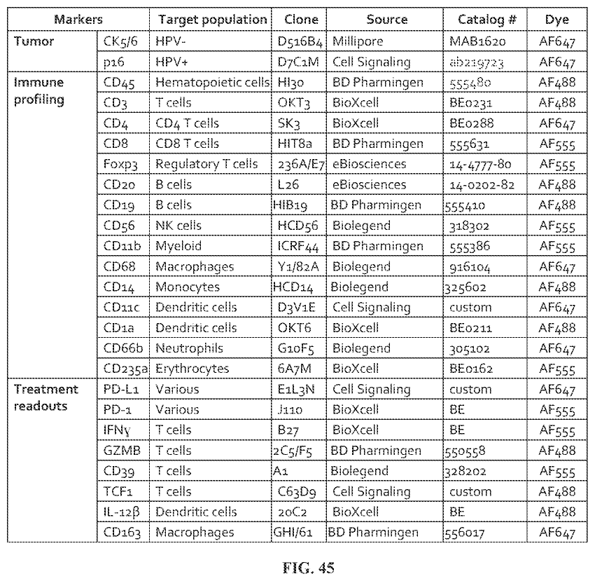

Vendors and catalog numbers of the antibodies used for immunoprofiling of mouse samples and clinical samples are summarized in FIGS. 44 and 45. All antibodies were tested and validated on positive cell lines, mouse splenocytes, or peripheral blood mononuclear cells (Innovative Research Inc.) for validation before usage. Anti-PD1 antibody for treatment of murine tumor models were purchased from Bio X Cell (Clone: 29F.1A12). The MC38 mouse colorectal cancer cell line was a kind gift from Mark Smyth, QIMR Berghofer Medical Research Institute), and MOC mouse oral cavity squamous cell cancer cell lines were purchased from Kerafast.

Antibody modification. BSA free antibodies were purchased as-is (FIGS. 44 and 45) and then modified with fluorophore-TCO conjugates (FAST probes) as described. Antibodies were exchanged into bicarbonate buffer (pH 8.4) using a 40k zeba column (Thermo Fisher). After buffer exchange, antibodies were incubated with a 5- to 12-fold molar excess of the activated Dye-TCO-NHS linker for 25 mins at room temperature. The conjugation reaction was loaded onto another 40k zeba column (equilibrated with PBS) for desalting and removal of unreacted dye molecules. The absorbance spectrum of the conjugated antibody was measured using a Nanodrop 1000 (Thermo Scientific) to determine the degree of labeling (DOL), applying the known extinction coefficients of the dye, IgG antibody, and correction factor (CF280) for the dye absorbance at 280 nm. The FAST-conjugated antibodies were stored in the dark at 4° C. in PBS until usage.

Cells. The A431 cell line was used to test and further optimize compounds. Cells were purchased from the American Tissue Culture Collection (ATCC). A431 cells were passaged in DMEM (10% FBS, 1% penicillin/streptomycin) according to the specifications from ATCC. Cells were first grown in a 150 mm cell culture dish and then seeded on Millicell 8-well EZ slides (Millipore) for imaging. After 24-48 hours, confluency was assessed and cells were fixed with 4% paraformaldehyde in PBS (10 min) prior to EGFR imaging. Additional cells tested included murine MC38 colorectal cancer cells (kind gift from Mark Smyth, QIMR Berghofer Medical Research Institute) from C57BL6 mice, mouse splenocytes and human peripheral blood mononuclear cells (MGH Blood Bank).

Animals. Female and male WT C57BL6 mice with 8-12 weeks of age were purchased from Jackson Laboratory for tumor implantation of MC38, MOC2, and MOC22 tumors. All animals were housed under specific pathogen free conditions at the Massachusetts General Hospital. Experiments were approved by the MGH Institutional Animal Care and Use Committee (IACUC) and were performed in accordance with MGH IACUC regulations.

Mouse tumor fine needle aspirate (FNA). C57BL6 mice (Jackson Laboratory) were injected subcutaneously with 2×10⁶ of MC38 mouse colorectal cancer cells, 2×10⁶ of MOC22 cells, or 0.5×10⁶ of MOC2 cells in 50 μl of sterile PBS. Since MOC2 is a very aggressive tumor, fewer MOC2 cells were injected to match the tumor growth rates of MOC2 and MOC22. When tumors reach ˜40 mm³ of sizes after 1-2 weeks of tumor growth on the lower back, mice were anesthetized with 2% isofluorane inhalation for the FNA procedure. FNAs were obtained by inserting and withdrawing the 22G needle within tumor tissue, applying slight negative pressure. This step was repeated several times per location similarly as is done clinically. The collected samples were flushed out of syringe with RPMI (tumor digestion media (collagenase type I, type IV, DNase I in HBSS)) into 1.5-mL Eppendorf tubes and kept on ice. FNA samples were digested for 15 minutes at 37° C., washed with PBS, and fixed in 4% paraformaldehyde. Fixed cells were then attached to glass slides with a Cytospin centrifuge system (Thermo Scientific, 850 rpm at 5 min spin time) prior to imaging. Octospot 8-well strips (Thermo Scientific) was used to attach cells in a defined area on glass slides.

Immunostaining and quenching. Cells were fixed for 10 minutes with 4% PFA and permeabilized for 15 minutes with 0.1% Triton-X100 (using BD Cytofix/Cytoperm buffer (BD Bioscience)) before staining. Immunostaining for FAST imaging was performed as ordinary immunofluorescence. After blocking with 1% BSA-PBS (Odyssey buffer (LI-COR Biosciences)) for 30 minutes, FAST-conjugated antibodies were diluted into 1-5 μg/ml in Odyssey buffer and incubated with cells for 15-30 mins at room temperature in the dark. Stained cells were washed with PBS before imaging. After imaging, 10-50 μM BHQ3-Tz was used to wash cells (<10 sec) for quenching in PBS-bicarb (pH 9), followed by 3 thirty second washes to remove free BHQ3-Tz. The cells were imaged again in the same fields of view to record quenched signal. In some applications, before antibody staining of subsequent cycle, cells were briefly incubated in a solution of 10 μM dTCO-PEG₆:

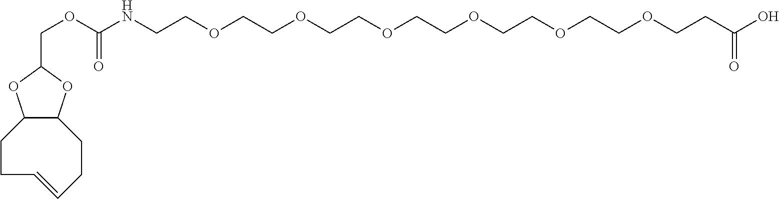 [SMILES: O=C(O)CCOCCOCCOCCOCCOCCOCCNC(=O)OCC1OC2CC/C=C/CCC2O1]

in order to block residual BHQ3-Tz from reacting with FAST antibodies of the next cycle. After quenching, the same staining, imaging, and quenching cycle was repeated for multiplexed protein profiling from the same cells.

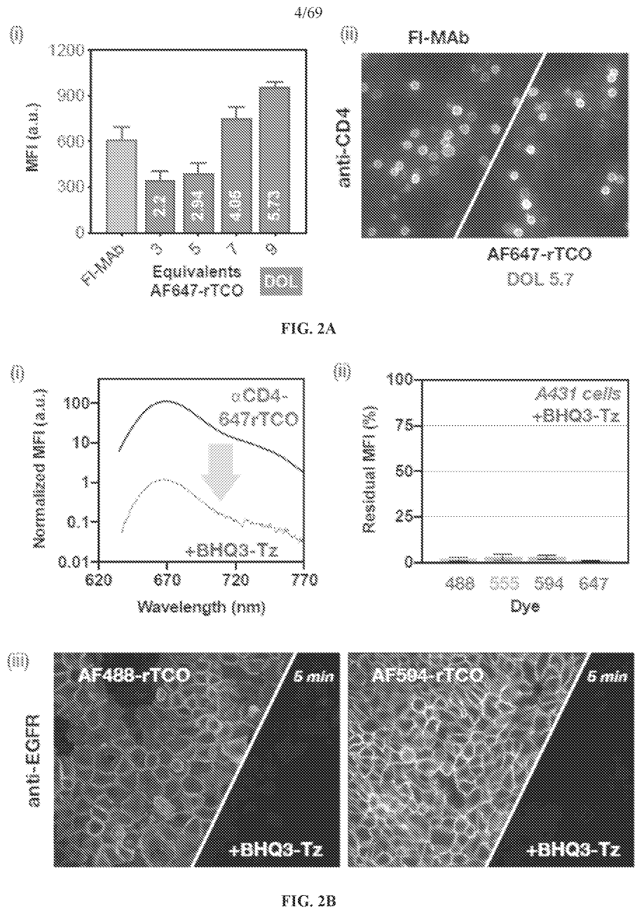

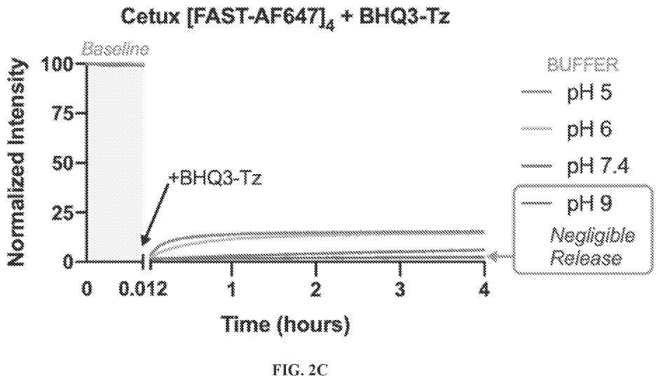

Fluorescent imaging and analysis. An Olympus BX-63 upright automated epifluorescence microscope was used to acquire fluorescent images. DAPI, FITC, Cy3, TRITC, and Cy5 filter cubes were used to excite DAPI/Hoechst nuclear stains, AF488, AF555, AF594, and AF647 fluorophores respectively. ImageJ and CellProfiler were used for cell segmentation and the measurement of fluorescent intensities in cells. Quenching efficiencies for fluorescent antibody imaging (FIG. 2B) were calculated from serial images: i) prior to staining (background); ii) after staining/washing (signal); iii) after BHQ3-Tz treatment (quench). Residual MFI=(quench−background)/(signal−background).

Flow Cytometry.

Mouse tumors and spleens were excised and minced using surgical scissors.

Tissues were then digested using collagenase type I (Worthington), collagenase type IV (Worthington), and DNase I (Worthington) in HBSS for 25 minutes at 37° C.

Digests were passed through a 70- m cell strainer (BD Falcon), and then washed with HBSS with 2% FBS. Samples were first incubated with True Stain FcX antibody (BioLegend) in PBS containing 0.5% BSA and 2 mM EDTA before staining with antibodies directly conjugated to fluorophores for flow cytometry. In addition to FAST labeled antibodies listed in Table 1, antibodies against CD3e (clone 145-2C11, Biolegend), CD4 (clone GK1.5, BD Biosciences), CD45 (clone 30-F11, Biolegend), CD8a (clone 53-6.7, Biolegend), CD11b (clone M1/70, Biolegend), CD11c (clone N418, Biolegend), F4/80 (clone BM8, Biolegend), MHC II (clone M5/114.15.2, Biolegend), CD25 (clone PC61, Biolegend), FoxP3 (clone MF-14, Biolegend), Ly6C (clone HK1.4, Biolegend), Ly6G (clone 1A8, Biolegend), and TCRb (clone H57-597, Biolegend) were used for validation of marker staining. Propidium iodide or 7-AAD (BioLegend) was used to exclude dead cells.

Human samples were processed similarly as described above. In addition to FAST labeled antibodies listed in Table 2, antibodies against CD3 (clone SK7, Biolegend), CD4 (clone SK3, Biolegend), CD45 (clone 2D1, Biolegend), CD8 (clone SK1, Biolegend), CD11b (clone ICRF44, Biolegend), CD11c (clone 3.9, Biolegend), CD14 (clone HCD14, Biolegend), CD19 (clone SJ25C1, Biolegend), CD56 (clone 5.1H11, Biolegend), CD66b (cloneG10F5, Biolegend), CD68 (clone Y182A, Biolegend), FoxP3 (clone 206D, Biolegend), p16 (clone D7C1M, Cell Signaling Technology), and PD-1 (clone EH12.2H7, Biolegend) were used for validation of marker staining. Zombie fixable viability kit (Biolegend) was used to label dead cells before fixing. After staining of cell surface markers, cells were fixed and permeabilized using Intracellular staining kit (Biolegend) to stain for intracellular markers. Cells were washed and filtered after staining and were then analyzed on a BD LSRII flow cytometer. AbC Total Antibody Compensation Bead Kit was used for single color compensation. Flow cytometry data were then analyzed using FlowJo software (Tree Star Inc.).

Clinical samples. The study was approved by the Institutional Review Board at Massachusetts General Hospital (IRB #2014P000559) and informed consent was obtained from all newly diagnosed and recurrent/metastatic HNSCC subjects. Three to 5 passes from minimally invasive fine needle aspirates were obtained from 15 patients. FNA samples that didn't yield a sufficient number of cells for immune profiling (less than 200 cells total) were excluded from the analysis.

Immunohistochemistry of surgical tissue sections. Tissue sections were stained for PD-L1 using the EL1N3 antibody [Udall et al., 2018, Diagn Pathol, 13, 12]. The composite positive score (CPS) was calculated as the number of PD-L1-positive cells (tumor cells, lymphocytes, and macrophages) divided by the number of viable tumor cells, and multiplied by 100. Although theoretically that quantity can exceed 100, the maximum score is defined as 100. A minimum of 100 viable tumor cells had to be present in the PD-L1-stained slide (sectioned tumor biopsy or resection tissue) for the specimen to be considered adequate for evaluation. Tumor cells had to show either partial or complete membrane staining to be counted as “stained,” whereas immune cells are counted if there is any staining.

### Example 1—Synthesis of FAST Probe Scaffold

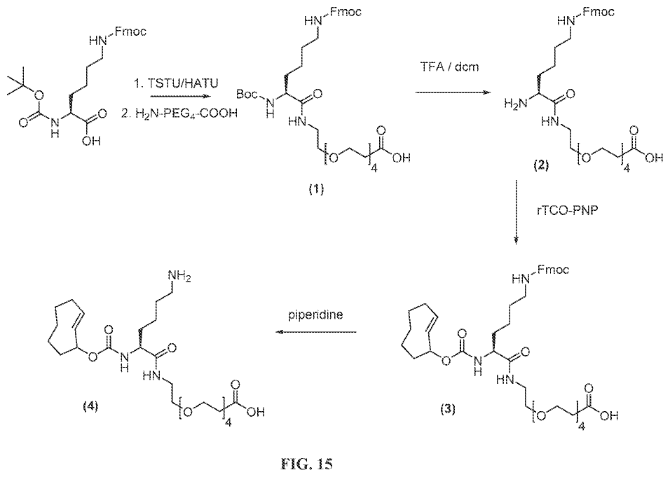

A scheme showing chemical synthesis of FAST probe (4) is shown in FIG. 15.

### Preparation of N-α-Boc-N-ε-Fmoc-Lysine-PEG₄-COOH (1)

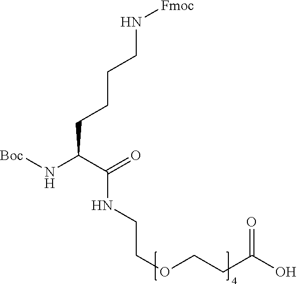

To a solution of N-α-Boc-N—F-Fmoc-Lysine (110 mg, 0.237 mmol, Chem-Impex) in anhydrous DMSO (850 μL) were added 1 equivalent (40.7 μL) of diisopropylethylamine (DIPEA) and 0.95 equivalents of TSTU (N,N,N′,N′-Tetramethyl-O—(N-succinimidyl) uronium tetrafluoroborate, 68 mg). The reaction was mixed by gentle rocking in an eppendorf tube at room temperature (rt) for five minutes, at which point LCMS indicated complete conversion to the NHS ester. The reaction mixture was then transferred by pipette to a second eppendorf tube containing 75 mg of H2N-PEG₄-COOH (1.2 equivalents) and an additional equivalent of DIPEA was added. After 30 minutes on a vigorous rotary mixer (1400 RPM) the PEG₄ amino acid had dissolved and LCMS indicated complete consumption of the NHS ester. The DMSO solution was loaded directly onto a 25 g Biotage SNAP Bio C18 column and the product (125 mg, 77%) obtained as a clear viscous oil after reversed phase chromatography (H₂O/MeCN gradient elution, 0.1% formic acid).

¹H-NMR (400 MHz; CD₂Cl₂): δ 7.81 (d, J=7.5 Hz, 2H), 7.65 (d, J=7.5 Hz, 2H), 7.44 (t, J=7.4 Hz, 2H), 7.35 (t, J=7.4 Hz, 2H), 4.47-4.42 (m, 2H), 4.25 (t, J=6.5 Hz, 1H), 4.14-4.12 (m, 1H), 3.76-3.73 (m, 2H), 3.60-3.55 (m, 14H), 3.46-3.42 (m, 2H), 3.20-3.15 (m, 2H), 2.60-2.59 (m, 2H), 1.80-1.77 (m, 1H), 1.67-1.61 (m, 1H), 1.53 (td, J=11.6, 6.1 Hz, 2H), 1.45 (d, J=15.2 Hz, 9H), 1.40-1.35 (m, 2H). ¹³C NMR (101 MHz, DMSO) δ 173.9, 172.4, 156.5, 155.8, 144.2, 141.3, 127.6, 127.0, 125.0, 119.9, 79.7, 70.5, 70.4, 70.2, 69.6, 66.56, 66.2, 54.3, 47.3, 40.5, 39.3, 35.0, 32.5, 29.3, 28.1, 22.5. MS [M-H]⁻ calcd. 714.36 for C37H52N3011, found 714.55.

### Preparation of N-ε-Fmoc-Lysine-PEG₄-COOH (2)

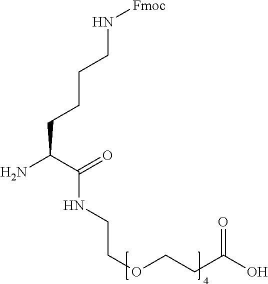

Trifluoroacetic acid (300 μL) was added to a solution of (1) (125 mg, 0.175 mmol) in 1.2 mL of DCM, for a final composition of 20% TFA/DCM. The mixture was allowed to stand in a capped vial at room temperature for 40 minutes and then rotovapped to yield a clear viscous oil. This material was resuspended in acetonitrile:toluene (1:1); serial rotary evaporation with acetonitrile/toluene was repeated until excess TFA had been substantially removed. The pale yellow oil was allowed to dry further on hi-vac overnight then dissolved in 1 mL of DMSO and loaded onto a 25 g Biotage SNAP Bio C18 column. Reversed phase purification (H₂O/MeCN gradient elution, 0.1% formic acid) gave the desired product as a clear oil (97 mg, 90.2%).

¹H-NMR (400 MHz; CD₂Cl₂): δ 8.62 (s, 1H), 8.39 (s, 1H), 7.82 (d, J=7.5 Hz, 2H), 7.67 (d, J=7.4 Hz, 2H), 7.44 (t, J=7.4 Hz, 2H), 7.36 (t, J=7.4 Hz, 2H), 5.85 (s, 1H), 4.42 (d, J=6.7 Hz, 2H), 4.25 (t, J=6.4 Hz, 1H), 4.07 (t, J=4.3 Hz, 1H), 3.72 (t, J=4.9 Hz, 2H), 3.59 (s, 14H), 3.47 (s, 2H), 3.18 (d, J=5.8 Hz, 2H), 2.54 (t, J=4.4 Hz, 2H), 1.88 (s, 2H), 1.55 (d, J=5.9 Hz, 2H), 1.46 (d, J=5.9 Hz, 2H). ¹³C NMR (101 MHz, CD₂Cl₂) δ 176.8, 169.9, 167.4, 156.9, 144.56, 141.6, 127.95, 125.4, 120.2, 70.52, 70.46, 70.35, 70.23, 70.21, 70.06, 69.94, 67.68, 66.5, 53.7, 47.7, 40.7, 39.7, 36.4, 31.5, 29.4, 22.2. MS [M-H]⁻ calcd. 614.32 for C₃₂H₄₄N₃O₉, found 614.44.

### Preparation of N-α-rTCO-N-ε-Fmoc-Lysine-PEG₄-COOH (3)

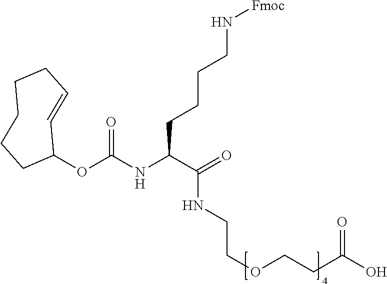

DIPEA (8.2 μL, 0.045 mmol, 1.05 eq.) was added to a solution of (2) (28 mg, 0.045 mmol) in DMSO (350 μL), followed by rTCO-PNP carbonate [See Ref 26] (11 mg, 0.8 eq., 0.036 mmol) and the mixture was gently rocked at rt for 20 min, at which point LCMS indicated ˜50% conversion. An additional aliquot of 6 μL of DIPEA was added to neutralize residual formic acid from the preceding column. LCMS analysis after an additional 20 minutes indicated complete consumption of rTCO-PNP. The reaction mixture was loaded directly onto a Biotage SNAP Bio C18 column that had been equilibrated in ammonium formate buffer (pH 9.1). The column was washed with five column volumes of 95:5 (pH 9.1 buffer: MeCN) to elute the p-nitrophenol, visually monitoring the disappearance of its intense yellow color from the column. The aqueous phase was then switched to 0.02% formic acid in water and the column was washed with an additional 4 column volumes of 95:5 (H₂O:MeCN, 0.02% formic acid). The product was then isolated with an acetonitrile gradient; after elution and rotary evaporation of the desired fraction, 21 mg of a white crystalline solid were obtained (75.2% yield).

¹H-NMR (400 MHz; CD₂Cl₂): δ 7.81 (d, J=7.5 Hz, 2H), 7.65 (d, J=7.5 Hz, 2H), 7.44 (t, J=7.4 Hz, 2H), 7.35 (t, J=7.4 Hz, 2H), 5.83 (m, 1H), 5.53 (d, J=16.6 Hz, 1H), 5.30 (s, 1H), 4.44 (s, 2H), 4.25 (t, 1H), 3.74 (t, 2H), 3.60 (m, 14H), 3.45 (m, 2H), 3.18 (m, 2H), 2.61-2.59 (m, 2H), 2.47 (d, J=10.0 Hz, 1H), 2.04 (m, 2H), 2.01-1.94 (m, 2H), 1.84 (m, J=13.4 Hz, 2H), 1.68 (m, J=13.4 Hz, 3H), 1.55-1.39 (m, 5H), 1.11 (m, 1H), 0.83 (m, 1H). ¹³C NMR (101 MHz; CD₂Cl₂): δ 173.8, 172.0, 156.5, 155.7, 144.2, 141.3, 131.8, 131.3, 127.6, 127.0, 125.0, 119.9, 74.3, 70.39, 70.33, 70.27, 70.11, 69.6, 66.6, 66.1, 54.5, 47.3, 40.60, 40.54, 39.2, 35.95, 35.1, 32.67, 32.58, 29.3, 29.0, 24.1, 22.3. MS [M-H]⁻ calcd. 766.39 for C₄₁H₅₆N₃O₁₁⁻, found 766.59.

### Preparation of N-α-rTCO-Lysine-PEG₄-COOH (4)

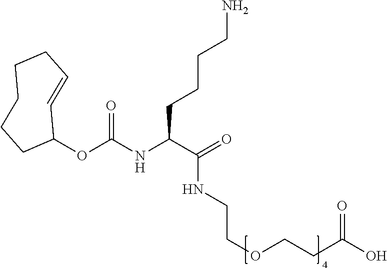

Piperidine (25 μL) was added to a solution of (3) (18 mg, 0.0234 mmol) in DMSO (300 μL) to give a final concentration of 7.5% (v/v). After 15 minutes the reaction mixture had solidified with a glassy matrix of deprotected Fmoc byproducts. 500 μL of hexanes were added to the vial, resulting in complete dissolution of the solid material and a biphasic mixture. The underlying DMSO layer was removed by pipette and loaded directly onto a Biotage SNAP Bio C18 column. Purification with a gradient of ammonium formate buffer (2.5 mM, pH 8.4)/MeCN gave the desired product as a clear oil (12.3 mg, 96%).

¹H NMR (400 MHz, CD₃OD): δ 5.89 (q, J=13.8 Hz, 1H), 5.57 (dd, J=16.4, 7.5 Hz, 1H), 5.26 (s, 1H), 4.11 (m, J=4.9 Hz, 1H), 3.74 (t, J=6.0 Hz, 2H), 3.64 (t, J=12.4 Hz, 14H), 3.51 (dd, J=14.1, 4.5 Hz, 1H), 2.95 (t, J=7.1 Hz, 2H), 2.47 (t, J=6.0 Hz, 3H), 2.07-1.98 (m, 3H), 1.93-1.71 (m, 7H), 1.51 (m, 3H), 1.19 (t, J=11.0 Hz, 1H), 0.90 (m, 1H). ¹³C NMR (101 MHz; CD₃OD, two rotamers): δ 177.9, 173.4, 156.4, 131.49, 131.42, 131.21, 131.17, 74.14, 74.07, 70.09, 70.03, 69.96, 69.88, 69.65, 69.53, 69.1, 67.9, 54.6, 40.21, 40.16, 39.1, 38.7, 37.4, 35.6, 35.42, 35.36, 31.34, 31.30, 28.66, 28.63, 26.52, 26.48, 23.87, 23.80, 22.2. MS [M-H]⁻ calcd. 544.33 for C₂₆H₄₇N₃O₉⁻, found 544.57.

### Example 2—Synthesis of BHQ3-Tz

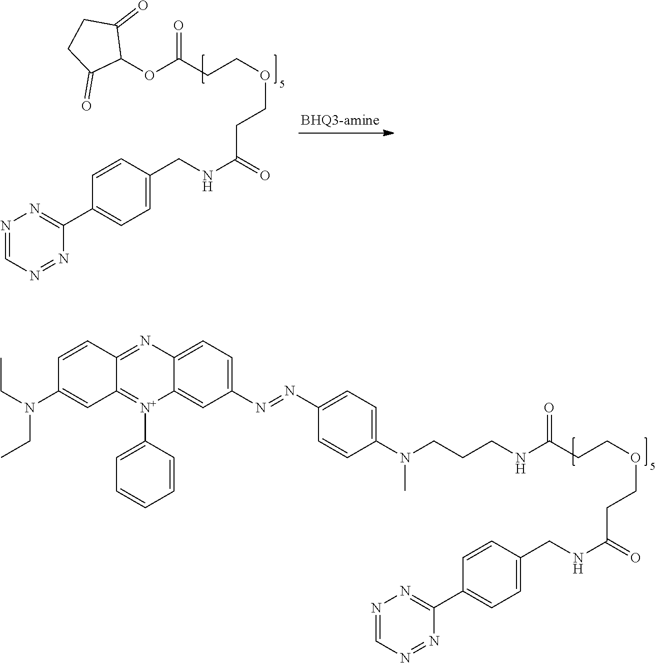

Synthesis of BHQ3-PEG₅-benzylamino-Tz-H. To a solution of benzylamino (H)-tetrazine-PEG5-NHS ester (5 mg, 8.27 μmol, ClickChemistry Tools) in 250 μL anhydrous DMSO was added 5 mg of BHQ3-Amine (1.16 equivalents, 9.63 μmol, Biosearch Technologies), followed by 1.56 μL of DIPEA (1.1 equivalents). The material was vortexed and sonicated to dissolve the BHQ3-amine and allowed to react for 5 minutes. Longer reaction times with H-tetrazines in this context can lead to significant degradation of the Tz. The reaction mixture was directly injected onto a Biotage Snap Bio C18 column and purified with a gradient elution of ammonium formate buffer (pH 4.5):acetonitrile. Substantial streaking and residual binding to the column were observed at lower buffer concentrations, requiring 5-10 mM to improve (but not fully remedy) elution characteristics. After rotary evaporation, the product was obtained as an intensely colored dark blue-green solid (3.4 mg, 41% yield, purity 94.6% by LCMS). MS [M]⁺ calcd. 1007.53 for C₅₅H₆₇N₁₂O₇⁺, found 1007.88. ¹H-NMR (400 MHz; DMSO-d6): δ 10.58 (s, 1H), 8.56 (dd, J=7.7, 4.0 Hz, 1H), 8.44 (d, J=8.2 Hz, 4H), 8.24-8.15 (m, 1H), 8.02-7.88 (m, 5H), 7.83-7.71 (m, 4H), 7.53 (d, J=8.0 Hz, 2H), 7.25-7.21 (m, 1H), 6.87-6.81 (m, 2H), 5.73-5.68 (m, 1H), 4.41 (t, J=5.7 Hz, 2H), 3.66 (q, J=5.8 Hz, 3H), 3.62-3.56 (m, 3H), 3.55-3.46 (m, 22H), 3.13-3.06 (m, 4H), 2.43 (d, J=6.2 Hz, 2H), 2.32 (dd, J=7.9, 4.3 Hz, 2H), 1.74-1.67 (m, 2H), 1.29-1.18 (m, 3H), 1.08-0.93 (m, 3H). ¹³C NMR (101 MHz; DMSO-d6): δ 170.87, 170.74, 170.59, 165.8, 165.5, 158.6, 145.4, 130.7, 128.4, 128.2, 127.9, 70.22, 70.13, 70.03, 69.98, 67.3, 42.3, 36.63, 36.56.

### Example 3—Synthesis of Fluorescent rTCO FAST Probes for Antibody Labeling

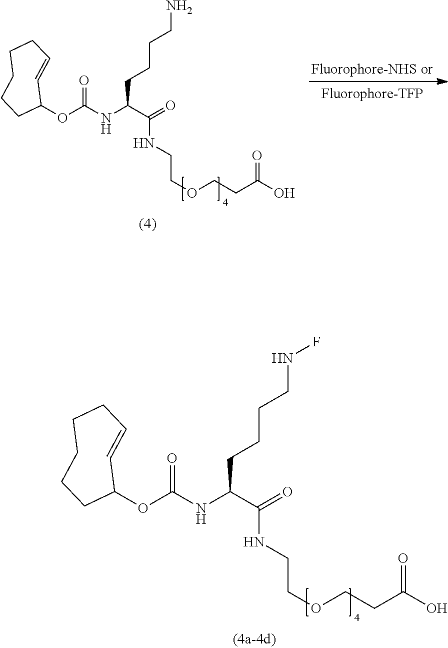

### Preparation of FAST-AF488 (4a)

A solution of (4) (2 mg, 3.8 μmol) in anhydrous DMSO (100 μL) was added to 2 mg of AFDye 488-TFP (2.93 μmol) in a 1 mL polyethylene screw-cap vial, as supplied by the dye manufacturer. DIPEA (2.5 equivalents, 1.26 μL) was then added and the mixture was vortexed until the dye had fully dissolved. After 10 minutes at room temperature the reaction mixture was loaded onto a Biotage SNAP Bio C18 column and the product was obtained after reversed phase chromatography (ammonium formate (2.5 mM, pH 4.5)/MeCN gradient elution) and rotary evaporation as a glassy orange solid. This material was dissolved in methanol (200 μL) and loaded onto a Waters tC18 Sep Pak that had been conditioned with methanol and equilibrated with 95:5 H₂O:MeOH. The Sep Pak was washed with 4 cartridge volumes of water and then eluted with methanol. The product was dried by rotary evaporation to a glassy intensely-orange film, and then further dried by serial dissolution and rotary evaporation with anhydrous 1:1 MeOH:toluene to give a pale orange dry crystalline powder lining the vial. (1.24 mg, 39%). MS [M−2H]⁻ calcd. 1060.32 for C47H58N5019S2, found 1060.59.

### Preparation of FAST-AF555 (4b)

A solution of (3) (1.13 mg, 2.08 μmol) in anhydrous DMSO (200 μL) was added to 2 mg of AFDye 555-NHS (1.6 μmol, MW 1247.64 as the triethylammonium salt) in a 1.5 mL eppendorf tube. DIPEA (4 equivalents, 1 μL) was then added and the mixture was vortexed until the dye had fully dissolved. After 15 minutes at room temperature the reaction mixture was loaded directly onto a Biotage SNAP Bio C18 column and the product was obtained after buffered reversed phase chromatography with ammonium formate (2.5 mM, pH 8.4)/MeCN gradient elution. After rotovap, the product was a glassy red solid coating the vial. This material was dissolved in methanol (200 μL) and loaded onto a 500 mg bed volume Waters tC18 Sep Pak that had been conditioned with methanol and equilibrated with 95:5 H₂O:MeOH. The Sep Pak was washed with 4 cartridge volumes of water and then eluted with methanol. The product was dried by rotary evaporation to a glassy-red/pink film, and then further dried by serial dissolution and rotary evaporation with anhydrous 1:1 MeOH:Toluene to give a pale pink dry crystalline powder lining the vial. (1.04 mg, 47%). MS [M]⁺ calcd. 1374.54 for C61H92N5022S4+, found 1374.71.

### Preparation of FAST-AF594 (4c)

A solution of (3) (2.1 mg, 3.8 μmol) in anhydrous DMSO (200 μL) was added to 2 mg of AFDye 594-NHS (2.9 μmol, MW 819.85) in a 1 mL polyethylene screwcap vial. DIPEA (2.5 equivalents, 1.26 μL) was then added and the mixture was vortexed until the dye had fully dissolved. After 10 minutes at room temperature the reaction mixture was loaded directly onto a Biotage SNAP Bio C18 column and the product was obtained after reversed phase chromatography (Ammonium Formate (2.5 mM, pH4.5)/MeCN gradient elution) as a glassy purple solid coating the vial. This material was dissolved in methanol (200 μL) and loaded onto a Waters tC18 Sep Pak that had been conditioned with methanol and equilibrated with 95:5 H₂O:MeOH. The Sep Pak was washed with 4 cartridge volumes of water and then eluted with methanol. The product was dried by rotary evaporation to an intense purple film, and then further dried by serial dissolution and rotary evaporation with anhydrous 1:1 MeOH:Toluene to give a reddish-purple dry crystalline powder lining the vial. (1.46 mg, 40%). MS [M−2H]⁻ calcd. 1248.47 for C61H78N5019S2, found 1248.77.

### Preparation of FAST-AF647 (4d)

A solution of (3) (1.28 mg, 2.3 μmol) in anhydrous DMSO (100 μL) was added to 2.3 mg of AFDye 647-NHS (1.8 μmol, MW 1273.7 as the triethylammonium salt) in a 1.5 mL eppendorf tube. DIPEA (2.5 equivalents, 1.26 uL) was then added and the mixture was vortexed until the dye had fully dissolved. After 10 minutes at room temperature the reaction mixture was loaded directly onto a Biotage SNAP Bio C18 column and the product was obtained after reversed phase chromatography (Ammonium Formate (2.5 mM, pH4.5)/MeCN gradient elution) as a glassy royal blue coating the vial. This material was dissolved in methanol (200 uL) and loaded onto a Waters tC18 Sep Pak that had been conditioned with methanol and equilibrated with 95:5 H₂O:MeOH. The Sep Pak was washed with 4 cartridge volumes of water and then eluted with methanol. The product was dried by rotary evaporation to an intense cobalt-blue film, and then further dried by serial dissolution and rotary evaporation with anhydrous 1:1 MeOH:Toluene to give a dry blue crystalline powder lining the vial. (1.39 mg, 55%). MS [M]⁺ calcd. 1400.53 for C63H94N5022S4, found 1400.94.

FIG. 16A contains chemical structures of FAST-AF488 (4a) and FAST-AF555 (4b). FIG. 16B contains chemical structures of FAST-AF594 (4c) and FAST-AF647 (4d).

### Example 4—TSTU-ENBA Activation Method

General procedure

To a 500 μL Eppendorf tube containing 10-30 μL of 4(a-d) in anhydrous DMSO were added 2 equivalents of DIPEA followed by 4 equivalents of N,N,N′,N′-Tetramethyl-O—(N-succinimidyl)uronium tetrafluoroborate (TSTU) and the solution was quickly vortexed after capping the tube. After one minute, 5 equivalents of 4-(ethylamino)-butanoic acid hydrochloride (ENBA) were added followed by an additional 5 equivalents of DIPEA. Note: The reaction time prior to addition of ENBA is concentration dependent and may be complete in as little as 10 seconds at higher dye/TSTU concentrations. For a dye solution between 1-2 mM, the sixty second reaction time provides optimal discrimination between the PEG₄ carboxylic acid and the rhodamine 2′-carboxylic acid, with minimal conversion to the double-NHS species. After addition of ENBA, the solution was again vortex-mixed and ready to use for protein labeling or LCMS analysis after thirty seconds. Routine analyses during method development revealed quantitative (>95%) probe activation.

### Preparation of FAST-AF488-NHS

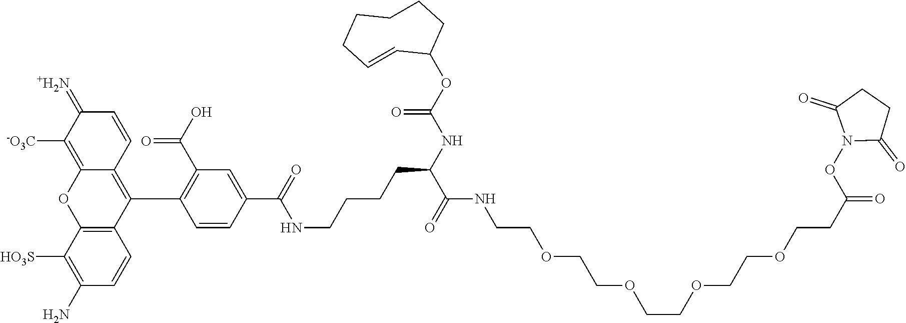

Representative example of AF488-rTCO-P₄ activation was prepared as follows. The chemistry is moderately moisture sensitive due to the risk of NHS hydrolysis and appropriate storage/handling conditions were applied (as described below). Stock solutions of the reagents were prepared as tabulated: TSTU was prepared fresh for each batch of FAST probe activation; ENBA and DIPEA solutions were stable at room temperature or stored cold.

TABLE 1

| | | | Typical | |
|---|---|---|---|---|
| | | | stock | |
| Reagent | Solvent | Molar eq. | conc. | Notes |
| FAST | DMSO | 1 | 1-2 | Stored at −20 to −80° C. in a |
| probe | | | mM | secondary container with |
| | | | | desiccant to avoid freeze-thaw |
| | | | | condensation. |
| DIPEA | DMF | (stage 1) 2 | 200 | Stable at RT |
| | | (stage 2) 5 | mM | Insufficiently miscible in DMSO |
| TSTU | DMSO | 4 | 200 | 6-10 mg aliquots can be weighed |
| | | | mM | out in advance, stored at 4° C. |
| | | | | with desiccant and dissolved to |
| | | | | target concentration at each use |
| ENBA× | DMSO | 5 | 200 | Stable at RT |
| | | | mM | |

### Example 5—Click-Quenching Kinetic Assays

A. Sample Preparation

A stock solution BHQ3-Tz was prepared at a concentration of 1 mM in DMSO and the concentration validated by absorbance measurements on serially diluted samples (extinction coefficient at peak, 42700 M⁻¹ cm⁻¹ in PBS pH 7.3). This parent solution was stored at −80° C. At the time of kinetic assays, aliquots of the stock solution were further diluted 50-fold into DMSO, for a final concentration of 20 μM BHQ3-Tz.

Individual stock solutions of FAST probes in DMSO (1-2 mM) were diluted into pH 9 bicarbonate buffer (MicroEssential Laboratory) to prepare a stock solution at a concentration of 2-10 nM immediately prior to assays. 2 mL of this solution was added directly into fresh disposable fluorescence cuvettes (10×10 mm) and allowed to equilibrate to 25° C. in the instrument prior to beginning the assay.

FAST labeled antibodies were stored in PBS at 4° C. at concentrations of 5-15 μM after labeling. Because nonspecific adsorption to the disposable polystyrene cuvettes was observed (declining signal after addition of the antibody), 2 mL of pH 9 buffer were added to the cuvette followed by addition of 40 μL of 2 mg/mL unlabeled cetuximab (40 μg/mL) as a blocker. The sample was allowed to stir in the cuvette for a minimum of 60-90 seconds prior to addition of the FAST-labeled antibody. The FAST-labeled antibodies were then were diluted directly into the blocked cuvette to achieve a final concentration between 2-10 nM.

B. Fluorimeter acquisition The QM-400 spectrophotometer was equipped with a Pelletier apparatus for thermal control (25° C.) and integrated magnetic stirring. Time-based acquisitions were initiated at appropriate dye-specific wavelengths and structured to sequentially capture the baseline emission of the buffer solution, the initial brightness of the dye solution, and the time course of quenching in a continuous trace. Data were acquired at a rate of 3 or 5 points per second. After measuring the initial fluorescence signal, 5-50 μL of the BHQ3-Tz solution were added to the cuvette via the instrument's sample addition port and data acquisition continued until the quenching reaction was complete.

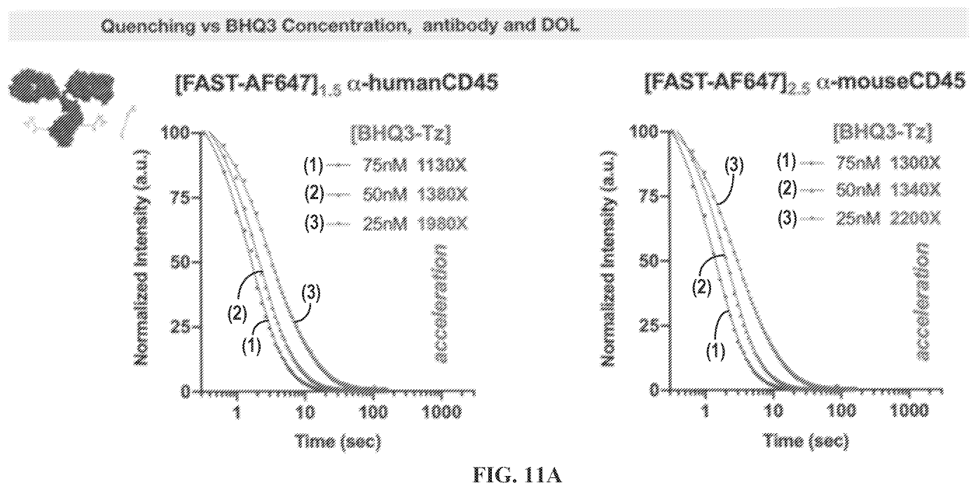

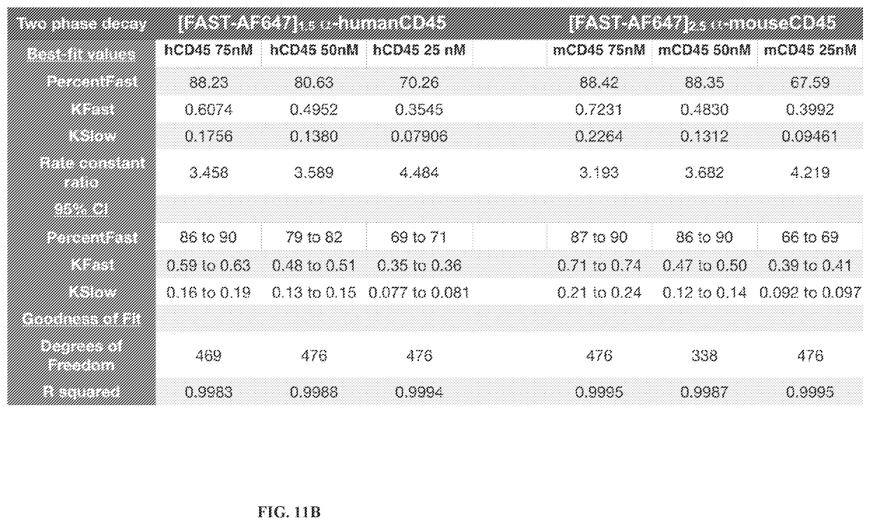

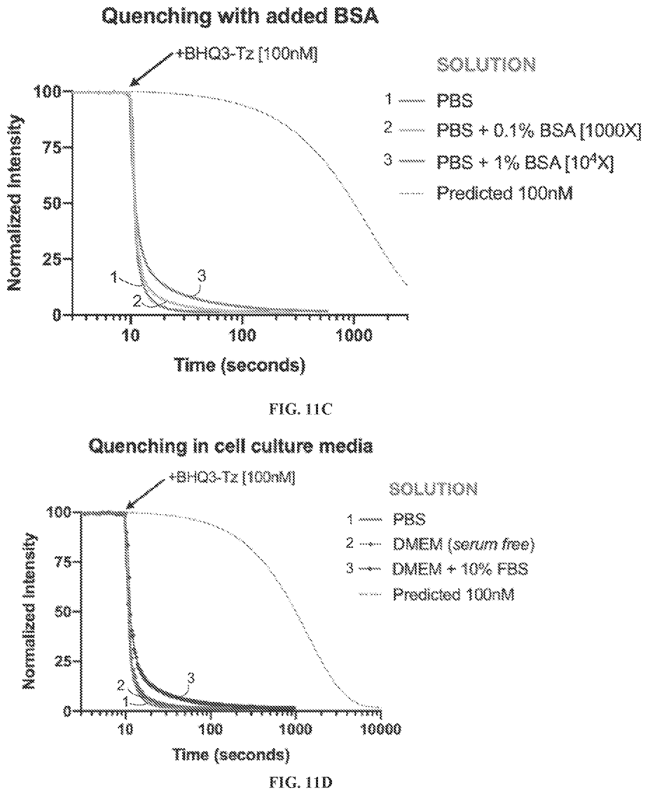

C. Kinetic fitting Data were analyzed in GraphPad Prism 8.3 (Graphpad Software). Quenching kinetics for the reaction between FAST probes (free label in solution) and BHQ3-Tz were fitted to a single exponential decay, with the time of BHQ3-Tz addition set to t=0 for plotting/fitting purposes (Part D, following page). FIG. 2B includes an example of the time course before and after addition. Second order rate constants for the reaction of BHQ3-Tz and the individual FAST probes were calculated from the plots of observed rate vs BHQ3-Tz concentration, per routine. For FAST-labeled antibodies, curves were fitted to a double exponential (two phase) decay and the rate constants were reported as in FIGS. 11A and 11B.

### Example 6—Tz-TCO Based Antibody Labeling and Quenching

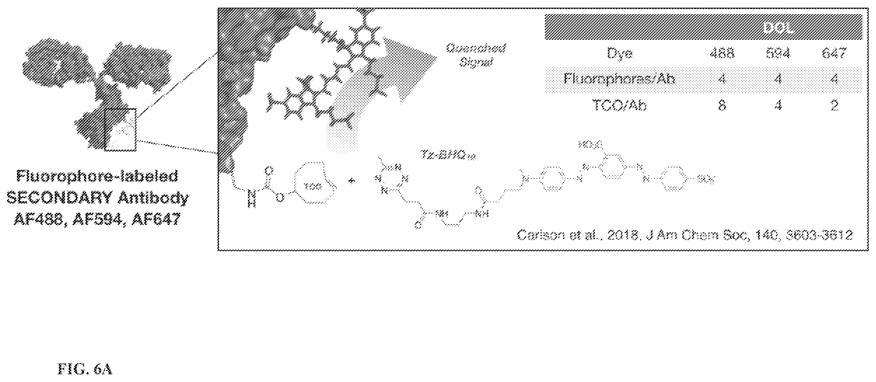

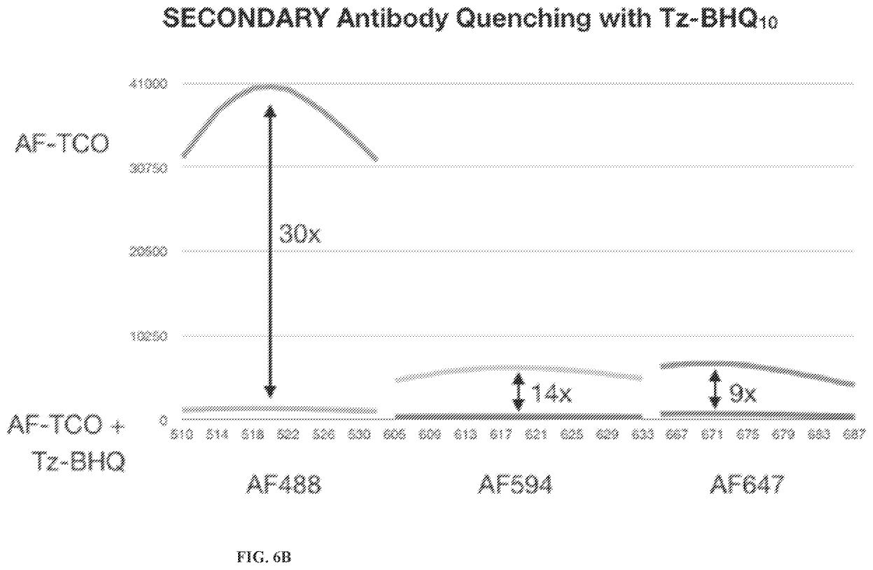

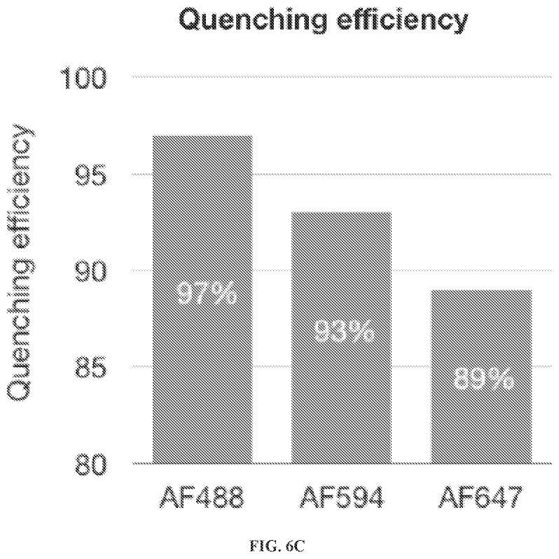

As the working examples demonstrate, the technology described herein involves efficient, fast and maximum quenching of antibody-associated fluorochromes of different wavelengths (See FIGS. 1A, 1, and 1C). This is achieved via a modular linker between fluorochromes and antibodies with an embedded TCO for clicking with a tetrazine-quencher (See FIG. 1A). In a proof of principle study, we labeled a commercially available Alexa-Fluor 488 labeled secondary antibody with TCO (4-5 TCO/antibody) and observed a marked reduction in antibody fluorescence after click-reaction with the BHQ10-tetrazine (FIGS. 6A, 6B, and 6C).

The working examples provide a series of TCO-fluorophore reagents built around lysine as a ternary scaffold, equipped with a linker (e.g., PEG₄ linker) for antibody conjugation (FIG. 1A). One embodiment of the disclosure is the use of axial 3-OH-functionalized TCO (release-TCO, rTCO), which has enhanced stability under a range of biochemical exposures, for further testing. [See Ref 15]

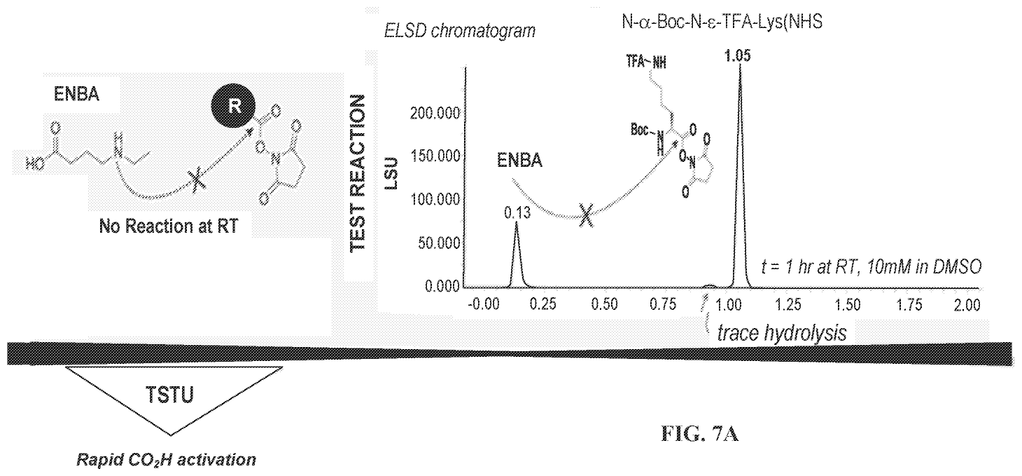

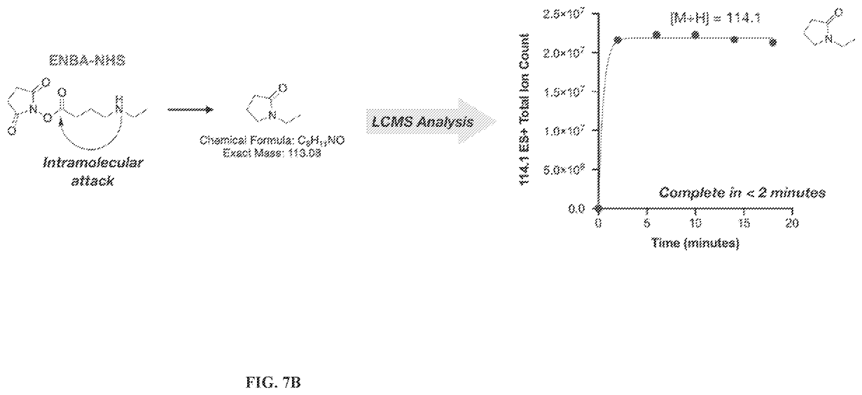

The examples describe isolation of TCO-fluorophore-activated esters under gently buffered reverse phase conditions (pH 4.5-5) for conjugation to biological molecules such as antibodies. However, a new method is also developed for rapid microscale activation of the TCO-fluorophore reagents immediately prior to antibody conjugation (FIGS. 1A, 7A, and 7B). The negligible reactivity of secondary amines with NHS esters was exploited to neutralize excess activating reagents, such as TSTU (N,N,N′,N′-Tetramethyl-O—(N-succinimidyl)uronium tetrafluoroborate). Rapid intramolecular ring closure converts 4- (ethylamino)butanoic acid (ENBA) succinimidyl ester to inert N-ethyl-2-pyrrolidone to quench the reaction with no impact on the TCO-fluorophore-NHS. This allows use of TSTU in a significant molar excess to i) achieve rapid conversion of the TCO-fluorophore to the NHS ester (seconds); ii) reduce the impact of trace amounts of water on reliable dye activation; iii) simplify microscale reagent titration for applications in a biological context. Importantly, this approach also enables kinetic discrimination between the PEG₄ carboxylic acid and the comparatively hindered 2′-carboxylic acid on many xanthene dyes (e.g. AF488, AF594). [See Ref. 17] The resulting dye solution contains no competing reactive species (i.e., no residual activator or contaminating active esters that could react with the antibody) and can be aliquoted for immediate use or stored (≤−20° C.) for subsequent labeling reactions.

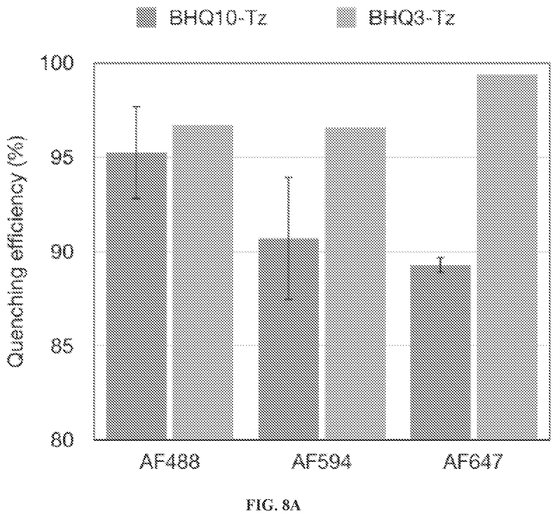

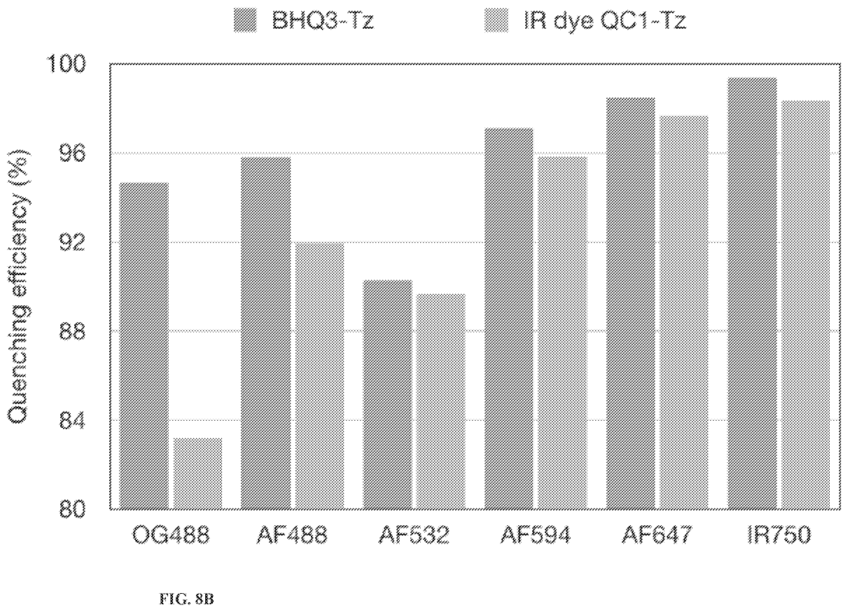

Three different types of quencher (BHQ10, BHQ2, BHQ3, and IRDye QC-1 [See Ref 18]) were tested with relevant fluorophores. (BHQ2 probes were made but found insufficiently soluble). FIG. 1B shows results of the experiments with BHQ3 (FIG. 1B), which showed high quenching efficiency with all the dyes that were tested (FIGS. 8A and 8B). From the broad wavelength compatibility, a significant contribution from static/contact quenching can be inferred, which does not require spectral overlap between the fluorophore and quencher. [See Ref 19] The reagents within the present claims provide adequate aqueous solubility (≤25 μM in PBS) to achieve rapid quenching given the expected click reaction kinetics. Furthermore, the linker structure is compatible with cooperative quenching, such that a BHQ3-Tz tethered to one TCO-FI site may be within quenching range of adjacent dye molecules. Exemplary HTz-PEG₅-NHS linker was tested in the working examples, which conferred sufficient solubility and linker length (rendered to scale in an extended conformation, FIG. 1C).

Ultra-fast and highly efficient quenching. A series of experiments were performed to quantitate: i) the labeling performance of the conjugates, including degree of labeling (DOL) per dye equivalent (the antibody labeling efficiency of the TCO-F1 conjugates); ii) antibody brightness as a function of DOL; iii) effect of the TCO-Tz quenching method on different fluorochrome-modified antibodies across the visible wavelength spectrum (FIG. 2). The FAST linkers within the present claims displayed excellent labeling characteristics with all the fluorophores and antibodies tested, with a predictable and efficient DOL as a function of dye concentration. Mouse splenocytes stained with a FAST647 anti-CD4 antibody matched or exceeded the staining performance and brightness of a conventional commercial standard (FIG. 2A). The brightness of FAST labeled antibodies was substantially quenched upon treatment with BHQ3-Tz: pre/post fluorescence emission spectra of a FAST647-labeled anti-EGFR antibody (cetuximab) illustrate a >99% decrease of the dye emission (FIG. 2B). The quenching dynamics were assessed in the cellular/imaging context, staining A431 cells with the respective cetuximab conjugates and collecting images before and after a three minute incubation with 20 μM BHQ3-Tz at pH 9 (FIG. 9). Quenching was very efficient for the far red dye AF647, which has the greatest degree of spectral overlap with BHQ3, but the quantitative reduction in brightness was similar for different fluorochromes spanning the visible range, with a general trend toward superior quenching at higher DOL. To further delineate the dynamics of the rTCO/aryl-HTz ligation and assess any impact from release (which would liberate the quencher from the antibody), the brightness of FAST647-Cetuximab/BHQ3-Tz quenching was measured over a four hour time course as a function of pH (see FIG. 2C). Addition of BHQ3-Tz produced a uniform ˜99% reduction in the baseline fluorescence intensity, irrespective of pH. At pH 5-6, the fluorescence rebounded to approximately 15% of the initial intensity, with a faster rate at the lower pH, indicative of partial release of the quencher. In contrast, the net rebound in brightness was minimal at physiologic pH (≤5% in PBS, pH 7.4), and negligible at pH 9, with no significant change in the observed quenching after 4 hours.

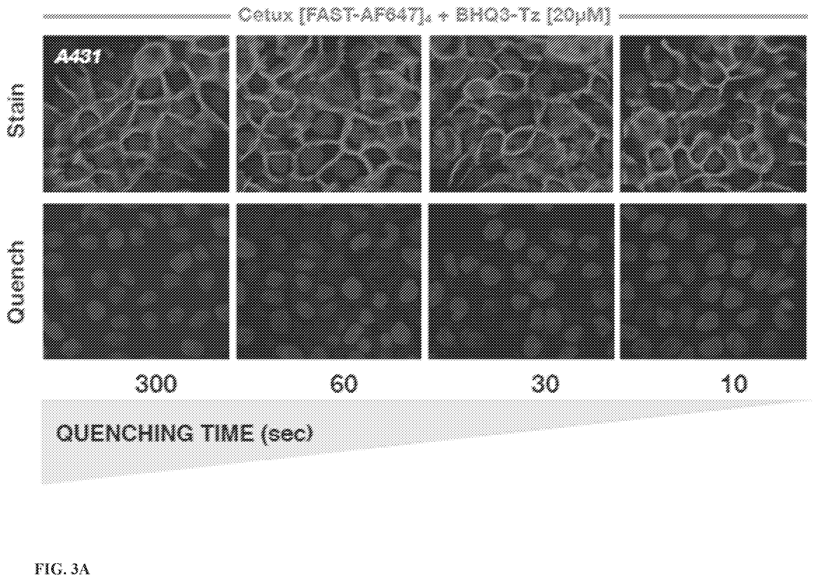

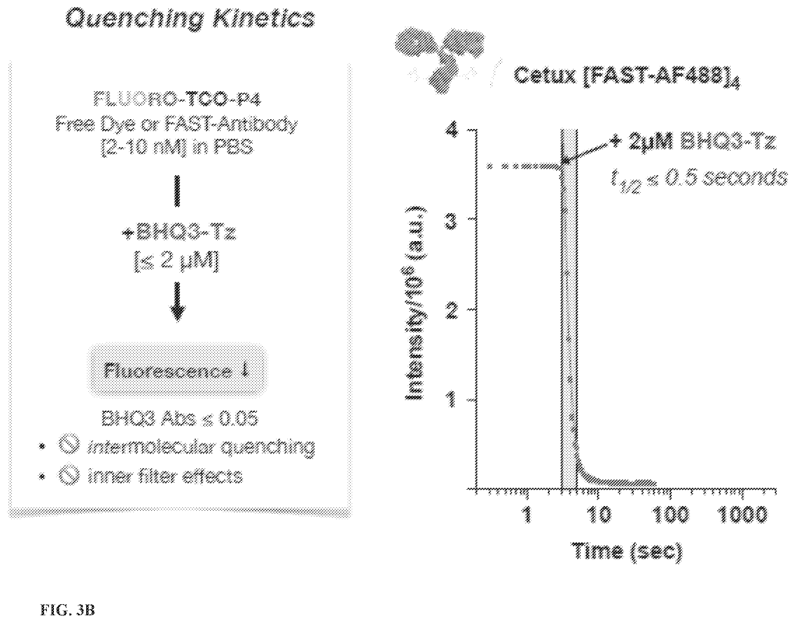

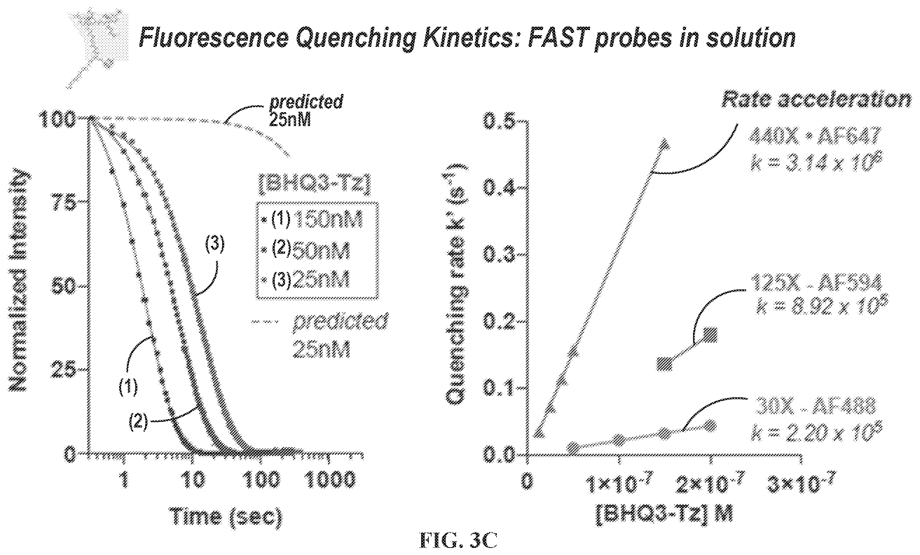

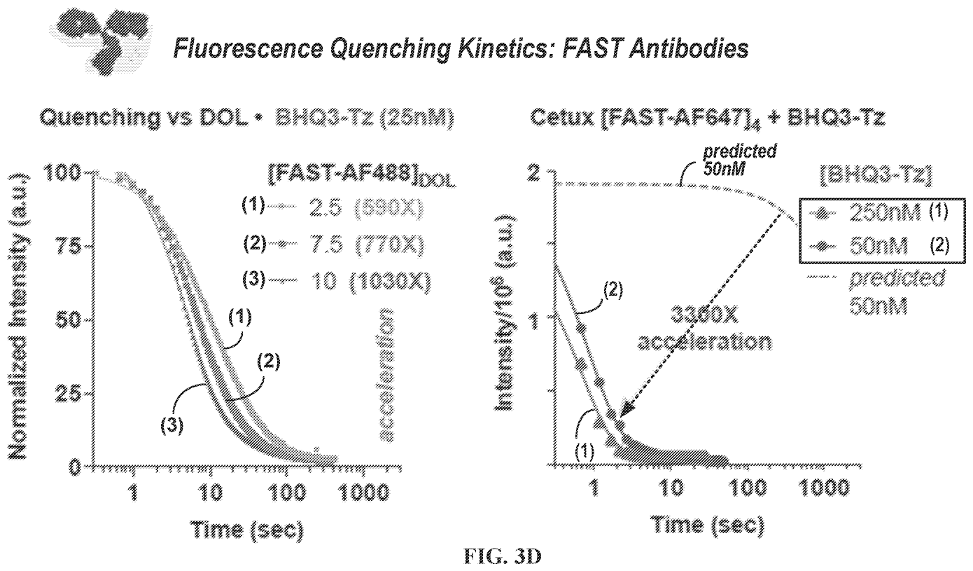

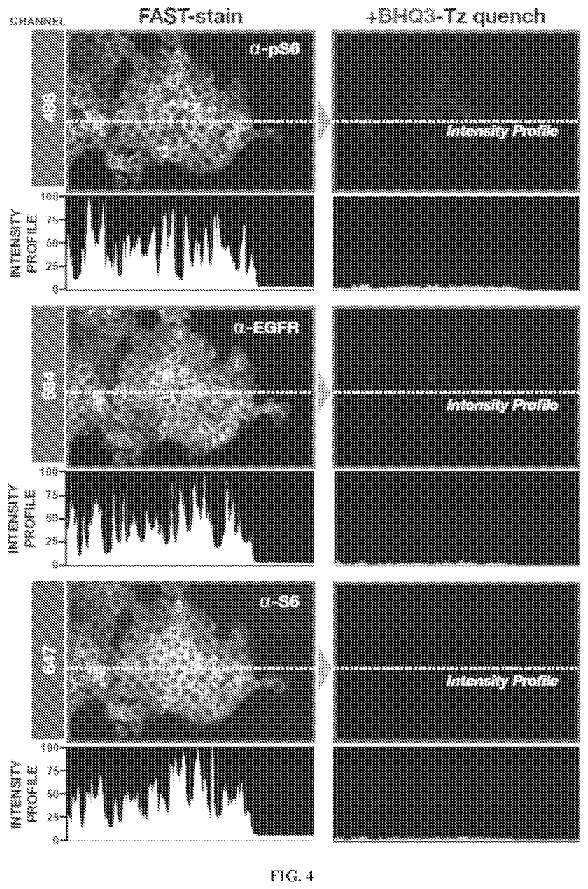

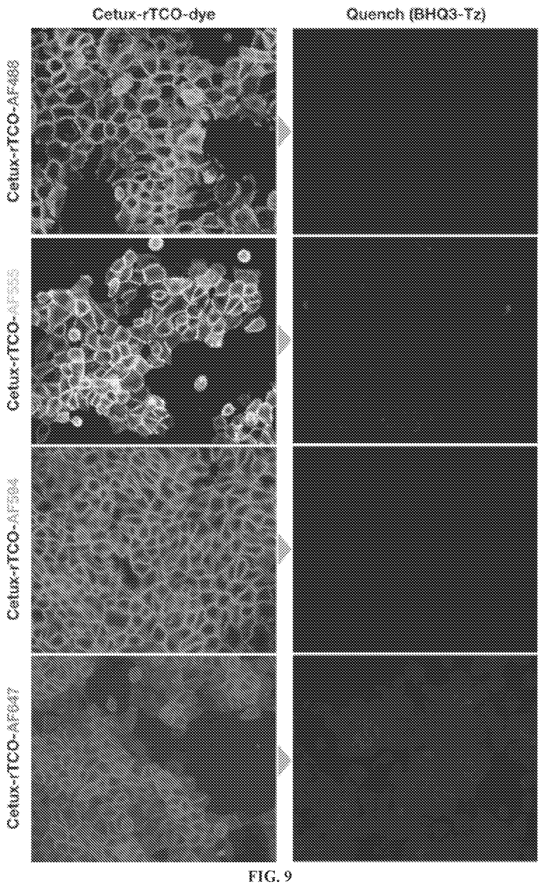

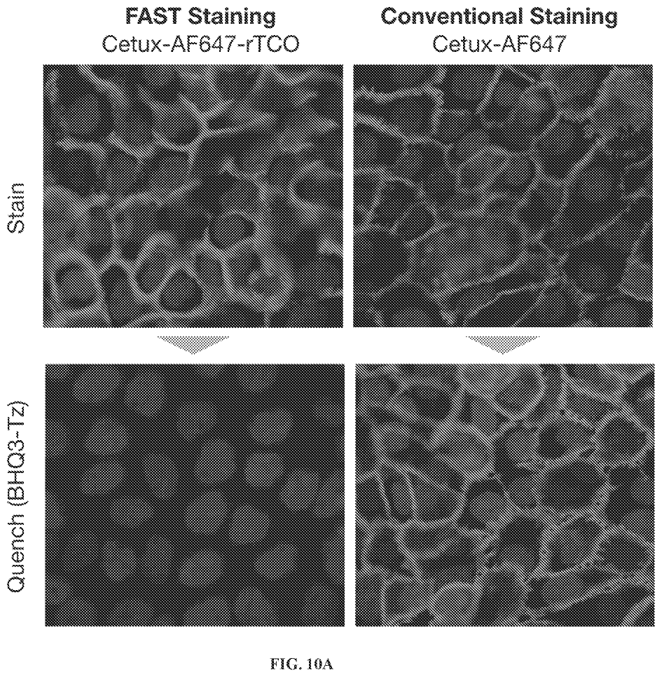

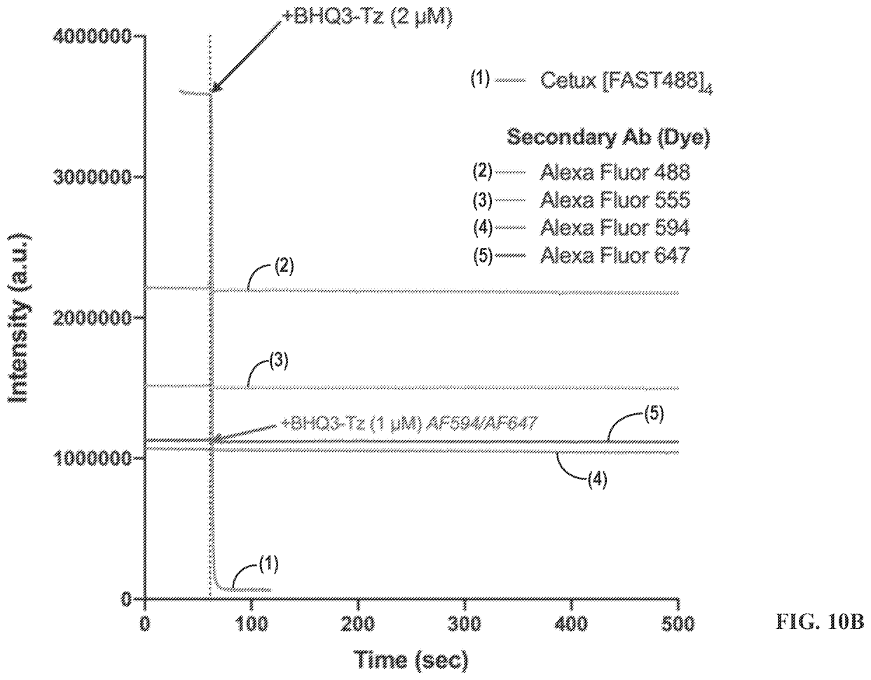

Having established the desired magnitude of quenching, the minimum incubation time required to achieve high (>98%) quenching efficiency after addition of BHQ3-Tz (20 μM) to stained cells was determined. The fast click kinetics of the benzylamino-H-Tz with rTCO (k=7200 M⁻¹s⁻¹ in PBS at 25° C.) enabled rapid quenching within a minute (FIG. 3A). Surprisingly, equally efficient quenching was observed when the BHQ3-Tz incubation time was progressively decreased to as little as ten seconds, as short as experimentally feasible. Importantly, conventionally stained cells exhibited no discernible change in brightness when exposed to BHQ3-Tz under these conditions (FIG. 10A).

A fluorimeter-based assay was implemented to directly observe the click kinetics for BHQ3-Tz and FAST-labeled antibodies/probes. At nanomolar fluorophore concentrations this allows real-time observation of pseudo first-order click rates with negligible optical interference, provided the BHQ3-Tz concentration in the cuvette is kept appropriately low.

Addition of BHQ3-Tz produced no effect on the brightness of conventionally-labeled secondary antibodies (FIG. 10B); in contrast, at a matched BHQ3-Tz concentration of 1-2 μM (≤ 1/10th the amount used in the initial imaging experiments) it was found that FAST-AF488 antibody fluorescence was quenched remarkably rapidly, with a half-life of less than half a second (FIG. 3B). Adding a 100-fold excess of unlabeled antibody (relative to FAST-Ab, used to block nonspecific adsorption to the cuvette) produced no effect on the rate, suggesting that the kinetics are not being driven by BHQ3-protein interactions. Given the multivalency of antibody labeling, these rates reflect a cumulative multi-click quenching process, averaged across multiple FAST label (dye-TCO) pairs, not a single chemical event. To better assess the intrinsic rate constants, the quenching rate for the free FAST probes in solution was assessed as a function of BHQ3-Tz concentration. Click rates were dye-dependent, exhibited classical concentration-dependent (pseudo first order) kinetics, and were again dramatically accelerated relative to the parent Tz-TCO (FIG. 3C). Measured second-order rate constants ranged from −30-fold faster than expected for FAST-AF488 to nearly 440-fold faster than expected for FAST-AF647.

When these methods were extended to reassess kinetics for the BHQ3-Tz reaction with FAST-labeled antibodies, even more extreme acceleration of the quenching rates was unexpectedly observed, rising in tandem with increasing DOL (FIG. 3D). The fluorescence of the AF488- labeled Ab was quenched with an apparent second order rate constant as high as 7.4×10⁶ M⁻¹s⁻¹ (t_{1/2}=3.7 s at 25 nM BHQ3-Tz, ˜1000-fold accelerated). Mirroring the trend for the free dyes, the apparent quenching rate for the AF647-labeled Ab was even faster at 2.4×10⁷ M⁻¹s⁻¹ (t_{1/2}=0.6 seconds at 50 nM BHQ3-Tz), accelerated by >3300-fold compared the expected bimolecular click rate. Concordant rate accelerations and DOL/concentration dependence were observed for additional control antibodies, confirming the generality of this effect (FIGS. 11A and 11B). While the free FAST probes exhibited excellent single exponential fits under pseudo first order conditions (FIG. 3C), the FAST-antibody quenching was better fit to a double exponential. Neither did the observed rate vary linearly with quencher concentration, with the apparent second order rate rising as the concentration of BHQ3-Tz decreased, suggestive of a cooperative quenching interaction between dyes and in keeping with the observed accelerating effects of DOL (FIGS. 11A and 11B). While the efficiency of quenching in the biomolecular context is evident from the imaging experiments, to further explore the general biocompatibility of the FAST-acceleration the reaction kinetics between FAST-labeled antibodies and BHQ3-Tz were measured in PBS with added BSA (to further assess the impact of high protein concentration) and in cell culture media. Click/quenching rates remained profoundly accelerated, with only subtle changes in the temporal profile (FIGS. 11C and 11D).

Intramolecular contact quenching for fluorophore-BHQ pairs is well-known for dual-labeled oligonucleotide [See Ref 20] and peptide [See Ref 21] cleavage probes. In the oligonucleotide context where this has been studied more quantitatively, fluorophore-quencher interactions subtly perturb melting temperatures [See Ref. 19] and can promote intramolecular dimer formation with an affinity sufficient to form a stemless hairpin. [See Ref 22] Without being bound by theory, it is believed that dye-dependent fluorophore-BHQ3 interactions drive a reversible molecular association of sufficient affinity/duration to promote Tz-TCO ligation, even at nanomolar concentrations. The temporal dynamics of this process are intriguing, given that no fluorescence quenching (a process occurring on the nanosecond timescale) is observed in the absence of the click reaction.

### Example 7—Imaging of Multiple Targets in Single Cancer Cells

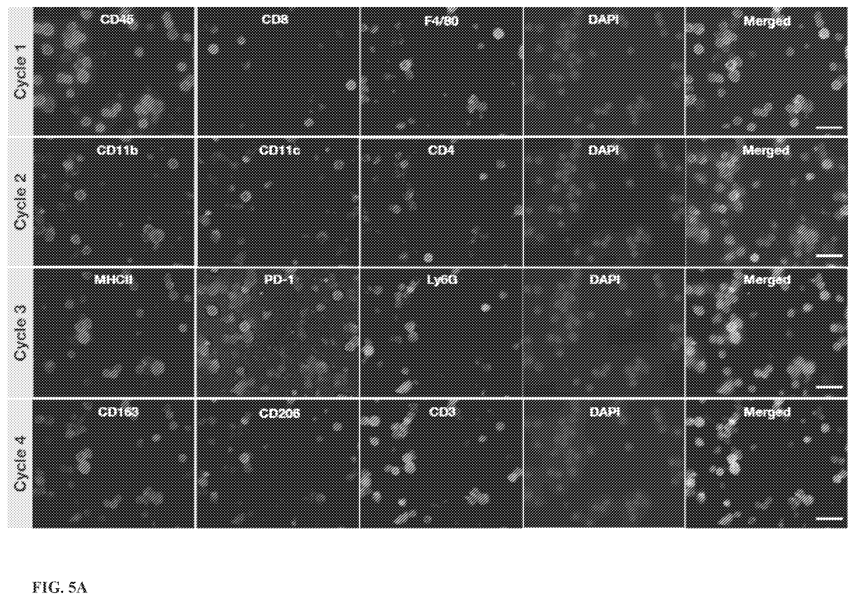

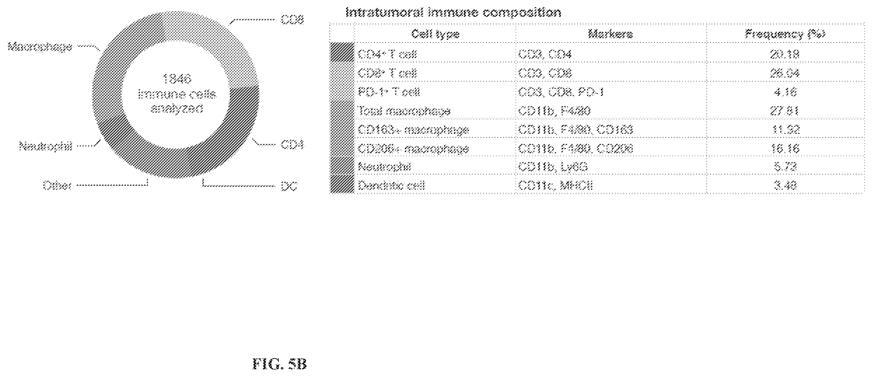

To move from single-channel benchmark studies to the clinically relevant objective of imaging/quenching multiple protein markers within individual cancer cells, multichannel FAST staining was next validated. The targets of known importance in cancer diagnosis and complementary spatial distributions, including cell surface, cytoplasmic, and nuclear markers, were selected. In one illustrative example, A431 epidermoid cancer cells for EGFR, S6 and phospho-S6 (pS6), were stained with three antibodies simultaneously and imaged the same field of view before and after quenching with BHQ3-Tz (FIG. 5). Quantitative intensity profiles for each channel span the stained and quenched images to allow visualization of the quenching efficiency, which proved to be excellent across all cellular compartments. The residual background signal in the quenched images is <5% of the peak staining intensity, inclusive of channel-specific auto fluorescence (higher at green wavelengths than red). Given the complexity of normalizing absolute fluorescence intensity between individual clinical samples, quantitative co-expression and the ratio of paired markers (e.g. pS6/S6 as demonstrated here) are particularly important for both cellular classification and as a tool to measure therapeutic protein inhibition. [See Ref 10] With a working method for multi-color cycling, co-expression of multiple protein markers was profiled in a murine model of clinical FNA samples.

### Example 8—Tumor Immune Cell Profiling by Cyclic Imaging

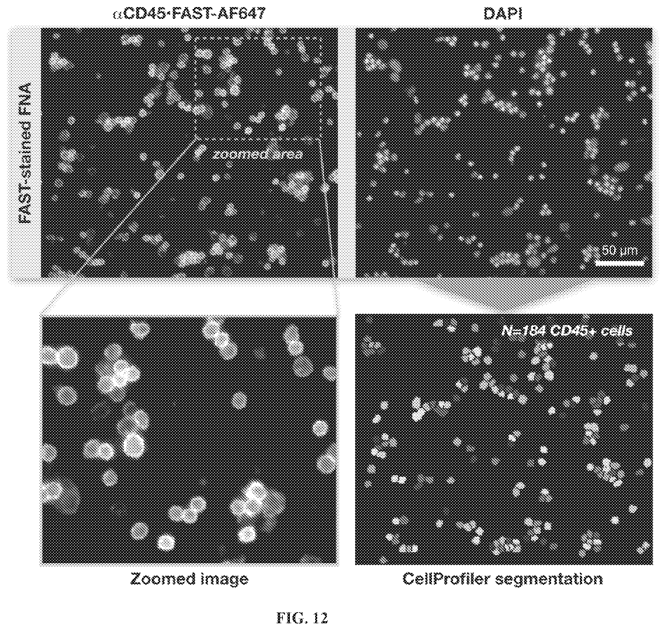

To show applicability of the method for tumor immune cell profiling, 12 immune markers were imaged (CD45, CD8, CD3, CD4, PD-1, CD11b, F4/80, MHCII, CD163, CD206, Ly6G, CD11c; FIG. 13) in cells directly harvested from MC38 mouse colon cancer, a highly immunogenic tumor model. [See Ref 23] FNA samples were obtained from subcutaneously implanted MC38 tumors (See general methods) and imaged in successive cycles of FAST staining. Cell nuclei were stained with DAPI for alignment of images across cycles. CD45 was imaged in cycle 1 as a pan-hematopoietic marker for identification of the immune cells in each field of view and selection of optimal imaging locations across the stained slide. In the FNA sample presented in FIG. 5, 11 different field of views with a sufficient number of cells were selected during cycle 1, and images of subsequent cycles were taken from the same positions to record the same set of cells. For image analysis, CD45+ immune cells were first identified among DAPI+ cells by cell segmentation of images to create a mask for all immune cells to analyze (FIG. 12). Image sets of each field of view were aligned using normalized cross correlation [See Ref 24], and the CD45 mask was applied to measure the fluorescence intensity of immune markers in each CD45+ cell. Cell Profiler was used for the image analysis. A total of 1846 CD45+ immune cells were detected from images of 11 fields of view. Distinct immune cell populations within CD45+ cells were identified based on their expression level of the immune markers (FIG. 5B). Thresholds for the positivity of a cell for each marker were determined from the fluorescence intensity distribution. The frequency of relevant immune cell populations (CD8+ T cells, CD4+ T cells, macrophages, dendritic cells, and neutrophils) was quantified as shown in FIG. 5B. The frequency of key immune population subsets, such as CD8+ T cells expressing immune inhibitory receptor PD-1, was also analyzed as an important target of cancer immunotherapy with immune checkpoint inhibitors. In addition, the expression levels of CD163 and CD206 in macrophages were assessed to analyze the immunosuppressive phenotype, which has been reported to be observed in tumor-associated macrophages. [See Ref. 25]

### Example 9—Synthesis of Fluorescent 5-OH TCO FAST Probes for Antibody Labeling

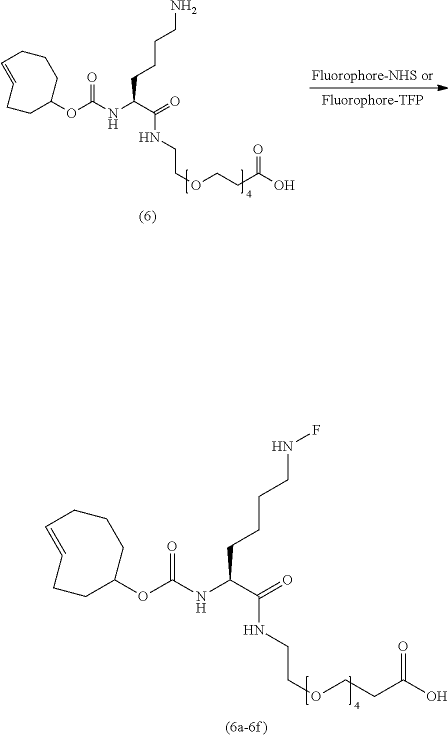

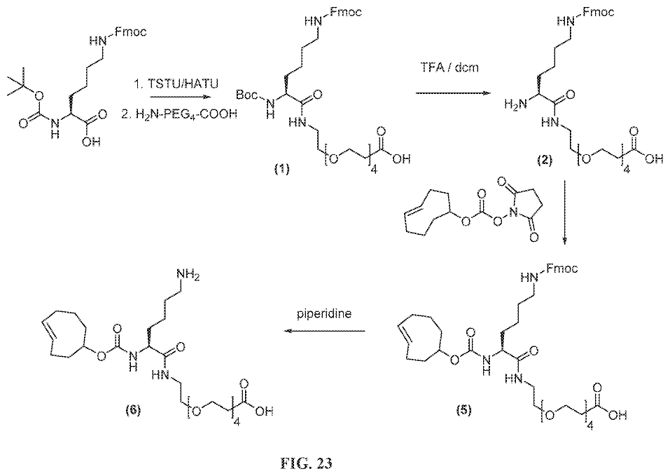

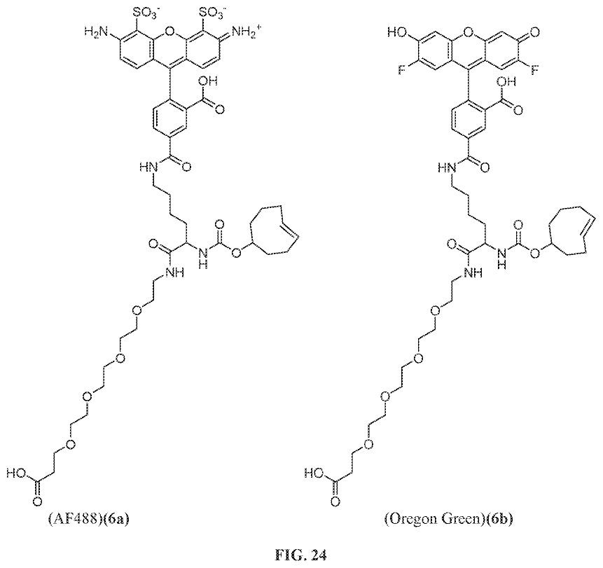

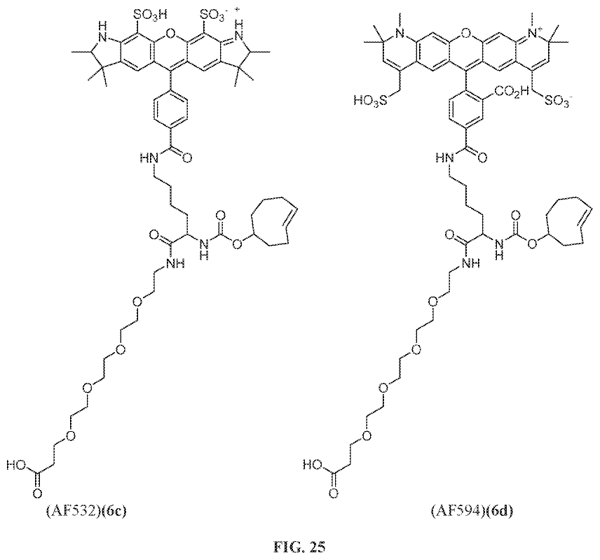

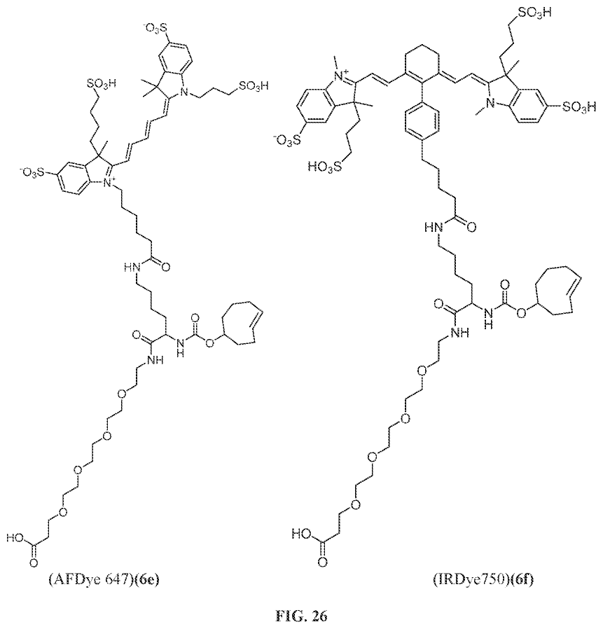

Synthesis of the key intermediate 6 is shown in FIG. 23. The intermediate 6 was prepared using methods and procedures similar to those described for intermediate (4) in Example 1. Compounds 6a-6f were prepared according to the methods and procedures similar to those described for compounds 4a-4d in Example 3. FIG. 24 contains chemical structures of FAST-AF488-TCO₁ (6a) and FAST-Oregon Green-TCO₁ (6b). FIG. 25 contains chemical structures of FAST-AF532-TCO₁ (6c) and FAST-AF594-TCO₁ (6d). FIG. 26 contains chemical structures of FAST-647-TCO₁ (6e) and FAST-IR750-TCO₁ (6f).

### Example 10—Synthesis and Evaluation of Fluorescent Double 5-OH TCO FAST Probes for Antibody Labeling

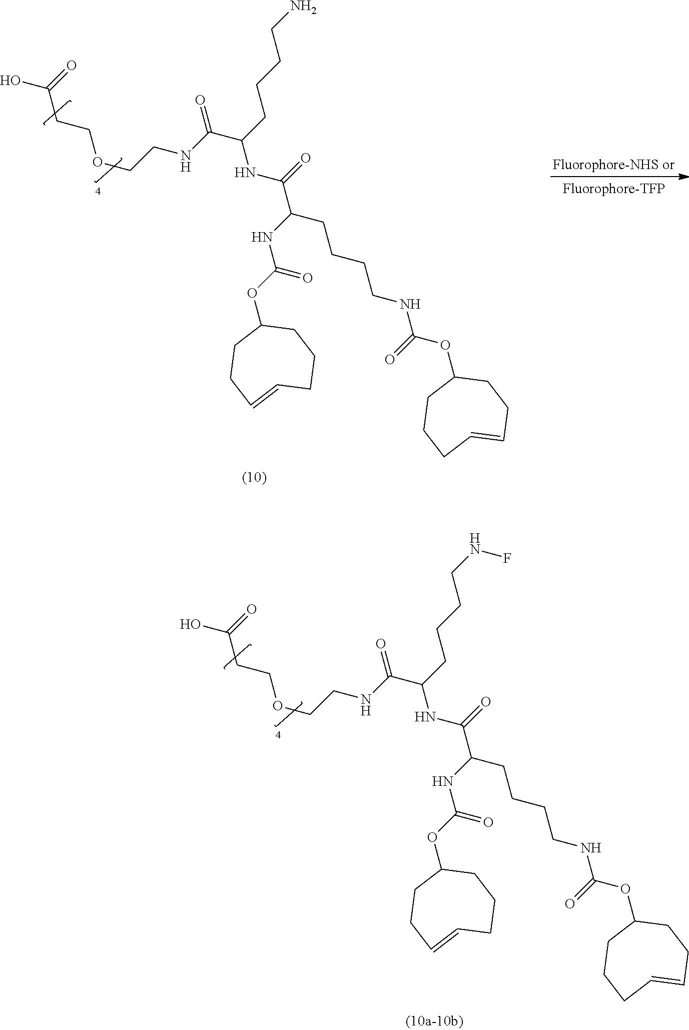

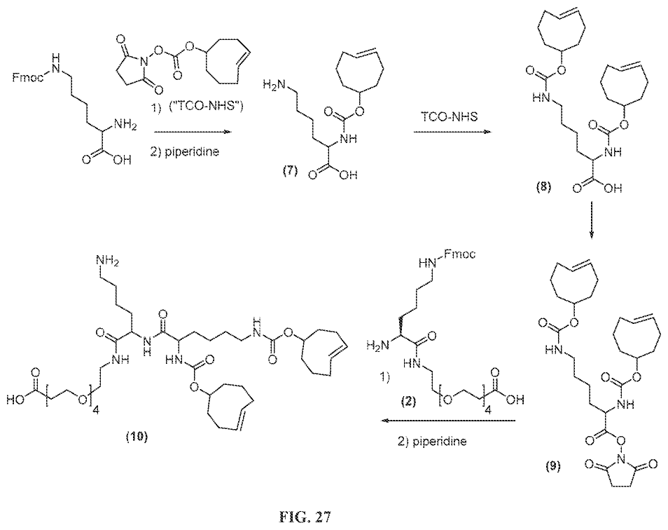

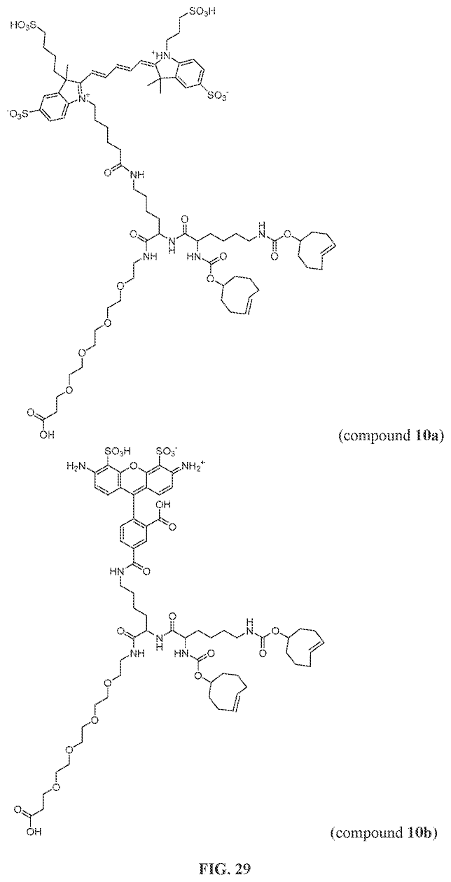

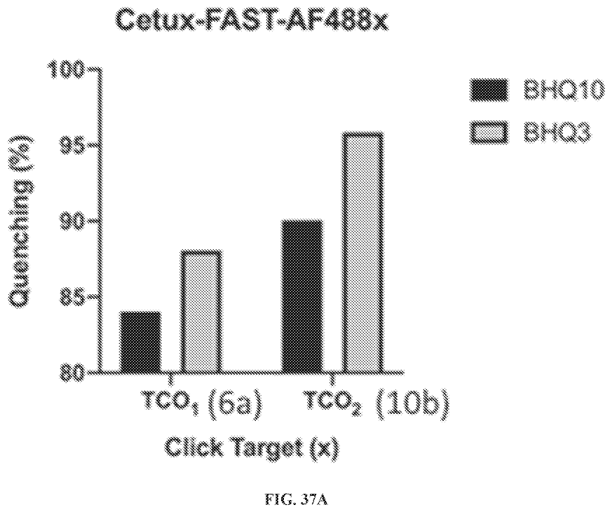

Synthesis of the key intermediate 10 is shown in FIG. 27. The intermediate 10 was prepared using methods and procedures similar to those described for intermediate (4) in Example 1. Compounds 10a-10b were prepared according to the methods and procedures similar to those described for compounds 4a-4d in Example 3. FIG. 29 contains chemical structures of FAST-AF647-TCO₂ (10a) and FAST-AF488-TCO₂ (10b). FIG. 26 contains chemical structures of NHS activated esters of FAST-AF647-TCO₂ (10a) and FAST-AF488-TCO₂ (10b). The results of imaging and quenching experiments using compound 10b are shown in FIGS. 37A and 37B, in comparison with the TCO₁ compounds 6a and 6e (as prepared in example 9).

Details of experiment described in FIG. 37A: Cetuximab (2 mg/mL in PB S-bicarb pH 8.4) was incubated with the NHS derivatives of compound 6a and 10b respectively. After labeling, the antibody was purified from the excess/free dye with a 40K zeba column. The labeled antibodies were then diluted into 100 μL of PBS (final concentration 200 nM) in a 96 well plate in triplicate. The baseline fluorescence emission spectrum of the antibody solution was measured with a TECAN Spark fluorimeter to determine the brightness at the peak emission wavelength. 0.5 μL of BHQ3-Tz (1 mM stock in DMSO) were then added to each well and the plate was allowed to incubate at room temperature for one minute. The fluorescence emission spectrum was then remeasured. The Quenching % is calculated from the brightness post/pre BHQ3-Tz. Quenching (%)=100(1−Intensitypost/Intensitypre).

Details of experiment described in FIG. 37B: Cultured A431 cells were fixed with 4% formaldehyde for 10 mins. In parallel, cetuximab was labeled with FAST probes (6a, 6e, 10b), prepared as their NHS derivatives: the antibody was mixed with 10 molar equivalents of the dye in PBS-Bicarb pH 8.4, then purified by 40K zeba spin column. Staining and imaging: The fixed cells were incubated for 20 minutes with 10 μg/ml of anti-EGFR antibody (Cetuximab) that was labeled with single TCO-AF647, single TCO-AF488, or double TCO-AF488. The DOL for each antibody is indicated in the lower panel. After staining, cells were washed with 1×PBS for three times and imaged under a fluorescent microscope. After imaging, the stained cells were quenched with 50 μM H BHQ3-Tz in for 5 mins. Quenching efficiency was calculated by comparing the fluorescent intensity before and after quenching.

### Example 11—Synthesis and Evaluation of Fluorescent Double dTCO FAST Probes for Antibody Labeling

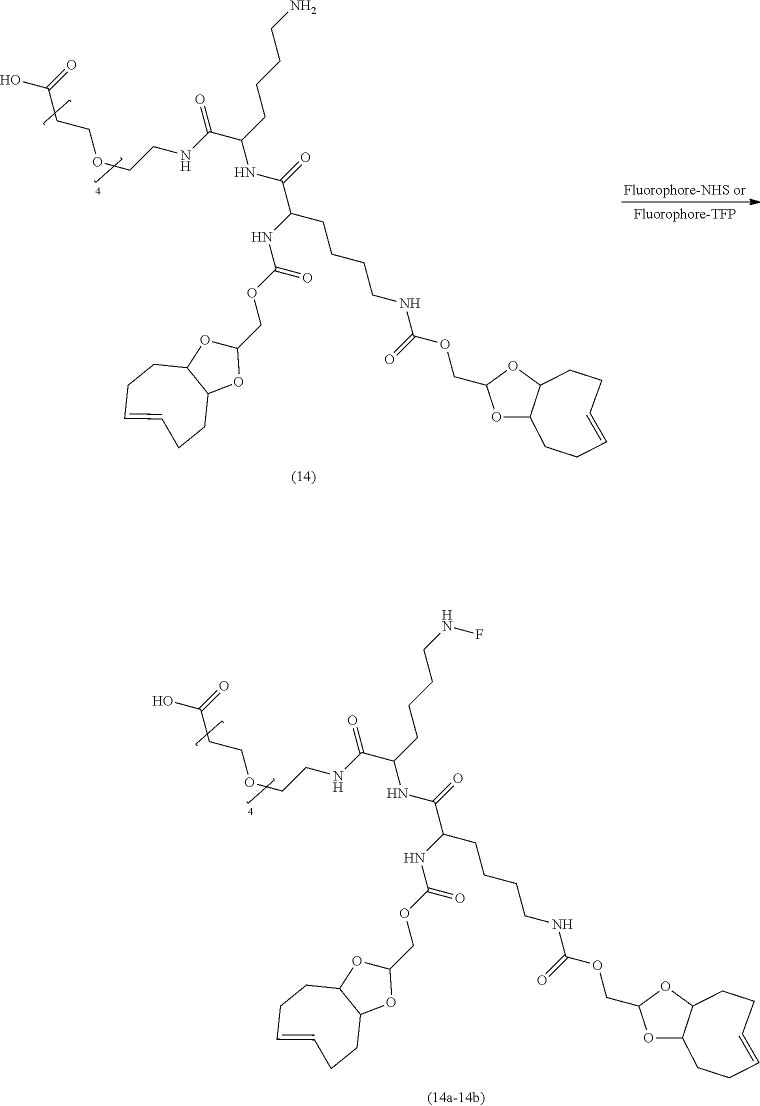

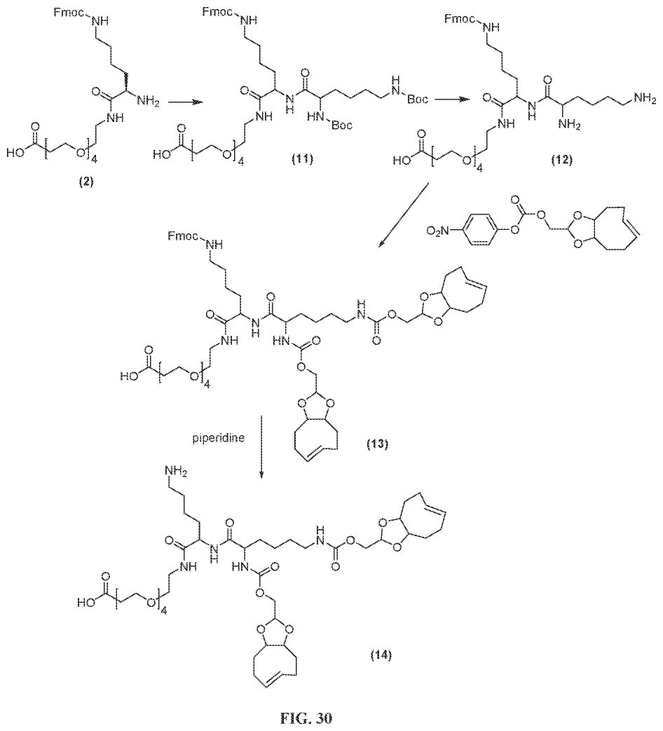

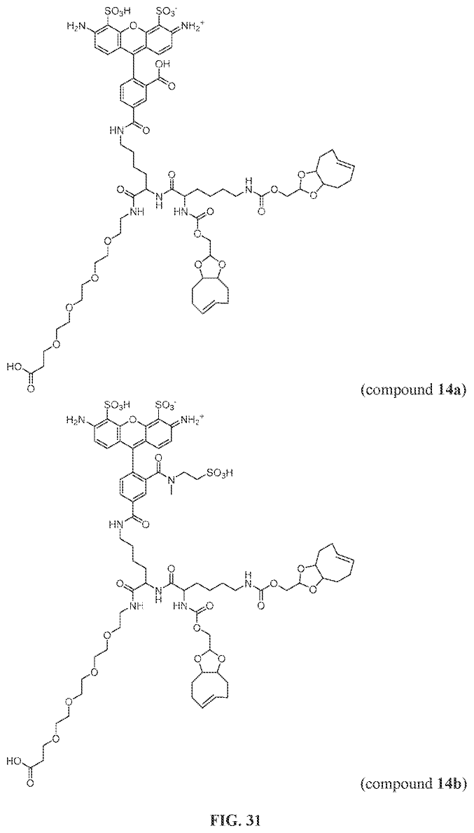

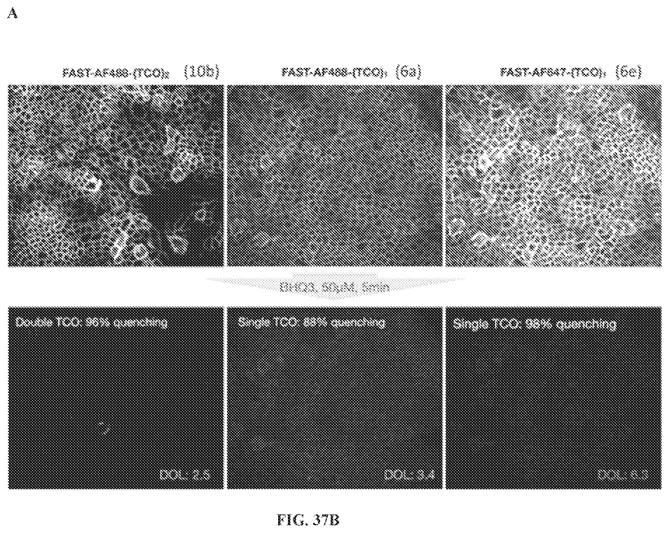

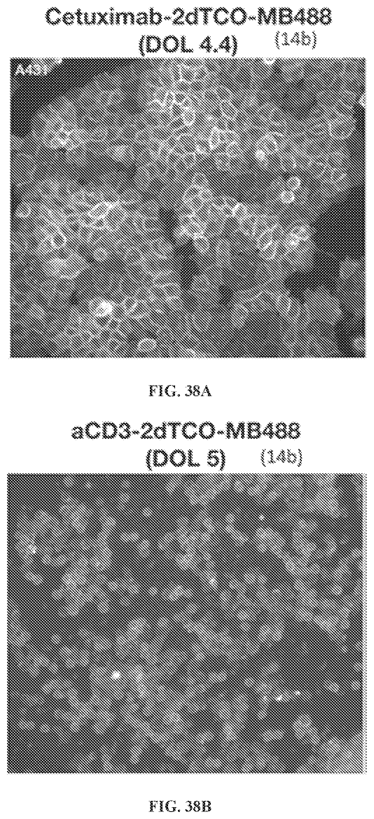

Synthesis of the key intermediate (14) is shown in FIG. 30. The intermediate (14) was prepared using methods and procedures similar to those described for intermediate (4) in Example 1. Compounds 14a-14b were prepared according to the methods and procedures similar to those described for compounds 4a-4d in Example 3. FIG. 31 contains chemical structures of FAST-AF488-dTCO₂ (14a) and FAST-MB488-dTCO₂ (14b). The results of imaging and quenching experiments using compound 14b are shown in FIGS. 38A and 38B.

Details of experiment described in FIG. 38A: A431 cells were fixed with 4% formaldehyde and then stained with 5 μg/mL Cetuximab-FAST-MB488-dTCO₂ (degree of labeling: 4.4 molecules of 14b per antibody) for 30 minutes and then washed 3× with PBS (30 seconds each) before imaging.

Details of experiment described in FIG. 38B: Anti-CD3 antibody (Biolegend 100306) was incubated with 10 equivalents of FAST-MB488-dTCO-NHS, resulting in a DOL of 5 dyes/antibody. Mouse splenocytes were incubated with anti-CD3-FASTM488dTCO₂ at 5 μg/mL for 15 minutes.

### Example 12—Synthesis and Evaluation of Fluorescent Cyclopropene FAST Probes for Antibody Labeling

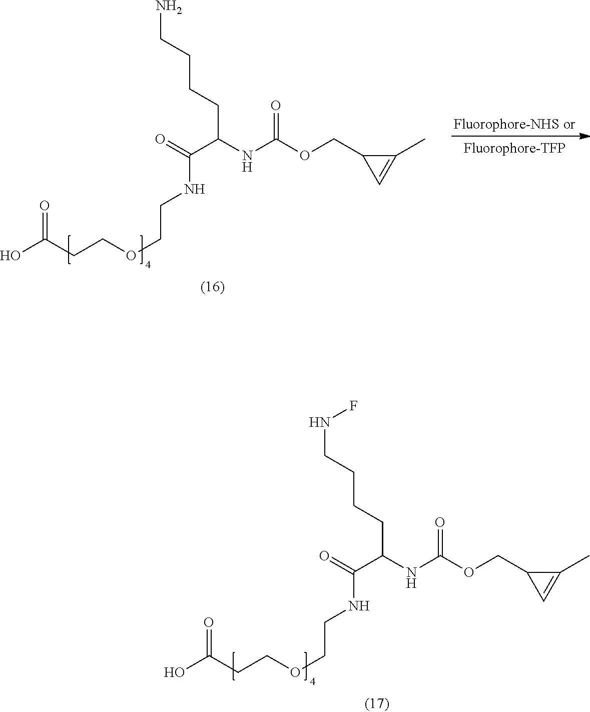

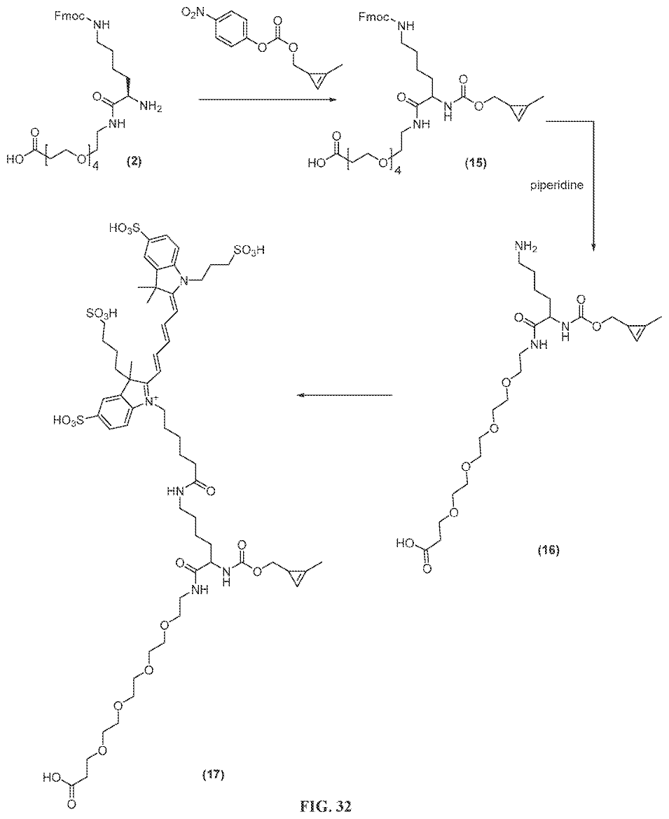

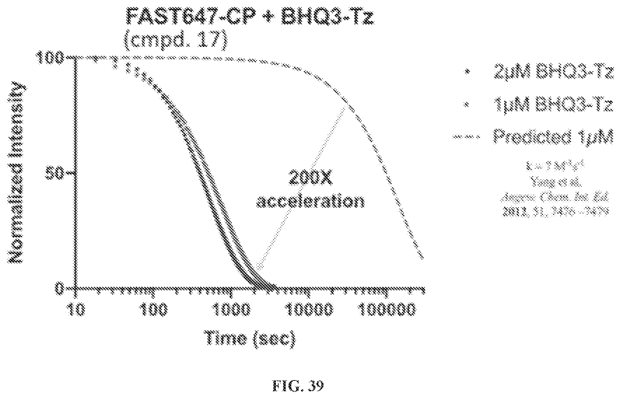

Synthesis of the key intermediate (16) is shown in FIG. 32. The intermediate (16) was prepared using methods and procedures similar to those described for intermediate (4) in Example 1. Compound 17 (FAST-AF647-cyclopropene) was prepared according to the methods and procedures similar to those described for compounds 4a-4d in Example 3. FIG. 32 contains chemical structure of FAST-AF647-cyclopropene (compound 17). The results of quenching kinetics experiments using compound 17 are shown in FIG. 39.

Details of experiment described in FIG. 39A: FAST-AF647-CP was prepared as a stock solution in DMSO (10 μM). 2 μL of this solution were added to 2 mL of PBS-bicarb (pH9) in a disposable polystyrene cuvette. The cuvette was placed in the fluorimeter with a magnetic stir bar and the baseline fluorescence intensity established. An aliquot of BHQ3-Tz (100 μM in DMSO) was then added via the fluorimeter's sample addition port to achieve the indicated concentration. Fluorescence was monitored until the signal reached a stable plateau. The predicted curve is calculated from the reported quenching kinetics, as indicated in the annotation.

### Example 13—Synthesis of Fluorescent Tz FAST Probes for Antibody Labeling

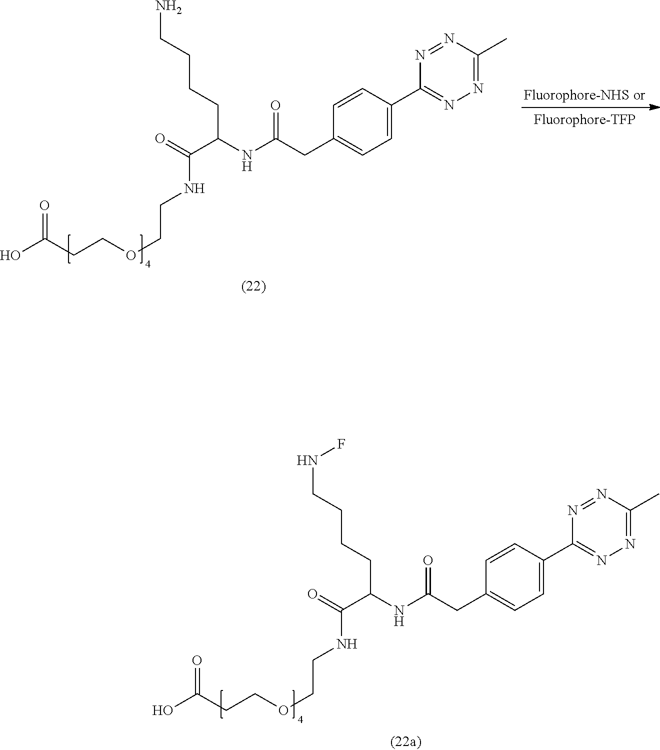

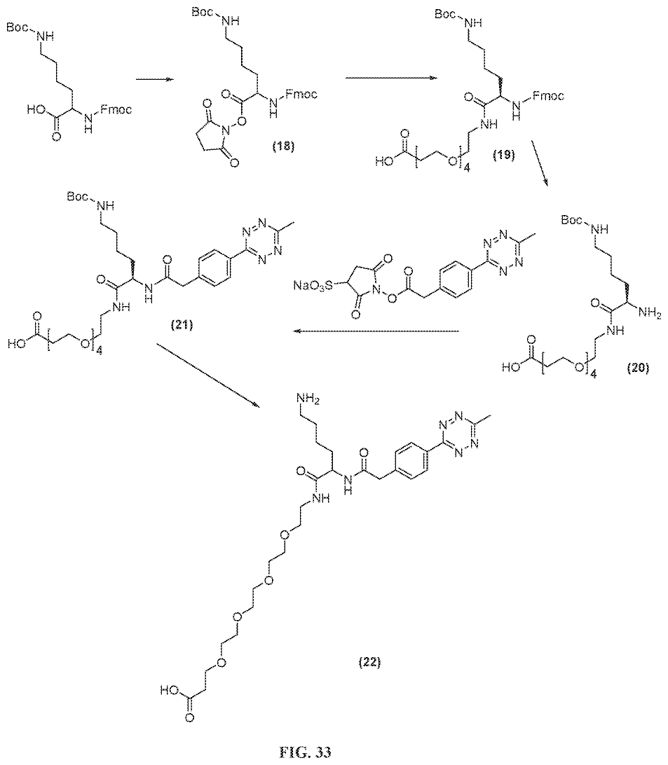

Synthesis of the key intermediate (22) is shown in FIG. 33. The intermediate (22) was prepared using methods and procedures similar to those described for intermediate (4) in Example 1. Compound 22a was prepared according to the methods and procedures similar to those described for compounds 4a-4d in Example 3.

### Example 14—Synthesis of Fluorescent FAST Probes for Antibody Labeling Via Ternary TCO Reagents

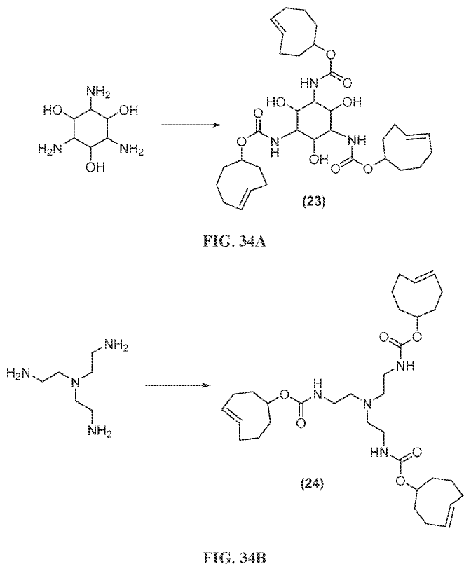

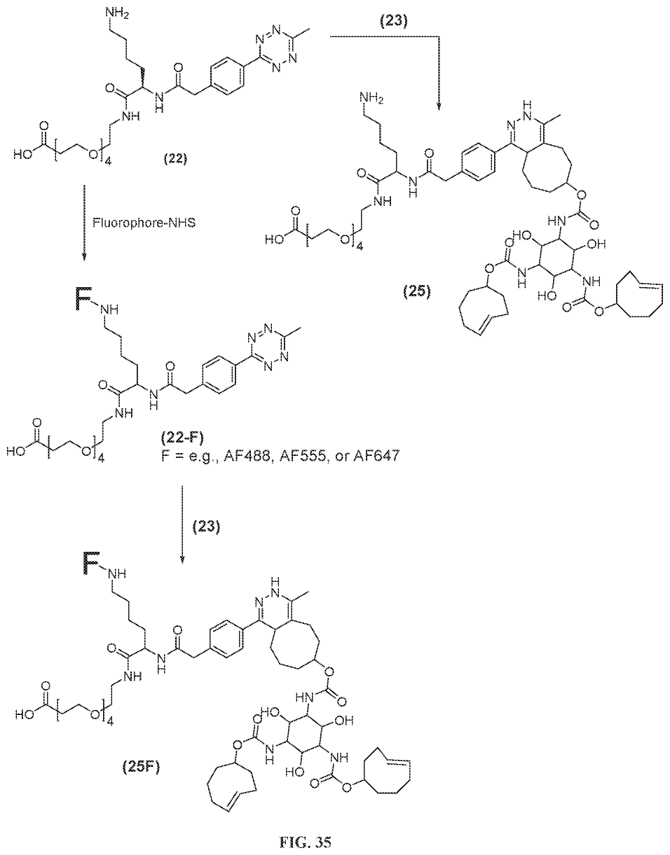

Exemplary synthesis of the ternary TCO reagents is shown in FIGS. 34A and 34B. Ternary TCO reagent (23) was prepared from triaminoinositol, while ternary TCO reagent (24) is prepared from tris(2-aminoethyl)amine. The ternary reagent (23) was coupled with compound (21) to produce double TCO intermediate (26), as shown in FIG. 35. The ternary reagent (24) was coupled with compound (21) to produce double TCO intermediate (28). The key intermediates (26) and (28) were further used to prepare fluorescent probes using reagents, procedures, and conditions similar to those used in Examples 9-13.

### Example 15—FAST Quenching of Live Cells

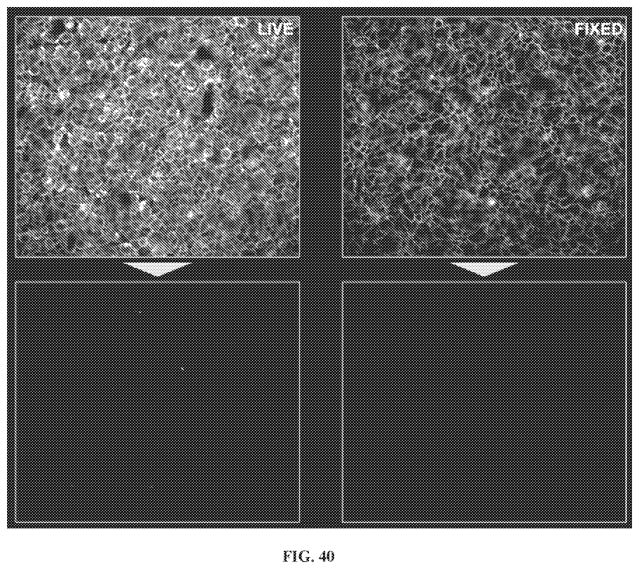

Cultured A431 cells (live and fixed) were stained with anti-EGFR antibody that was labeled with rTCO-AF647 (compound 4d) for 20 minutes. For fixed cells, cells were fixed with 4% formaldehyde for 10 minutes before staining. After staining, cells were washed with 1×PBS for three times and imaged. After imaging, cells were quenched with 20 μM H BHQ3-Tz in pH9 buffer for 5 minutes. Results of the experiment are shown in FIG. 40.

### Example 16

Head and neck squamous cell carcinoma (HNSCC) is a worldwide public health problem. The FDA has approved the use of pembrolizumab monotherapy and the combination of pembrolizumab plus chemotherapy for treatment of recurrent and/or metastatic HNSCCs. As part of the indication to administer anti-PD1 monotherapy to HNSCC patients, a combined positive score (CPS) of PD-L1 staining of greater than or equal to 1% is required in patient tumor samples since this cut off has been found to be a prognostic biomarker of response to anti-PD1 monotherapy (See Burtness et al., 2019, Lancet, 394, 1915-1928). Hence, the biomarker testing can inform treatment decisions. While PD-L1 is expressed in 85% of HNSCC patients and, only 10-20% of HNSCC patients respond to anti-PD1 therapy, indicating limitations in profiling PD-L1 expression to guide therapy. The methods described in this and other working examples allow to tailor personalized approaches through composite biomarkers for cancer immunotherapy.

As discussed in this disclosure, biopsies typically yield scant cellular materials. However, FNA is ideally suited for biomarker sampling due to the exceedingly low risk of procedural complications and the ability to obtain serial specimens to monitor response during therapy. The methods of this disclosure allow multiplexed FNA assay to measure a large number of tumor and immune cell markers through cycling. The methods and reagents described here allow rapid cell staining and destaining within only seconds, while maintaining the integrity of specimens. Given the rapidity of the cycles it is possible to image 20-40 molecular markers per cell in paucicellular FNA specimen within an hour.

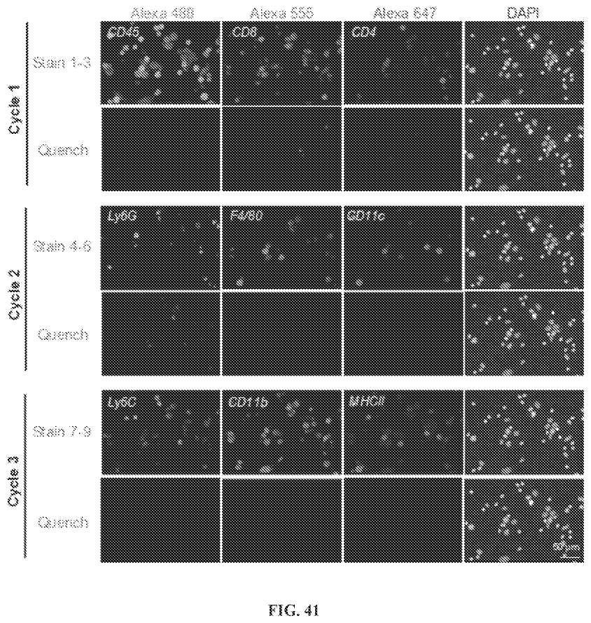

The reagents (FAST probes) described in this disclosure were conjugated to antibodies against target proteins of interest (See FIGS. 44 and 45) as described in the working examples 1-15. Imaging cycles were repeated on the collected specimens until all target proteins of interest were imaged (FIG. 41 shows an example field of view of a mouse tumor FNA sample for the first 3 cycles of FAST imaging).

TME Analysis of Mouse Tumors Using Fine Needle Aspiration

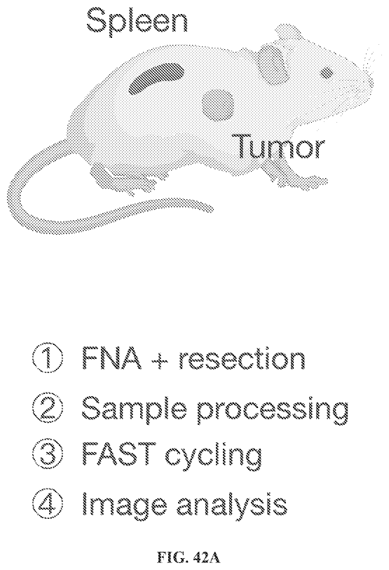

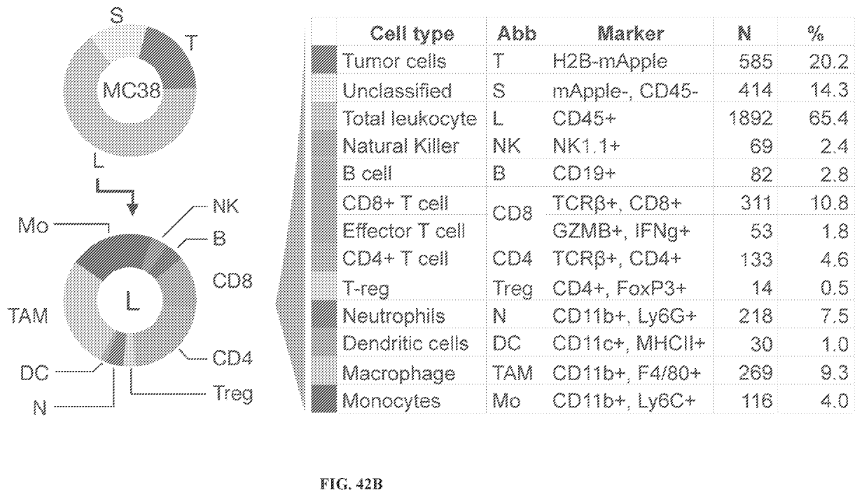

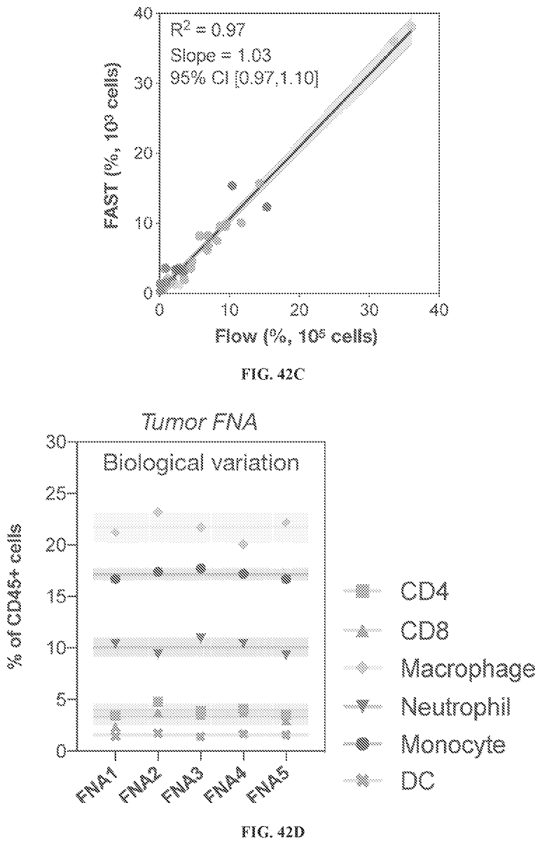

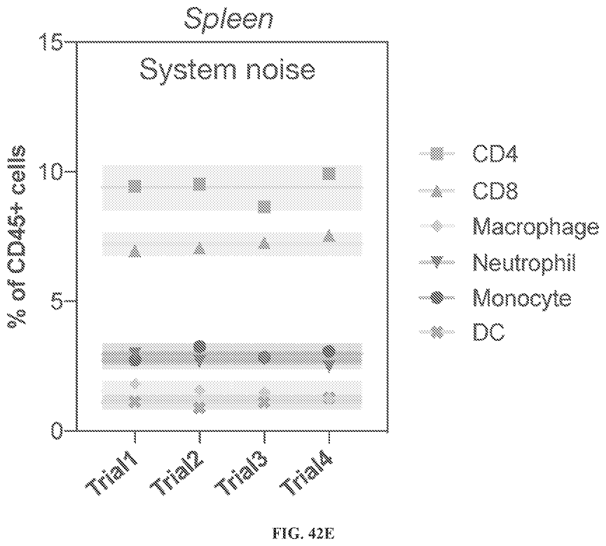

The cellular makeup of a mouse tumor microenvironment was determined. MC38 mouse colon cancer model was used in the study. When tumors had grown to about 40 mm³ in size, FNA were obtained followed by whole tumor removal for comparative flow cytometry analysis (See FIG. 42A). The tumor cells expressed H2B-mApple, which also allowed to identify three major cell populations, namely: H2B-mApple⁺ CD45⁻ tumor cells, CD45⁺ leukocytes, and H2B-mApple CD45⁻ non-immune non-tumor cells (including fibroblasts, endothelial cells). In one example, 2891 cells were analyzed by FAST-FNA, of which 1892 were CD45⁺ immune cells and 585 were mApple⁺ CD45⁻ tumor cells.

FAST-FNA was used to identify distinct cell populations among CD45⁺ cells. Tumor-infiltrating CD8⁺ T cells were assessed, including those expressing effector (Granzyme B⁺, IFN-g⁺) or immunosuppressive markers (PD-1V). Conventional CD4⁺ T cells and regulatory CD4⁺ T cells were also evaluated. B cells were also evaluated, along with various myeloid cells, including tumor-infiltrating monocytes, neutrophils, macrophages, and tumor-infiltrating dendritic cells. Each cell type was analyzed for 20-30 biomarkers.

A direct comparison of FAST-FNA and flow cytometry data from the same tumor samples revealed that FAST-FNA is equally efficient at detecting and enumerating the different populations mentioned above (R²=0.97, FIG. 42B). For example, dendritic cells (DC) and regulatory T cells (Treg) were both relatively rare within MC38 tumors, whereas monocytes, macrophages and neutrophils were more abundant. These findings indicate that fine needle aspirates traversing distinct xyz coordinates within a given tumor are reflective of the entire TME composition. Noteworthily, FNA samples were not of sufficient cellularity for primary flow cytometry analyses which requires many more cells.

Variability between different needle passes was also determined. To this end, five serial FNA samples were collected from the same tumor. These independent samples showed small variations within the 95% confidence interval of the mean (FIG. 42C). Mouse spleen homogenates were used to determine temporal variations in the FAST-FNA assay. Aliquots were analyzed on four separate days, and only small variations in abundance of the different cell types analyzed were detected (FIG. 42D), further indicating high reproducibility of the methods disclosed herein.

Anti-PD1 Immunotherapy Induces TME Changes Detectable by Serial FNA Analysis

Mice bearing MC38 tumors were treated with an anti-PD1 monoclonal antibody (mAb). Half of MC38 tumors progressively regressed in size in response to anti-PD1 (“responders”), while the remainder continued to grow over time (“non-responders”). FAST-FNA analysis was able to identify distinct phenotypes for these two operationally separated cohorts. In all animals, the CD8+ T cell fraction increased to some extent, although only the responders maintained high expression levels of granzyme B and/or IFN-γ in the CD8+ T cell population. PD-1 expression decreased over time in both responders and non-responders.

FNA Analysis of Human HNSCC

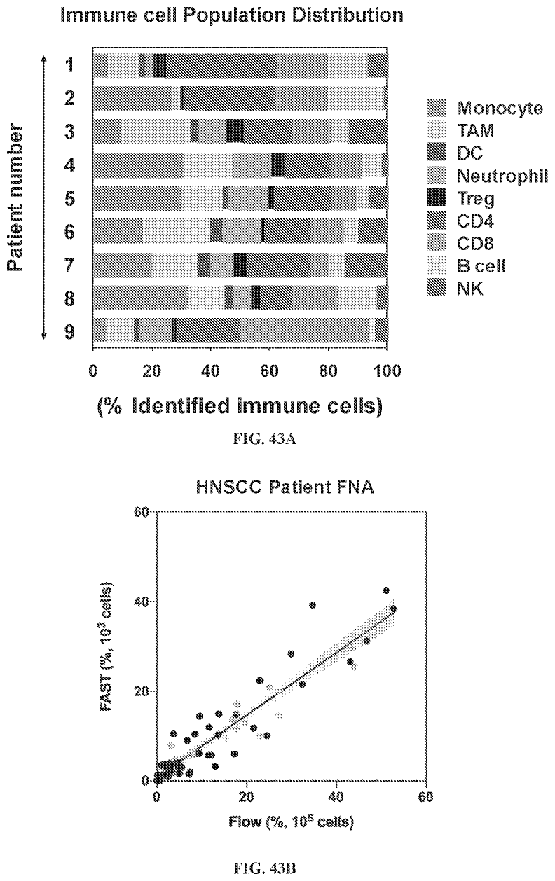

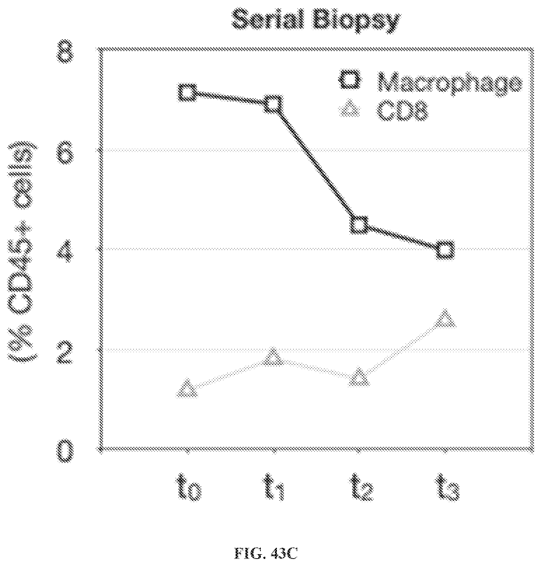

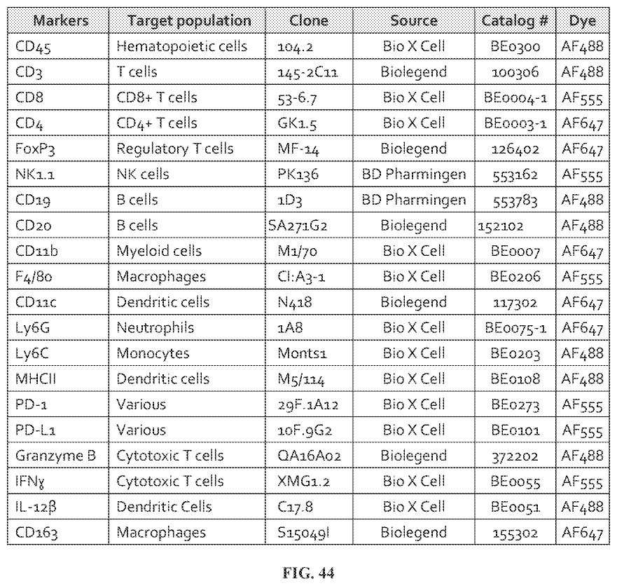

FNA samples were collected from enrolled subjects (See FIGS. 43A-43C) prior to surgical resection of the HNSCC tumor. FAST-FNA results could be directly compared to tissues samples processed for flow cytometry. A remarkable correlation coefficient of R²=0.86 was attained for the main immune cell types across 9 patients.

FAST-FNA results were used to determine the relative abundance of tumor, immune, and other cells in the 9 patients. A significant difference between HPV+ and HPV− HNSCC was noted: HPV⁺ HNSCC had a higher immune infiltration than HPV⁻ HNSCC samples (78% vs 58%, p=0.04), also there were substantially more T cells (CD4⁺ and CD8⁺ combined) in HPV⁺ HNSCC than in HPV⁻ HNSCC samples (49% vs 28%, p=0.02). These results are in line with previous studies based on immunohistochemistry and RNA-seq data from HNSCC patients in the cancer genome atlas (TCGA) dataset (See Partlovi et al., 2015, Oncoimmunology, 4, e965570; see also Chen et al., 2018, Mol Immunol, 96, 28-36). In sum, FAST-FNA is advantageously useful for non-invasively analyzing the TME in human HNSCC.

PD-L1 Profiling of the TME Landscape in HNSCC

PD-L1 expression is a predictive biomarker of response of HNSCC to anti-PD1 monotherapy. The methods and reagents of this disclosure provide a same day diagnostic method that enables treatment of this type of cancer without unnecessary delay.

In this example, for each patient's tumor, the following was determined: (i) the frequency of PD-L1 cells defined by FAST-FNA analysis, and (ii) the CPS obtained by IHC on whole tissue. The results show positive and linear correlation between the two metrics (R²=0.82).

FAST-FNA method was also used to uncover the identity of the cells expressing PD-L1. The results show that many cell types can contribute to overall PD-L1 expression within tumors. PD-L1⁺ cells include tumor cells, monocytes, macrophages, and also neutrophils, various lymphocyte populations, and stromal cells. Further, relative contribution of cell types that are PD-L1⁺ can vary greatly across patients. In some patients, PD-L1 is mainly expressed in macrophages as opposed to tumor cells. Hence, the instant methods provide not only CPS scoring (as was possible using prior methods), but also quantitatively assess PD-L1 expression, and attribute it to tumor and/or various immune cells (which the prior methods cannot achieve).

Time Course Analysis of TME in HNSCC Under Immunotherapy

The instant methods also allow capture of changes in the TME during active immunotherapy treatment. As the experimental results show, FAST-FNA can be used for comprehensive profiling of the TME (both tumor and immune cells) in HNSCC and for predicting clinical response to immunotherapy. Serial FNA samples were obtained from a HNSCC patient who received immunotherapy treatment. Specifically, the patient received anti-CD40 and anti-PD-1 treatments on weeks 0, 2, 5 and 7, and FAST-FNA samples were collected on weeks −2, 0, 2, 5 and 7.

The results show that it is feasible to obtain serial TME data during the course of a treatment. Also, the procedure was well tolerated. In this particular example, the most notable changes were the continuous decrease in macrophage and B cell fractions after immunotherapy, whereas NK cell and CD8⁺ T cell fractions increased on week 7. Marked increases in T cell response markers were also observed (Granzyme B, IFNγ, CD39, TCF1) in CD8⁺ T cells suggesting that T cell states have changed in response to the immunotherapy. Also, previous studies have shown that IFNg production by DC controls local IL-12 production, which is necessary for achieving tumor control following anti-PD1 and anti-CD40 treatment (See Garris et al., 2018, Immunity, 49, 1148-1161.e7). Notably, the expression level of IL-12 in DCs in the serial FNA samples showed sporadic increase at week 2 and 7).

Discussion of Data Presented in this Example

The experimental results show that single immune cells can be harvested and characterized via fine needle aspirates and that the numbers of cells obtained are representative of the TME, and that TME can be accurately and reproducibly measured by FNA. The HNSCC samples used in this example are not limiting: the methods of this disclosure can be applied to other malignancies where rapid answers are desirable or where repeat core biopsies are unavailable or pose a clinical risk.

The analysis showed that FAST-FNA and flow cytometry are positively correlated with a R²=0.89 in mouse samples and an R²=0.86 in human samples, when the minimum number of cells required to perform these two assays are vastly different. For flow cytometry, surgical tissue had to be dissociated into single cell suspensions so that 10⁵-10⁶ cells could be analyzed. In contradistinction, FNA were directly obtained from patients and samples typically contained about 10³ cells. Further, experimental results show that access to information about multiple biomarkers may be better at predicting outcomes than a single initial score (such as CPS score of >1% for HNSCC, as currently required by FDA for using anti-PD-1 therapy to treat this type of cancer).

### CONCLUSIONS

In sum, the working examples describe an ultra-fast, highly efficient but yet gentle quenching technology for multiplexed protein profiling in single cells (including live cells). TCO-Tz click chemistry was used for staining, quenching, and cycling of fluorescence imaging. The present disclosure provides a new method for rapid dye activation immediately prior to antibody labeling. Using the methods within the present claims, 12 immune markers were successfully stained within one hour from the same cells, which enabled profiling of immunocyte populations in the tumor microenvironment The ultra-fast quenching allows rapid repeat multi-color staining. A <10 second step is sufficient to remove >95% of the fluorescence signal from the previous cycle. The observed quenching speed is unexpectedly much faster than the predicted bimolecular TCO-Tz reaction rate; this was observed for all fluorochromes tested (FIG. 3C). Without being bound by a theory, the experimental evidence suggests that transient complexation between dye and quencher markedly accelerates the TCO-Tz click through an effect on local concentration. Irrespective of the exact mechanism, the ultrafast quenching is a highly distinct feature compared to alternative methods where at least 30 minute incubation with chemicals or water is necessary. The methods within the present claims can be practically used to image many different sample types including FNA and tissues as the process does not involve usage of any strong chemicals nor other harsh destaining conditions.

### REFERENCES

[1] R. Weissleder et al., Sci Transl Med 2016, 8, 355ps16.
[2] M. Basik, et al., Nat Rev Clin Oncol 2013, 10, 437.
[3] A. F. Sarioglu et al., Nat Methods 2015, 12, 685.
[4] R. Shah et al., N Engl J Med 2018, 379, 958.
[5] R. B. Corcoran et al., N Engl J Med 2019, 380, 501.; C. Fiala et al., N Engl J Med 2019, 380, 501.
[6] N. E. Frenk et al., JCO Precision Oncology 2017, 1, 1.
[7] R. Zilionis et al., Immunity 2019, 50, 1317.; A. C. Villani et al., Science 2017, 356.
[8] J. R. Lin et al., Nat Commun 2015, 6, 8390.; M. J. Gerdes et al., Proc Natl Acad Sci USA 2013, 110, 11982.; W. Schubert et al., Nat Biotechnol 2006, 24, 1270.
[9] A. V. Ullal et al., Sci Transl Med 2014, 6, 219ra9.; S. S. Agasti et al., J Am Chem Soc 2012, 134, 18499.
[10] R. J. Giedt et al., Nat Commun 2018, 9, 4550.
[11] S.-M. Guo et al., Nature communications 2019, 10, 1.; Y Goltsev et al., Cell 2018, 174, 968.; S. K. Saka et al., Nat Biotechnol 2019, 37, 1080.
[12] J. C. T. Carlson et al. J Am Chem Soc 2018, 140, 3603.
[13] G. Budin et al., Angewandte Chemie International Edition 2012, 51, 7752.; N. K. Devaraj et al., Accounts of chemical research 2011, 44, 816.; L. G. Meimetis et al., Angew Chem Int Ed Engl 2014, 53, 7531.; L. G. Meimetis et al., Bioconjug Chem 2016, 27, 257.; K. S. Yang et al., Angewandte Chemie International Edition 2013, 52, 10593.
[14] A. Darko et al., Chemical science 2014, 5, 3770.
[15] R. M. Versteegen et al., Angewandte Chemie International Edition 2013, 52, 14112.
[16] J. Li et al., Nat Chem Biol 2014, 10, 1003.
[17] L. D. Lavis, Annual review of biochemistry 2017, 86, 825.
[18] X. Peng et al., Analytical biochemistry 2009, 388, 220.
[19] S. A. E. Marras et al., Nucleic acids research 2002, 30, e122.
[20] V. M. Farzan et al., Analytical Methods 2016, 8, 5826.
[21] M. Braner et al., Chemical Communications 2017, 53, 545.
[22] M. K. Johansson et al., J Am Chem Soc 2002, 124, 6950.
[23] C. S. Garris et al., Immunity 2018, 49, 1148.; J. Oh et al., JCI Insight 2018, 3,
[24] F. Zhao et al., 2006 IEEE International Conference on Acoustics Speech and Signal Processing Proceedings 2, II.
[25] D. M. Mosser et al., Nat Rev Immunol 2008, 8, 958.
[26] R. M. Versteegen et al., Angewandte Chemie International Edition 2013, 52, 14112.

### NUMBERED PARAGRAPHS

In some embodiments, the invention of the present disclosure can be described by reference to the following numbered paragraphs:
1. A compound of Formula (A):

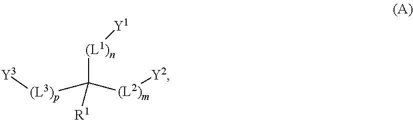

or a pharmaceutically acceptable salt thereof, wherein:
R¹ is selected from H, halo, C₁₋₆ alkyl, C₁₋₆ haloalkyl, C₁₋₆ alkoxy, and C₁₋₆ haloalkoxy;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
n is an integer from 1 to 10;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
m is an integer from 1 to 10;
each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ, —(CH₂CH(CH₃)O)ₓ, and a moiety formed by a click reaction, wherein said C₁₋₆ alkylene, C₃₋₇ cycloalkylene, C₆₋₁₀ arylene, —(OCH₂CH₂)ₓ, —(CH₂CH₂O)ₓ, —(OCH(CH₃)CH₂)ₓ, and —(CH₂CH(CH₃)O)ₓ— are each optionally substituted with 1, 2, 3, 4, or 5 substituents independently selected from OH, NH₂, C₁₋₆ alkylamino, di(C₁₋₆-alkyl)amino, C₁₋₆ haloalkyl, C₁₋₆ alkoxy, C₁₋₆ haloalkoxy, and (L⁴)ₒ-Y³;
each L⁴ is independently selected from N(R^{N1}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—, p is an integer from 1 to 20;
is an integer from 1 to 10;
each x is independently an integer from 1 to 2,000;
each R^{N} is independently selected from H, C₁₋₃ alkyl, C₁₋₃ haloalkyl, and (L⁴)ₒ-Y³;
each R^{N1} is independently selected from H, C₁₋₃ alkyl, and C₁₋₃ haloalkyl;
Y¹ is selected from NR^{c1}R^{1A}, OR², and C(═O)R³;
R^{1A} selected from H, an amine protecting group, and a residue of a fluorophore;
R² is selected from H, an alcohol protecting group, and a residue of a fluorophore;
R³ is selected from OR^{a1} and a residue of a fluorophore;
Y² is selected from C(═O)OR^{a1}, NR^{c1}R⁴, OR⁵; and a group reactive with a side chain of an amino acid of a protein;
R^{a1} is selected from H and a carboxylic acid protecting group;
R is selected from H, C₁₋₃ alkyl, and C₁₋₃ haloalkyl;
R⁴ is selected from H and an amine protecting group;
R⁵ is selected from H and an alcohol protecting group; and
Y³ is a chemical group that is reactive in a biorthogonal chemical reaction.
2. The compound of paragraph 1, wherein the compound of Formula (A) has Formula (I):

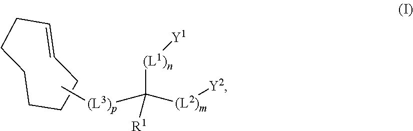

or a pharmaceutically acceptable salt thereof, wherein:
R¹ is selected from H, halo, C₁₋₆ alkyl, C₁₋₆ haloalkyl, C₁₋₆ alkoxy, and C₁₋₆ haloalkoxy;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
n is an integer from 1 to 10;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
m is an integer from 1 to 10;
each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—, p is an integer from 1 to 10;
each x is independently an integer from 1 to 2,000;
each R^{N} is independently selected from H, C₁₋₃ alkyl, and C₁₋₃ haloalkyl;
Y¹ is selected from NR^{c1}R^{1A}, OR², and C(═O)R³;
R^{1A} selected from H, an amine protecting group, and a residue of a fluorophore;
R² is selected from H, an alcohol protecting group, and a residue of a fluorophore;
R³ is selected from OR^{a1} and a residue of a fluorophore;
Y² is selected from C(═O)OR^{a1}, NR^{c1}R⁴, OR⁵; and a group reactive with a side chain of an amino acid of a protein;
R^{a1} is selected from H and a carboxylic acid protecting group;
R is selected from H, C₁₋₃ alkyl, and C₁₋₃ haloalkyl;
R⁴ is selected from H and an amine protecting group; and
R⁵ is selected from H an alcohol protecting group.
3. The compound of paragraph 1 or 2, wherein R¹ is H.
4. The compound of any one of paragraphs 1-3, wherein n is an integer from 1 to 5, and each L¹ is selected from NH, O, C(═O), C₁₋₆ alkylene, and C₆₋₁₀ arylene.
5. The compound of paragraph 4, wherein n is 1 and L¹ is C₁₋₆ alkylene.
6. The compound of any one of paragraphs 1-5, wherein m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—.
7. The compound of paragraph 6, wherein m is 4, and each L² is independently selected from NH, C(═O), C₁₋₆ alkylene, and —(OCH₂CH₂)ₓ—.
8. The compound of any one of paragraphs 1-7, wherein x is an integer from 2 to 10.
9. The compound of any one of paragraphs 1-8, wherein Y¹ is NHR^{1A}
10. The compound of paragraph 9, wherein R^{1A} is a residue of a fluorophore.
11. The compound of paragraph 9, wherein R^{1A} is an amine protecting group.
12. The compound of paragraph 9, wherein Y¹ is NH₂.
13. The compound of any one of paragraphs 1-8, wherein Y¹ is OR².
14. The compound of paragraph 13, wherein Y¹ is OH.
15. The compound of paragraph 13, wherein R² is an alcohol protecting group.
16. The compound of paragraph 13, wherein R² is a residue of a fluorophore.
17. The compound of any one of paragraphs 1-8, wherein Y¹ is C(═O)R³.
18. The compound of paragraph 17, wherein Y¹ is C(═O)OH.
19. The compound of paragraph 17, wherein R³ is OR^{a1}, and R^{a1} is a carboxylic acid protecting group.
20. The compound of paragraph 17, wherein R³ is a residue of a fluorophore.
21. The compound of any one of paragraphs 1-20, wherein Y² is C(═O)OR^{a1}.
22. The compound of paragraph 21, wherein Y² is C(═O)OH.
23. The compound of paragraph 21, wherein R^{a1} is a carboxylic acid protecting group.
24. The compound of any one of paragraphs 1-20, wherein Y² is NHR⁴.
25. The compound of paragraph 24, wherein Y² is NH₂.
26. The compound of paragraph 24, wherein R⁴ is an amine-protecting group.
27. The compound of any one of paragraphs 1-20, wherein Y² is OR⁵.
28. The compound of paragraph 27, wherein Y² is OH.
29. The compound of paragraph 27, wherein R⁵ is an alcohol-protecting group.
30. The compound of any one of paragraphs 1-20, wherein Y² is a group reactive with a side chain of an amino acid of a protein.
31. The compound of paragraph 30, wherein the group reactive with a side chain of an amino acid of a protein is an activated ester group.
32. The compound of any one of paragraphs 1-31, wherein:
   Y¹ is NHR^{1A}; and
   Y² is selected from C(═O)OR^{a1} and a group reactive with a side chain of an amino acid of a protein.
33. The compound of any one of paragraphs 1-31, wherein:
   Y¹ is NH₂; and
   Y² is C(═O)OH.
34. The compound of any one of paragraphs 1-31, wherein:
   Y¹ is NHR^{1A}.
   R^{1A} is an amine protecting group; and
   Y² is C(═O)OH.
35. The compound of any one of paragraphs 1-31, wherein:
   Y¹ is NHR^{1A}.
   R^{IA} is a residue of a fluorophore; and
   Y² is C(═O)OH.
36. The compound of any one of paragraphs 1-31, wherein:
   Y¹ is NHR^{1A}.
   R^{1A} is a residue of a fluorophore;
   Y² is C(═O)OR^{a1}; and
   R^{a1} is a carboxylic acid protecting group.
37. The compound of any one of paragraphs 1-31, wherein:
   Y¹ is NHR^{1A}.
   R^{1A} is a residue of a fluorophore; and
   Y² is a group reactive with a side chain of an amino acid of a protein.
38. The compound of any one of paragraphs 1-37, wherein p is an integer from 1 to 15.
39. The compound of any one of paragraphs 1-37, wherein p is an integer from 1 to 10.
40. The compound of any one of paragraphs 1-37, wherein p is an integer from 1 to 7.
41. The compound of any one of paragraphs 1 and 3-40, wherein each L³ is independently selected from N(R^{N}), O, C(═O), C₁₋₆ alkylene, C₆₋₁₀ arylene, C₃₋₇ cycloalkylene, and a moiety formed by a click reaction, wherein said C₁₋₆ alkylene, C₃₋₇ cycloalkylene, and C₆₋₁₀ arylene are each optionally substituted with 1, 2, 3, or 4 substituents independently selected from OH and (L⁴)ₒ-Y³.
42 The compound of any one of paragraphs 1 and 3-41, wherein the moiety formed by a click reaction is selected from:

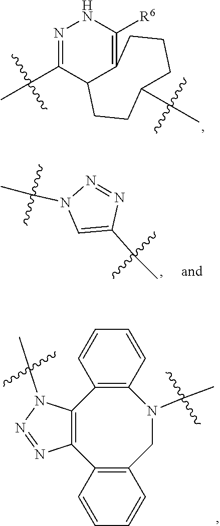

wherein R⁶ is selected from H and C₁₋₆ alkyl.
43. The compound of any one of paragraphs 1 and 3-42, wherein at least one L³ is N, and R^{N} is (L⁴)ₒ-Y³.
44. The compound of any one of paragraphs 1 and 3-43, wherein o is an integer from 1 to 7.
45. The compound of any one of paragraphs 1 and 3-43, wherein o is an integer from 1 to 5.
46. The compound of any one of paragraphs 1 and 3-45, wherein each L⁴ is independently selected from NH, O, C(═O), and C₁₋₆ alkylene.
47. The compound of any one of paragraphs 1-37, wherein:
   R¹ is H;
   n is an integer from 1 to 5, and each L¹ is selected from NH, O, C(═O), C₁₋₆ alkylene, and C₆₋₁₀ arylene;
   m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—;
   x is an integer from 2 to 10;
   p is an integer from 1 to 15;
   each L³ is independently selected from N(R^{N}), O, C(═O), C₁₋₆ alkylene, C₆₋₁₀ arylene, C₃₋₇ cycloalkylene, and a moiety formed by a click reaction, wherein said C₁₋₆ alkylene, C₃₋₇ cycloalkylene, and C₆₋₁₀ arylene are each optionally substituted with 1, 2, 3, or 4 substituents independently selected from OH and (L⁴)ₒ-Y³;
   each R^{N} is independently selected from H and (L⁴)ₒ-Y³;
   is an integer from 1 to 5; and
   each L⁴ is independently selected from NH, O, C(═O), and C₁₋₆ alkylene.
48. The compound of any one of paragraphs 1-37, wherein each L³ is independently selected from NH, O, C(═O), C₁₋₆ alkylene, C₆₋₁₀ arylene, and —(OCH₂CH₂)ₓ—.
49. The compound of any one of paragraphs 1-37 wherein p is an integer from 1 to 7, and each L³ is independently selected from NH, O, C(═O), C₁₋₆ alkylene, C₆₋₁₀ arylene, and —(OCH₂CH₂)ₓ—.
50. The compound of any one of paragraphs 1-37, wherein p is 3, and each L³ is independently selected from NH, O, and C(═O).
51. The compound of any one of paragraphs 1-37, wherein: R¹ is H;
   n is an integer from 1 to 5, and each L¹ is selected from NH, O, C(═O), C₁₋₆ alkylene, and C₆₋₁₀ arylene;
   m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—;
   x is an integer from 2 to 10; and
   p is an integer from 1 to 7, and each L³ is independently selected from NH, O, C(═O), C₁₋₆ alkylene, C₆₋₁₀ arylene, and —(OCH₂CH₂)ₓ—.
52. The compound of any one of paragraphs 1-37, wherein:
   R¹ is H;
   n is 1 and L¹ is C₁₋₆ alkylene;
   m is 4, and each L² is independently selected from NH, C(═O), C₁₋₆ alkylene, and —(OCH₂CH₂)ₓ—;
   p is 3, and each L³ is independently selected from NH, O, and C(═O); and
   x is an integer from 2 to 10.
53. The compound of any one of paragraphs 1 and 3-47, wherein Y³ comprises a chemical group selected from an azide (—N₃), an aliphatic alkyne (—C≡CH), a cyclooctyne, a cyclooctene, a cyclohexene, a nitrone, an isocyanide, a cyclopropene, a norborene, a diphenylphosphine, nitrile imine, a tetrazole, a nitrile oxide, and a tetrazine.
54. The compound of paragraph 53, wherein Y³ comprises a chemical group selected from any one of the following groups:

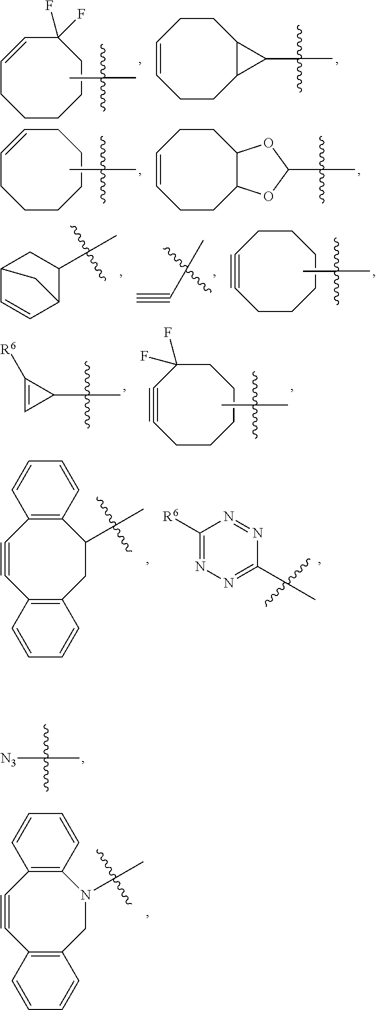

wherein R⁶ is selected from H and C₁₋₆ alkyl.
55. The compound of paragraph 54, wherein Y³ comprises a chemical group selected from:

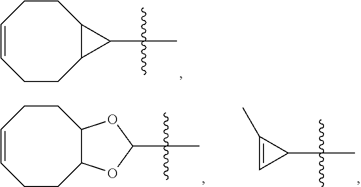

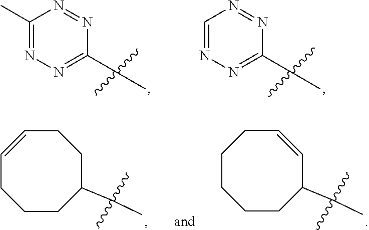

56. The compound of paragraph 1, wherein the compound of Formula (A) has formula:

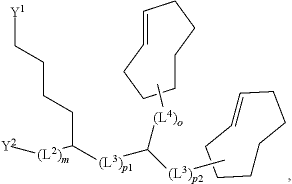

or a pharmaceutically acceptable salt thereof, wherein
the sum of p1 and p2 is less than p by at least 1.
57. The compound of paragraph 1, wherein the compound of Formula (A) has formula:

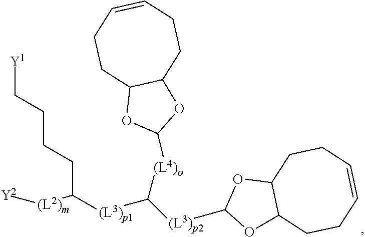

or a pharmaceutically acceptable salt thereof, wherein
the sum of p1 and p2 is less than p by at least 1.
58. The compound of paragraph 1, wherein the compound of Formula (A) has formula:

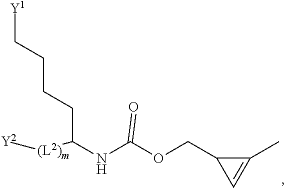

or a pharmaceutically acceptable salt thereof.
59. The compound of paragraph 1, wherein the compound of Formula (A) has formula:

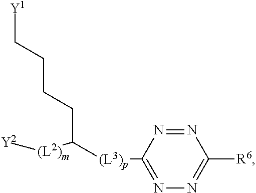

or a pharmaceutically acceptable salt thereof,
wherein R⁶ is selected from H and C₁₋₆ alkyl.
60. The compound of paragraph 1, wherein the compound of Formula (A) has formula:

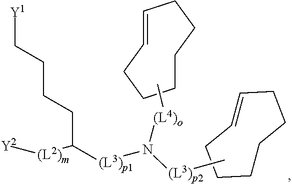

or a pharmaceutically acceptable salt thereof, wherein
the sum of p1 and p2 is less than p by at least 1.
61. The compound of paragraph 2, wherein the compound of Formula (I) has formula:

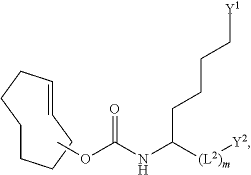

or a pharmaceutically acceptable salt thereof.
62. The compound of paragraph 2, wherein the compound of Formula (I) has formula:

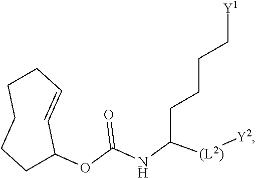

or a pharmaceutically acceptable salt thereof.
63. The compound of paragraph 2, wherein the compound of Formula (I) has formula:

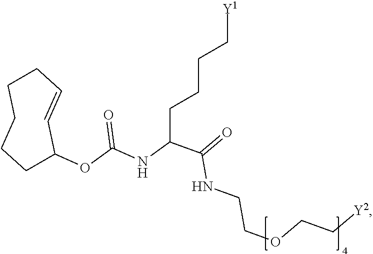

or a pharmaceutically acceptable salt thereof.
64. The compound of paragraph 1, wherein the compound of Formula (A) is selected from any one of the compounds depicted in FIGS. 1A, 15, 16A, 16B, 22A, 22B, 23-33, 35, and 36, and described in the examples 1, 3, 9, 10, 11, 12, and 13, or a pharmaceutically acceptable salt thereof.
65. The compound of paragraph 2, wherein the compound of Formula (I) is selected from any one of the compounds depicted in FIGS. 16A, 16B, 22A, and 22B, or a pharmaceutically acceptable salt thereof.
66. A protein conjugate of Formula (B):

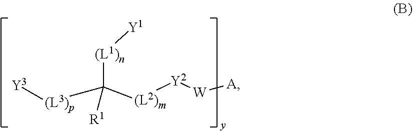

or a pharmaceutically acceptable salt thereof, wherein:
A is a residue of a protein;
y is an integer from 1 to 10;
R¹ is selected from H, halo, C₁₋₆ alkyl, C₁₋₆ haloalkyl, C₁₋₆ alkoxy, and C₁₋₆ haloalkoxy;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
n is an integer from 1 to 10;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
m is an integer from 1 to 10;
each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ, —(CH₂CH(CH₃)O)ₓ, and a moiety formed by a click reaction, wherein said C₁₋₆ alkylene, C₃₋₇ cycloalkylene, C₆₋₁₀ arylene, —(OCH₂CH₂)ₓ, —(CH₂CH₂O)ₓ, —(OCH(CH₃)CH₂)ₓ, and —(CH₂CH(CH₃)O)ₓ— are each optionally substituted with 1, 2, 3, 4, or 5 substituents independently selected from OH, NH₂, C₁₋₆ alkylamino, di(C₁₋₆-alkyl)amino, C₁₋₆ haloalkyl, C₁₋₆ alkoxy, C₁₋₆ haloalkoxy, and (L⁴)ₒ-Y³;
each L⁴ is independently selected from N(R^{N1}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—, p is an integer from 1 to 20;
is an integer from 1 to 10;
each x is independently an integer from 1 to 2,000;
each R^{N} is independently selected from H, C₁₋₃ alkyl, C₁₋₃ haloalkyl, and (L⁴)ₒ-Y³;
each R^{N1} is independently selected from H, C₁₋₃ alkyl, and C₁₋₃ haloalkyl;
Y¹ is selected from NR^{c1}R^{1A}, OR², and C(═O)R³;
R^{1A}, R², and R³ are each independently a residue of a fluorophore;
R^{c1} is selected from H, C₁₋₃ alkyl, and C₁₋₃ haloalkyl;
each W is selected from:
   (i) O of a side chain of serine, threonine, or tyrosine of the protein A;
   (ii) S of a side chain of cysteine of the protein A;
   (iii) NH of a side chain of lysine of the protein A; and
   (iv) C(═O) of a side chain of aspartic acid or glutamic acid of the protein A;
Y² is a residue of a group which, prior to conjugation with the protein A, was a group reactive with a side chain of an amino acid of the protein A; and
Y³ is a chemical group that is reactive in a biorthogonal chemical reaction.
67. The protein conjugate of paragraph 66, wherein the compound of Formula (B) has Formula (II):

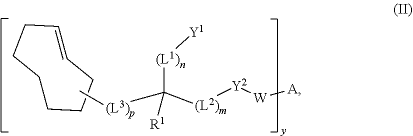

or a pharmaceutically acceptable salt thereof, wherein:
A is a residue of a protein;
y is an integer from 1 to 10;
R¹ is selected from H, halo, C₁₋₆ alkyl, C₁₋₆ haloalkyl, C₁₋₆ alkoxy, and C₁₋₆ haloalkoxy;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
n is an integer from 1 to 10;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
m is an integer from 1 to 10;
each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—, p is an integer from 1 to 10;
each x is independently an integer from 1 to 2,000;
each R^{N} is independently selected from H, C₁₋₃ alkyl, and C₁₋₃ haloalkyl;
Y¹ is selected from NR^{c1}R^{1A}, OR², and C(═O)R³;
R^{1A}, R², and R³ are each independently a residue of a fluorophore;
R^{c1} is selected from H, C₁₋₃ alkyl, and C₁₋₃ haloalkyl;
each W is selected from:
   (i) O of a side chain of serine, threonine, or tyrosine of the protein A;
   (ii) S of a side chain of cysteine of the protein A;
   (iii) NH of a side chain of lysine of the protein A; and
   (iv) C(═O) of a side chain of aspartic acid or glutamic acid of the protein A;
Y² is a residue of a group which, prior to conjugation with the protein A, was a group reactive with a side chain of an amino acid of the protein A.
68. The conjugate of paragraph 66 or 67, wherein the protein is selected from an antibody, an antibody fragment, an engineered antibody, a peptide, and an aptamer.
69. The conjugate of any one of paragraphs 66-68, wherein the antibody is specific to an antigen which is a biomarker of a disease or condition.
70. The conjugate of any one of paragraphs 66-69, wherein the disease or condition is cancer.
71. The conjugate of any one of paragraphs 66-70, wherein y is an integer from 4 to 6.
72. The conjugate of any one of paragraphs 66-71, wherein n is an integer from 1 to 5, and each L¹ is selected from NH, O, C(═O), C₁₋₆ alkylene, and C₆₋₁₀ arylene.
73. The conjugate of paragraph 72, wherein L¹ is C₁₋₆ alkylene.
74. The conjugate of any one of paragraphs 66-73, wherein m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—.
75. The conjugate of paragraph 74, wherein m is 4, and each L² is independently selected from NH, C(═O), C₁₋₆ alkylene, and —(OCH₂CH₂)ₓ—.
76. The conjugate of any one of paragraphs 66 and 68-75, wherein p is an integer from 1 to 15.
77. The conjugate of any one of paragraphs 66 and 68-75, wherein each L³ is independently selected from N(R^{N}), O, C(═O), C₁₋₆ alkylene, C₆₋₁₀ arylene, C₃₋₇ cycloalkylene, and a moiety formed by a click reaction, wherein said C₁₋₆ alkylene, C₃₋₇ cycloalkylene, and C₆₋₁₀ arylene are each optionally substituted with 1, 2, 3, or 4 substituents independently selected from OH and (L⁴)ₒ-Y³.
78. The conjugate of any one of paragraphs 66 and 68-77, wherein o is an integer from 1 to 5.
79. The conjugate of any one of paragraphs 65 and 67-77, wherein each L⁴ is independently selected from NH, O, C(═O), and C₁₋₆ alkylene.
80. The conjugate of paragraph 66, wherein:
   R¹ is H;
   n is an integer from 1 to 5, and each L¹ is selected from NH, O, C(═O), C₁₋₆ alkylene, and C₆₋₁₀ arylene;
   m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—;
   x is an integer from 2 to 10;
   p is an integer from 1 to 15;
   each L³ is independently selected from N(R^{N}), O, C(═O), C₁₋₆ alkylene, C₆₋₁₀ arylene, C₃₋₇ cycloalkylene, and a moiety formed by a click reaction, wherein said C₁₋₆ alkylene, C₃₋₇ cycloalkylene, and C₆₋₁₀ arylene are each optionally substituted with 1, 2, 3, or 4 substituents independently selected from OH and (L⁴)ₒ-Y³;
   each R^{N} is independently selected from H and (L⁴)ₒ-Y³;
   is an integer from 1 to 5; and
   each L⁴ is independently selected from NH, O, C(═O), and C₁₋₆ alkylene.
81. The conjugate of any one of paragraphs 66-75, wherein p is an integer from 1 to 7, and each L³ is independently selected from NH, O, C(═O), C₁₋₆ alkylene, C₆₋₁₀ arylene, and —(OCH₂CH₂)ₓ—.
82. The conjugate of paragraph 81, wherein p is 3, and each L³ is independently selected from NH, O, and C(═O).
83. The conjugate of any one of paragraphs 66-82, wherein x is an integer from 2 to 10.
84. The conjugate of paragraph 66, wherein:
   R¹ is H;
   n is an integer from 1 to 5, and each L¹ is selected from NH, O, C(═O), C₁₋₆ alkylene, and C₆₋₁₀ arylene;
   m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—;
   p is an integer from 1 to 7, and each L³ is independently selected from NH, O, C(═O), C₁₋₆ alkylene, C₆₋₁₀ arylene, and —(OCH₂CH₂)ₓ—; and
   x is an integer from 2 to 10.
85. The conjugate of paragraph 66, wherein:
   R¹ is H;
   n is 1 and L¹ is C₁₋₆ alkylene;
   m is 4, and each L² is independently selected from NH, C(═O), C₁₋₆ alkylene, and —(OCH₂CH₂)ₓ—;
   p is 3, and each L³ is independently selected from NH, O, and C(═O); and
   x is an integer from 2 to 10.
86. The conjugate of any one of paragraphs 66 and 68-80, wherein Y³ comprises a chemical group selected from an azide (—N₃), an aliphatic alkyne (—C≡CH), a cyclooctyne, a cyclooctene, a cyclohexene, a nitrone, an isocyanide, a cyclopropene, a norborene, a diphenylphosphine, nitrile imine, a tetrazole, a nitrile oxide, and a tetrazine.
87. The conjugate of paragraph 86, wherein Y³ comprises a chemical group selected from any one of the following groups:

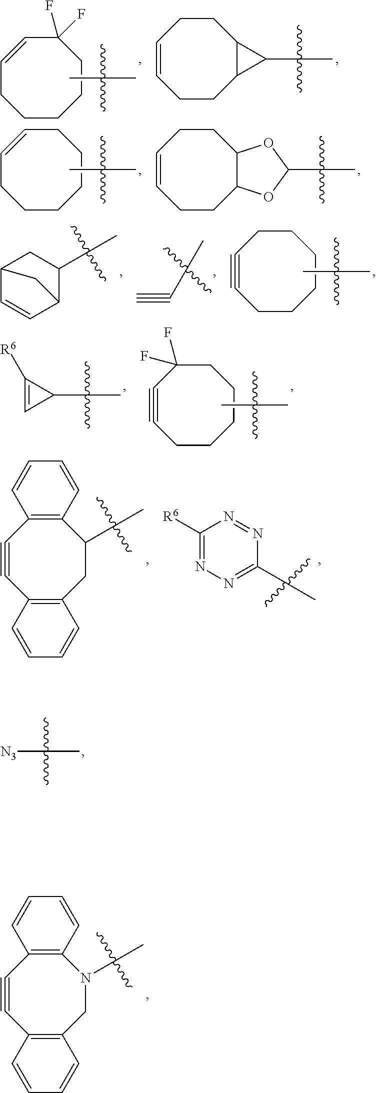

wherein R⁶ is selected from H and C₁₋₆ alkyl.
88. The conjugate of paragraph 87, wherein Y³ comprises a chemical group selected from:

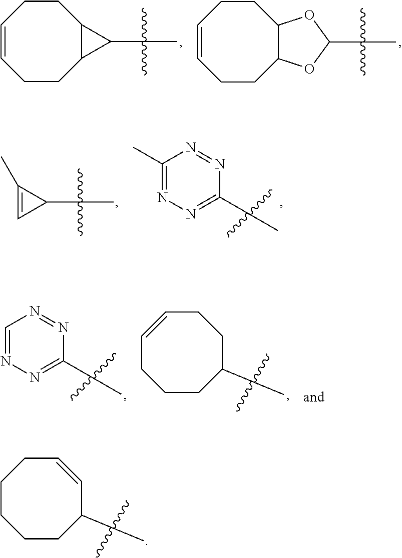

89. The conjugate of paragraph 66, wherein the conjugate of Formula (B) has formula:

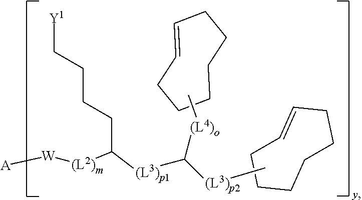

or a pharmaceutically acceptable salt thereof, wherein
the sum of p1 and p2 is less than p by at least 1.
90. The conjugate of paragraph 66, wherein the conjugate of Formula (B) has formula:

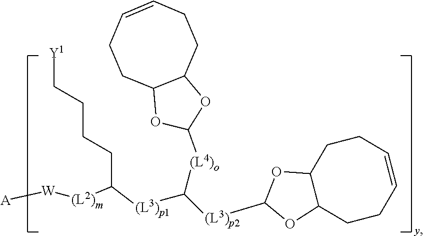

or a pharmaceutically acceptable salt thereof, wherein
the sum of p1 and p2 is less than p by at least 1.
91. The conjugate of paragraph 66, wherein the conjugate of Formula (B) has formula:

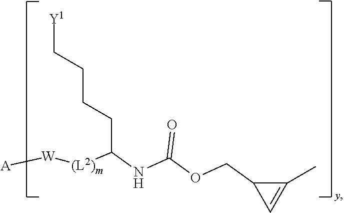

or a pharmaceutically acceptable salt thereof.
92. The conjugate of paragraph 66, wherein the conjugate of Formula (B) has formula:

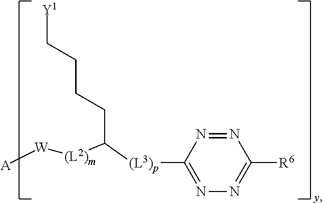

or a pharmaceutically acceptable salt thereof,
wherein R⁶ is selected from H and C₁₋₆ alkyl.
93. The conjugate of paragraph 66, wherein the conjugate of Formula (B) has formula:

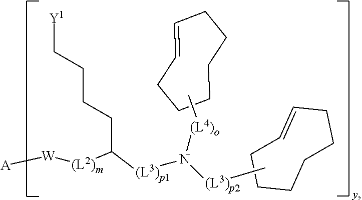

or a pharmaceutically acceptable salt thereof, wherein
the sum of p1 and p2 is less than p by at least 1.
94. The conjugate of paragraph 67, wherein the Formula (II) has formula:

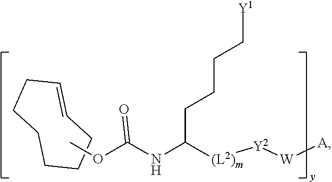

or a pharmaceutically acceptable salt thereof.
95. The conjugate of paragraph 67, wherein the Formula (II) has formula:

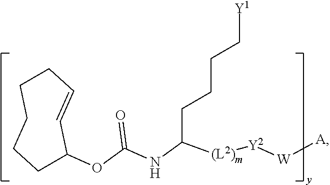

or a pharmaceutically acceptable salt thereof.
96. The conjugate of paragraph 67, wherein the Formula (II) has formula:

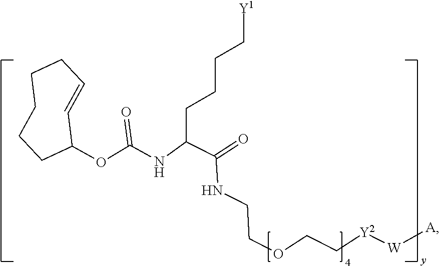

or a pharmaceutically acceptable salt thereof.
97. The conjugate of any one of paragraphs 66-96, wherein Y¹ is NHR^{1A}
98. The conjugate of any one of paragraphs 66-96, wherein Y¹ is OR².
99. The conjugate of any one of paragraphs 66-96, wherein Y¹ is C(═O)R³.
100. The conjugate of any one of paragraphs 66-96, wherein each Y² is C(═O) and each W is NH of a side chain of lysine of the protein A.
101. A composition comprising the conjugate of any one of paragraphs 66-100, or a pharmaceutically acceptable salt thereof, and an inert carrier.
102. The composition of paragraph 101, which is an aqueous solution.
103. A method of examining a cell or a component of a cell, the method comprising:
   (i) contacting the cell with a conjugate of any one of paragraphs 66-100 comprising the residue of the fluorophore, or a pharmaceutically acceptable salt thereof, or a pharmaceutical composition of any one of paragraphs 101-102;
   (ii) imaging the cell with an imaging technique; and
   (iii) after (ii), contacting the cell with a compound of Formula (C):

Y⁴-(L⁴)ₐ-Q (C),
or a pharmaceutically acceptable salt thereof, wherein:
R⁶ is selected from H, C₁₋₆ alkyl, and C₁₋₆ haloalkyl;
each L⁴ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)_{y}—, —(CH₂CH₂O)_{y}—, —(OCH(CH₃)CH₂)_{y}—, and —(CH₂CH(CH₃)O)_{y}—,
a is an integer from 1 to 10;
each R^{N} is selected from H and C₁₋₃ alkyl;
each y is an integer from 1 to 2,000;
Q is a residue of a quencher; and
Y⁴ is a chemical group that is reactive in a biorthogonal chemical reaction, wherein the contacting of step (iii) results in decrease of the fluorescence of the fluorophore in the conjugate of any one of paragraphs 66-100, or a pharmaceutically acceptable salt thereof.
104. The method of paragraph 103, comprising:
   (i) contacting the cell with a conjugate of any one of paragraphs 66-100 comprising the residue of the fluorophore, or a pharmaceutically acceptable salt thereof,
   (ii) imaging the cell with an imaging technique; and
   (iii) after (ii), contacting the cell with a compound of Formula (III):

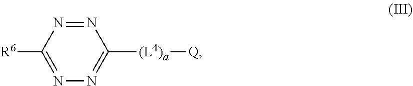

or a pharmaceutically acceptable salt thereof, wherein:
R⁶ is selected from H, C₁₋₆ alkyl, and C₁₋₆ haloalkyl;
each L⁴ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)_{y}—, —(CH₂CH₂O)_{y}—, —(OCH(CH₃)CH₂)_{y}—, and —(CH₂CH(CH₃)O)_{y}—,
a is an integer from 1 to 10;
each R^{N} is selected from H and C₁₋₃ alkyl;
each y is an integer from 1 to 2,000; and
Q is a residue of a quencher;
wherein the contacting of step (iii) results in decrease of the fluorescence of the fluorophore in the conjugate of any one of paragraphs 66-100, or a pharmaceutically acceptable salt thereof.
105. The method of paragraph 103 or 104, wherein the imaging technique is a fluorescence imaging.
106. The method of any one of paragraphs 103-105, wherein a is an integer from 1 to 7, and each L⁴ is independently selected from NH, C(═O), C₁₋₆ alkylene, C₆₋₁₀ arylene, and —(CH₂CH₂O)_{y}—.
107. The method of any one of paragraphs 103-106, wherein each y is an integer from 1 to 10.
108. The method of any one of paragraphs 103-107, wherein Y⁴ comprises a chemical group selected from an azide (—N₃), an aliphatic alkyne (—C≡CH), a cyclooctyne, a cyclooctene, a cyclohexene, a nitrone, an isocyanide, a cyclopropene, a norborene, a diphenylphosphine, nitrile imine, a tetrazole, a nitrile oxide, and a tetrazine.
109. The compound of paragraph 108, wherein Y⁴ comprises a chemical group selected from any one of the following groups:

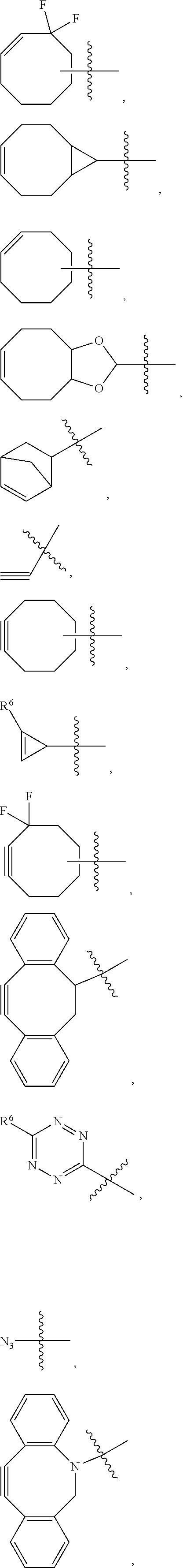

wherein R⁶ is selected from H and C₁₋₆ alkyl.
110. The method of paragraph 109, wherein Y⁴ comprises a chemical group selected from:

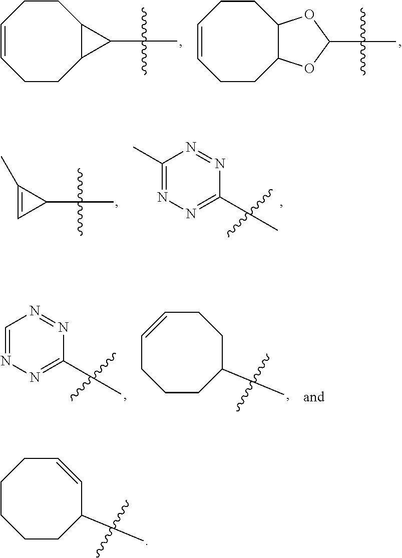

111. The method of any one of paragraphs 104-109, wherein R⁶ is H.
112. The method of paragraph 103, wherein the compound of Formula (C) has formula:

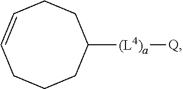

or a pharmaceutically acceptable salt thereof.
113. The method of paragraph 103, wherein the compound of Formula (C) has formula:

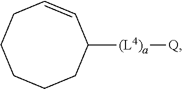

or a pharmaceutically acceptable salt thereof.
114. The method of paragraph 103, wherein the compound of Formula (C) has formula:

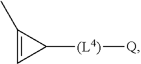

or a pharmaceutically acceptable salt thereof.
115. The method of paragraph 103, wherein the compound of Formula (C) has formula:

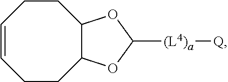

or a pharmaceutically acceptable salt thereof.
116. The method of paragraph 104, wherein the compound of Formula (III) has formula:

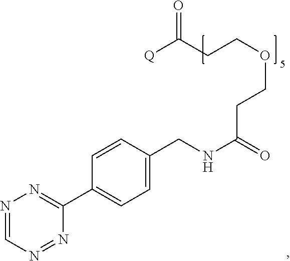

or a pharmaceutically acceptable salt thereof.
117. The method of any one of paragraphs 103 and 105-115, wherein Y⁴ in formula (C) is complementary to Y³ in the conjugate of any one of paragraphs 66-100, or a pharmaceutically acceptable salt thereof.
118. The method of paragraph 117, wherein Y⁴ comprises an azide (—N₃) and Y³ comprises an aliphatic alkyne (—C≡CH) or a cyclooctyne.
119. The method of paragraph 117, wherein Y⁴ comprises an aliphatic alkyne (—C≡CH) or a cyclooctyne and Y³ comprises an azide (—N₃).
120. The method of paragraph 117, wherein Y⁴ comprises a cyclooctene or a cyclopropene and Y³ comprises a tetrazine.
121. The method of paragraph 117, wherein Y⁴ comprises a tetrazine and Y³ comprises a cyclooctene or a cyclopropene.
122. A method selected from:
   profiling a cell;
   examining a cell using a cytometry technique;
   diagnosing a disease or condition of a subject by examining pathology of a cell obtained from the subject;
   monitoring progression of disease or condition of a subject by examining pathology of a cell obtained from the subject; and
   detecting a disease biomarker in a cell;
      the method comprising:
         (i) obtaining a cell from the subject; and
         (ii) examining the cell according to the method of any one of paragraphs 103-121.
123. The method of paragraph 122, wherein the cell is obtained from the subject using image-guided biopsy, fine needle aspiration (FNA), surgical tissue harvesting, punch biopsy, liquid biopsy, brushing, swab, touch-prep, fluid aspiration or blood analysis.
124. The method of paragraph 122 or 123, wherein the cytometry technique is selected from image cytometry, holographic cytometry, Fourier ptychography cytometry, and fluorescence cytometry.
125. The method of any one of paragraphs 122-124, wherein the cell is selected from a cancer cell, an immune system cell, and a host cell.
126. The method of any one of paragraphs 122-124, wherein the disease or condition is cancer.
127. The method of paragraph 126, wherein the cancer is selected from lymphoma, breast cancer, skin cancer, lymphoma nodes, head and neck cancer, and oral cancer.
128. A method of preparing an activated ester of a compound comprising a carboxylic acid group, the method comprising
   i) reacting the compound comprising a carboxylic acid group with an excess amount of an activating reagent to obtain a reaction mixture comprising the activated ester; and
   ii) contacting the reaction mixture with a compound of Formula (D):

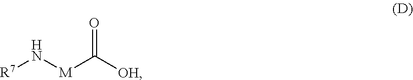

or a pharmaceutically acceptable salt thereof, wherein:
R⁷ is C₁₋₃ alkyl; and
M is C₂₋₆ alkylene;
wherein the contacting of the reaction mixture obtained in step i) with the compound of Formula (D), or a pharmaceutically acceptable salt thereof, deactivates the excess of the activating reagent in the reaction mixture.
129. The method of paragraph 128, wherein the activated ester is selected from N-hydroxysuccinimide (NHS) ester, nitrophenol ester, pentafluorophenol ester, and hydroxybenzotriazole ester.
130. The method of paragraph 128 or 129, wherein the compound comprising a carboxylic acid group is the compound of any one of paragraphs 1-65, or a pharmaceutically acceptable salt thereof, in which Y² is C(═O)OH.
131. The method of any one of paragraphs 128-130, wherein the activating reagent is selected from BOP, PyBOP, PyAOP, PyBrOP, BOP-Cl, HATU, HBTU, HCTU, TATU, TBTU, TDBTU, TSTU, TNTU, TPTU, DEPBT, and CDI, or a salt thereof.
132. The method of paragraph 131, wherein the activating reagent is TSTU:

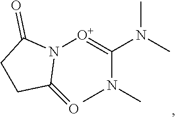

or a salt thereof.
133. The method of any one of paragraphs 128-132, wherein the compound of Formula (D) is a compound ENBA of formula:

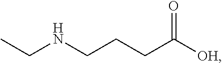

or a pharmaceutically acceptable salt thereof.
134. The method of any one of paragraphs 128-133, wherein the compound of Formula (D), or a pharmaceutically acceptable salt thereof, deactivates the excess of the activating agent by chemically reacting with the activating reagent.
135. The method of paragraph 134, wherein the chemical reaction between the compound of Formula (D) and the activating reagent produces a compound of Formula (E):

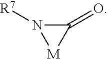

136. The method of paragraph 135, wherein the compound of formula (E) is:

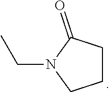

137. The method of paragraph 128, wherein:
   the activated ester is selected from N-hydroxysuccinimide (NHS) ester, nitrophenol ester, pentafluorophenol ester, and hydroxybenzotriazole ester;
   the activating reagent is selected from BOP, PyBOP, PyAOP, PyBrOP, BOP-Cl, HATU, HBTU, HCTU, TATU, TBTU, TDBTU, TSTU, TNTU, TPTU, DEPBT, and CDI, or a salt thereof, and
   the compound of Formula (D), or a pharmaceutically acceptable salt thereof, deactivates the excess of the activating reagent by chemically reacting with the activating reagent and forming a compound of Formula (E):

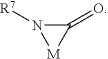

138. The method of paragraph 128, wherein:
   the activated ester is selected from N-hydroxysuccinimide (NHS) ester;
   the activating reagent is TSTU:

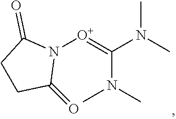

or a salt thereof;
the compound of Formula (D) is a compound ENBA of formula:

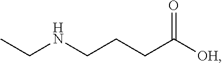

or a pharmaceutically acceptable salt thereof; and
the compound of Formula (D), or a pharmaceutically acceptable salt thereof, deactivates the excess of the activating agent by chemically reacting with the activating reagent and forming a compound of formula:

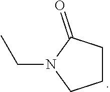

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A compound of Formula (A): 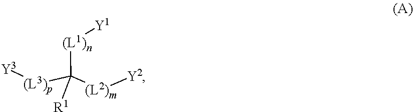
or a pharmaceutically acceptable salt thereof, wherein:
R¹ is selected from H, halo, C₁₋₆ alkyl, C₁₋₆ haloalkyl, C₁₋₆ alkoxy, and C₁₋₆ haloalkoxy;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
n is an integer from 1 to 10;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
m is an integer from 1 to 10;
each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ, —(CH₂CH(CH₃)O)ₓ, and a moiety formed by a click reaction, wherein said C₁₋₆ alkylene, C₃₋₇ cycloalkylene, C₆₋₁₀ arylene, —(OCH₂CH₂)ₓ, —(CH₂CH₂O)ₓ, —(OCH(CH₃)CH₂)ₓ, and —(CH₂CH(CH₃)O)ₓ— are each optionally substituted with 1, 2, 3, 4, or 5 substituents independently selected from OH, NH₂, C₁₋₆ alkylamino, di(C₁₋₆-alkyl)amino, C₁₋₆ haloalkyl, C₁₋₆ alkoxy, C₁₋₆ haloalkoxy, and (L⁴)ₒ-Y³;
each L⁴ is independently selected from N(R^{N1}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
p is an integer from 1 to 20;
is an integer from 1 to 10;
each x is independently an integer from 1 to 2,000;
each R^{N} is independently selected from H, C₁₋₃ alkyl, C₁₋₃ haloalkyl, and (L⁴)ₒ-Y³;
each R^{N1} is independently selected from H, C₁₋₃ alkyl, and C₁₋₃ haloalkyl;
Y¹ is selected from NR^{c1}R^{1A}, OR², and C(═O)R³;
R^{1A} selected from H, an amine protecting group, and a residue of a fluorophore;
R² is selected from H, an alcohol protecting group, and a residue of a fluorophore;
R³ is selected from OR^{a1} and a residue of a fluorophore;
Y² is selected from C(═O)OR^{a1}, NR^{c1}R⁴, OR⁵; and a group reactive with a side chain of an amino acid of a protein;
R^{a1} is selected from H and a carboxylic acid protecting group;
R^{c1} is selected from H, C₁₋₃ alkyl, and C₁₋₃ haloalkyl;
R⁴ is selected from H and an amine protecting group;
R⁵ is selected from H and an alcohol protecting group; and
Y³ is a chemical group that is reactive in a biorthogonal chemical reaction.

2. The compound of claim 1, wherein Y³ comprises a chemical group selected from an azide (—N₃), an aliphatic alkyne (—C≡CH), a cyclooctyne, a cyclooctene, a cyclohexene, a nitrone, an isocyanide, a cyclopropene, a norborene, a diphenylphosphine, nitrile imine, a tetrazole, a nitrile oxide, and a tetrazine.

3. The compound of claim 1, wherein the compound of Formula (A) has Formula (I): 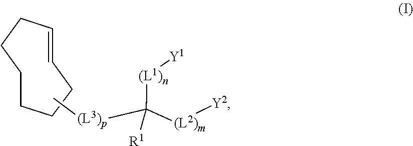
or a pharmaceutically acceptable salt thereof, wherein:
R¹ is selected from H, halo, C₁₋₆ alkyl, C₁₋₆ haloalkyl, C₁₋₆ alkoxy, and C₁₋₆ haloalkoxy;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
n is an integer from 1 to 10;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
m is an integer from 1 to 10;
each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
p is an integer from 1 to 10;
each x is independently an integer from 1 to 2,000;
each R^{N} is independently selected from H, C₁₋₃ alkyl, and C₁₋₃ haloalkyl;
Y¹ is selected from NR^{c1}R^{1A}, OR², and C(═O)R³;
R^{1A} selected from H, an amine protecting group, and a residue of a fluorophore;
R² is selected from H, an alcohol protecting group, and a residue of a fluorophore;
R³ is selected from OR^{a1} and a residue of a fluorophore;
Y² is selected from C(═O)OR^{a1}, NR^{c1}R⁴, OR⁵; and a group reactive with a side chain of an amino acid of a protein;
R^{a1} is selected from H and a carboxylic acid protecting group;
R^{c1} is selected from H, C₁₋₃ alkyl, and C₁₋₃ haloalkyl;
R⁴ is selected from H and an amine protecting group; and
R⁵ is selected from H an alcohol protecting group.

4. The compound of any one of claims 1-3, wherein:
R¹ is H;
n is an integer from 1 to 5, and each L¹ is selected from NH, O, C(═O), C₁₋₆ alkylene, and C₆₋₁₀ arylene;
m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—;
x is an integer from 2 to 10;
p is an integer from 1 to 15;
each L³ is independently selected from N(R^{N}), O, C(═O), C₁₋₆ alkylene, C₆₋₁₀ arylene, C₃₋₇ cycloalkylene, and a moiety formed by a click reaction, wherein said C₁₋₆ alkylene, C₃₋₇ cycloalkylene, and C₆₋₁₀ arylene are each optionally substituted with 1, 2, 3, or 4 substituents independently selected from OH and (L⁴)ₒ-Y³;
each R^{N} is independently selected from H and (L⁴)ₒ-Y³;
is an integer from 1 to 5; and
each L⁴ is independently selected from NH, O, C(═O), and C₁₋₆ alkylene.

5. The compound of claim 4, wherein:
R¹ is H;
n is an integer from 1 to 5, and each L¹ is selected from NH, O, C(═O), C₁₋₆ alkylene, and C₆₋₁₀ arylene;
m is an integer from 1 to 5, and each L² is independently selected from NH, O, C(═O), C₁₋₆ alkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—;
x is an integer from 2 to 10; and
p is an integer from 1 to 7, and each L³ is independently selected from NH, O, C(═O), C₁₋₆ alkylene, C₆₋₁₀ arylene, and —(OCH₂CH₂)ₓ—.

6. The compound of claim 4, wherein:
R¹ is H;
n is 1 and L¹ is C₁₋₆ alkylene;
m is 4, and each L² is independently selected from NH, C(═O), C₁₋₆ alkylene, and —(OCH₂CH₂)ₓ—;
p is 3, and each L³ is independently selected from NH, O, and C(═O); and
x is an integer from 2 to 10.

7. The compound of claim 1, wherein the compound of Formula (A) is selected from any one of the following compounds: 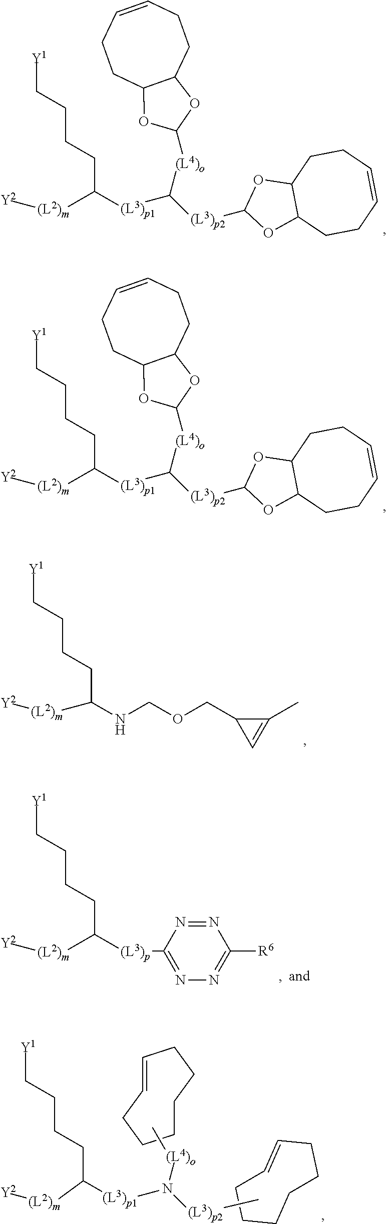
wherein:
the sum of p1 and p2 is less than p by at least 1, and
R⁶ is selected from H and C₁₋₆ alkyl.

8. The compound of claim 3, wherein the compound of Formula (I) is selected from any one of the following compounds: 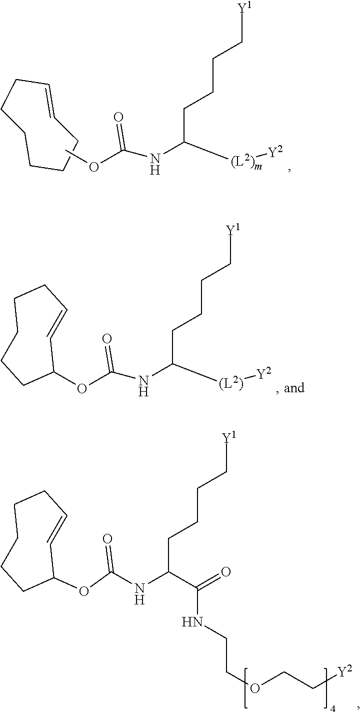
or a pharmaceutically acceptable salt thereof.

9. The compound of claim 1, wherein the compound of Formula (A) is selected from any one of the compounds depicted in FIGS. 1A, 15, 16A, 16B, 22A, 22B, 23-33, 35, and 36, and described in the examples 1, 3, 9, 10, 11, 12, and 13, or a pharmaceutically acceptable salt thereof.

10. The compound of claim 3, wherein the compound of Formula (I) is selected from any one of the compounds depicted in FIGS. 16A, 16B, 22A, and 22B, or a pharmaceutically acceptable salt thereof.

11. A protein conjugate of Formula (B): 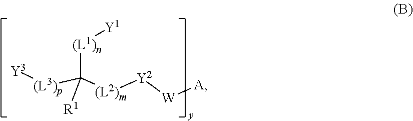
or a pharmaceutically acceptable salt thereof, wherein:
A is a residue of a protein;
y is an integer from 1 to 10;
R¹ is selected from H, halo, C₁₋₆ alkyl, C₁₋₆ haloalkyl, C₁₋₆ alkoxy, and C₁₋₆ haloalkoxy;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
n is an integer from 1 to 10;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
m is an integer from 1 to 10;
each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ, —(CH₂CH(CH₃)O)ₓ, and a moiety formed by a click reaction, wherein said C₁₋₆ alkylene, C₃₋₇ cycloalkylene, C₆₋₁₀ arylene, —(OCH₂CH₂)ₓ, —(CH₂CH₂O)ₓ, —(OCH(CH₃)CH₂)ₓ, and —(CH₂CH(CH₃)O)ₓ— are each optionally substituted with 1, 2, 3, 4, or 5 substituents independently selected from OH, NH₂, C₁₋₆ alkylamino, di(C₁₋₆-alkyl)amino, C₁₋₆ haloalkyl, C₁₋₆ alkoxy, C₁₋₆ haloalkoxy, and (L⁴)ₒ-Y³;
each L⁴ is independently selected from N(R^{N1}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
p is an integer from 1 to 20;
is an integer from 1 to 10;
each x is independently an integer from 1 to 2,000;
each R^{N} is independently selected from H, C₁₋₃ alkyl, C₁₋₃ haloalkyl, and (L⁴)ₒ-Y³;
each R^{N1} is independently selected from H, C₁₋₃ alkyl, and C₁₋₃ haloalkyl;
Y¹ is selected from NR^{c1}R^{1A}, OR², and C(═O)R³;
R^{1A}, R², and R³ are each independently a residue of a fluorophore;
R^{c1} is selected from H, C₁₋₃ alkyl, and C₁₋₃ haloalkyl;
each W is selected from:
(i) O of a side chain of serine, threonine, or tyrosine of the protein A;
(ii) S of a side chain of cysteine of the protein A;
(iii) NH of a side chain of lysine of the protein A; and
(iv) C(═O) of a side chain of aspartic acid or glutamic acid of the protein A;
Y² is a residue of a group which, prior to conjugation with the protein A, was a group reactive with a side chain of an amino acid of the protein A; and
Y³ is a chemical group that is reactive in a biorthogonal chemical reaction.

12. The protein conjugate of claim 11, wherein Y³ comprises a chemical group selected from an azide (—N₃), an aliphatic alkyne (—C≡CH), a cyclooctyne, a cyclooctene, a cyclohexene, a nitrone, an isocyanide, a cyclopropene, a norborene, a diphenylphosphine, nitrile imine, a tetrazole, a nitrile oxide, and a tetrazine.

13. The protein conjugate of claim 11, wherein the compound of Formula (B) has Formula (II): 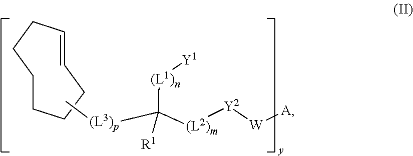
or a pharmaceutically acceptable salt thereof, wherein:
A is a residue of a protein;
y is an integer from 1 to 10;
R¹ is selected from H, halo, C₁₋₆ alkyl, C₁₋₆ haloalkyl, C₁₋₆ alkoxy, and C₁₋₆ haloalkoxy;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
n is an integer from 1 to 10;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
m is an integer from 1 to 10;
each L³ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ, —(OCH(CH₃)CH₂)ₓ—, and —(CH₂CH(CH₃)O)ₓ—,
p is an integer from 1 to 10;
each x is independently an integer from 1 to 2,000;
each R^{N} is independently selected from H, C₁₋₃ alkyl, and C₁₋₃ haloalkyl;
Y¹ is selected from NR^{c1}R^{1A}, OR², and C(═O)R³;
R^{1A}, R², and R³ are each independently a residue of a fluorophore;
R^{c1} is selected from H, C₁₋₃ alkyl, and C₁₋₃ haloalkyl;
each W is selected from:
(i) O of a side chain of serine, threonine, or tyrosine of the protein A;
(ii) S of a side chain of cysteine of the protein A;
(iii) NH of a side chain of lysine of the protein A; and
(iv) C(═O) of a side chain of aspartic acid or glutamic acid of the protein A;
Y² is a residue of a group which, prior to conjugation with the protein A, was a group reactive with a side chain of an amino acid of the protein A.

14. The protein conjugate of claim 11, wherein the protein conjugate of Formula (B) is selected from any one of the following formulae: 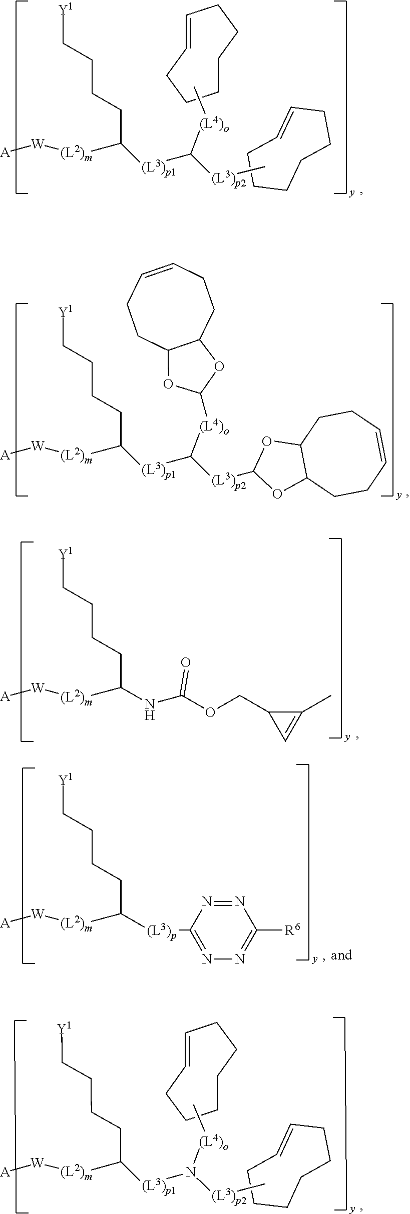
or a pharmaceutically acceptable salt thereof,
wherein:
the sum of p1 and p2 is less than p by at least 1, and
R⁶ is selected from H and C₁₋₆ alkyl.

15. The protein conjugate of claim 13, wherein the protein conjugate of Formula (II) is selected from any one of the following formulae: 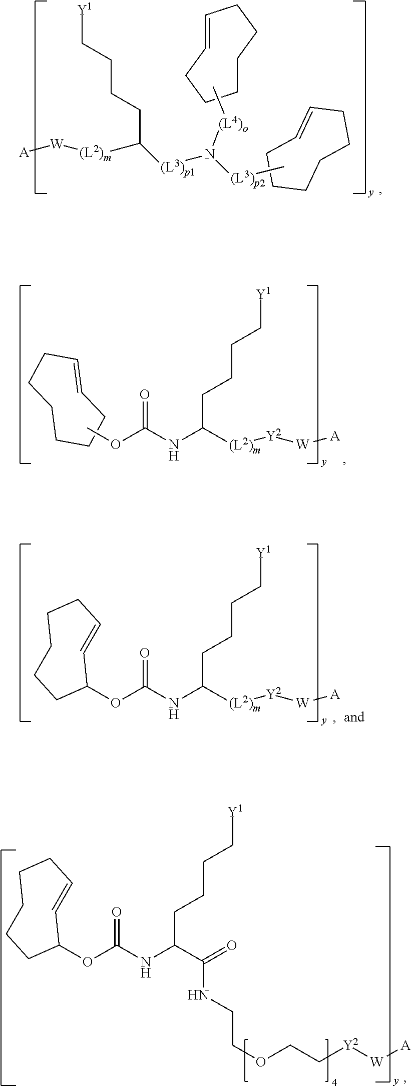
or a pharmaceutically acceptable salt thereof,
wherein:
the sum of p1 and p2 is less than p by at least 1, and

16. A method of examining a cell or a component of a cell, the method comprising:
(i) contacting the cell with a protein conjugate of any one of claims 11-15 comprising the residue of the fluorophore, or a pharmaceutically acceptable salt thereof,
(ii) imaging the cell with an imaging technique; and
(iii) after (ii), contacting the cell with a compound of Formula (C):
Y⁴-(L⁴)ₐ-Q (C),
or a pharmaceutically acceptable salt thereof, wherein:
each L⁴ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)_{y}—, —(CH₂CH₂O)_{y}—, —(OCH(CH₃)CH₂)_{y}—, and —(CH₂CH(CH₃)O)_{y}—,
a is an integer from 1 to 10;
each R^{N} is selected from H and C₁₋₃ alkyl;
each y is an integer from 1 to 2,000;
Q is a residue of a quencher; and
Y⁴ is a chemical group that is reactive in a biorthogonal chemical reaction,
wherein the contacting of step (iii) results in decrease of the fluorescence of the fluorophore in the conjugate of any one of claims 11-15, or a pharmaceutically acceptable salt thereof.

17. The method of claim 16, wherein Y⁴ comprises a chemical group selected from an azide (—N₃), an aliphatic alkyne (—C≡CH), a cyclooctyne, a cyclooctene, a cyclohexene, a nitrone, an isocyanide, a cyclopropene, a norborene, a diphenylphosphine, nitrile imine, a tetrazole, a nitrile oxide, and a tetrazine.

18. The method of claim 16, wherein Y⁴ in formula (C) is complementary to Y³ in the conjugate of any one of claims 11-15, or a pharmaceutically acceptable salt thereof.

19. The method of claim 16, comprising:
(i) contacting the cell with a conjugate of any one of claims 11-15 comprising the residue of the fluorophore, or a pharmaceutically acceptable salt thereof,
(ii) imaging the cell with an imaging technique; and
(iii) after (ii), contacting the cell with a compound of Formula (III): 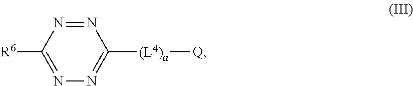
or a pharmaceutically acceptable salt thereof, wherein:
R⁶ is selected from H, C₁₋₆ alkyl, and C₁₋₆ haloalkyl;
each L⁴ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)_{y}—, —(CH₂CH₂O)_{y}—, —(OCH(CH₃)CH₂)_{y}—, and —(CH₂CH(CH₃)O)_{y}—,
a is an integer from 1 to 10;
each R^{N} is selected from H and C₁₋₃ alkyl;
each y is an integer from 1 to 2,000; and
Q is a residue of a quencher;
wherein the contacting of step (iii) results in decrease of the fluorescence of the fluorophore in the conjugate of any one of claims 11-15, or a pharmaceutically acceptable salt thereof.

20. A method selected from:
profiling a cell;
examining a cell using a cytometry technique;
diagnosing a disease or condition of a subject by examining pathology of a cell obtained from the subject;
monitoring progression of disease or condition of a subject by examining pathology of a cell obtained from the subject; and
detecting a disease biomarker in a cell;
the method comprising:
(i) obtaining a cell from the subject; and
(ii) examining the cell according to the method of any one of claims 16-19.

21. The method of claim 20, wherein the cell is obtained from the subject using image-guided biopsy, fine needle aspiration (FNA), surgical tissue harvesting, punch biopsy, liquid biopsy, brushing, swab, touch-prep, fluid aspiration or blood analysis.

22. The method of claim 20, wherein the cell is selected from a cancer cell, an immune system cell, and a host cell.

23. The method of claim 20, wherein the protein in the conjugate of any one of claims 11-15 is selected from an antibody, an antibody fragment, an engineered antibody, a peptide, and an aptamer.

24. The method of claim 23, wherein the antibody is specific to an antigen which is a biomarker of a disease or condition.

25. The method of claim 24, wherein the disease or condition is cancer.

26. The method of claim 25, wherein the cancer is selected from lymphoma, breast cancer, skin cancer, lymphoma nodes, head and neck cancer, and oral cancer.

27. The method of claim 24, wherein:
the biomarker of the disease or condition is PD-L¹; and
the disease or condition is head and neck squamous cell carcinoma (HNSCC).

28. A method of preparing an activated ester of a compound comprising a carboxylic acid group, the method comprising
i) reacting the compound comprising a carboxylic acid group with an excess amount of an activating reagent to obtain a reaction mixture comprising the activated ester; and
ii) contacting the reaction mixture with a compound of Formula (D): 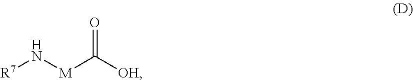
or a pharmaceutically acceptable salt thereof, wherein:
R⁷ is C₁₋₃ alkyl; and
M is C₂₋₆ alkylene;
wherein the contacting of the reaction mixture obtained in step i) with the compound of Formula (D), or a pharmaceutically acceptable salt thereof, deactivates the excess of the activating reagent in the reaction mixture.

29. The method of claim 28, wherein
the activated ester is selected from N-hydroxysuccinimide (NHS) ester, nitrophenol ester, pentafluorophenol ester, and hydroxybenzotriazole ester;
the activating reagent is selected from BOP, PyBOP, PyAOP, PyBrOP, BOP-Cl, HATU, HBTU, HCTU, TATU, TBTU, TDBTU, TSTU, TNTU, TPTU, DEPBT, and CDI, or a salt thereof, and
the compound of Formula (D), or a pharmaceutically acceptable salt thereof, deactivates the excess of the activating reagent by chemically reacting with the activating reagent and forming a compound of Formula (E):

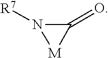

30. The method of claim 28, wherein:
the activated ester is selected from N-hydroxysuccinimide (NHS) ester;
the activating reagent is TSTU: 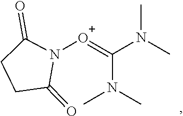
or a salt thereof;
the compound of Formula (D) is a compound ENBA of formula: 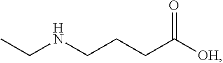
or a pharmaceutically acceptable salt thereof; and
the compound of Formula (D), or a pharmaceutically acceptable salt thereof, deactivates the excess of the activating agent by chemically reacting with the activating reagent and forming a compound of formula: [chemical structure]
